(19) United States
(12) Patent Application Publication
(10) Pub. No.: US 2026/0115152 A1
(43) Pub. Date: Apr. 30, 2026

(54) AGRIMOL B DERIVATIVES AND METHODS OF MAKING AND USE THEREOF

(71) Applicant: Laboratory for Synthetic Chemistry and Chemical Biology Limited, Hong Kong (CN)

(72) Inventors: Chi-Ming Che, Hong Kong (CN); Pui Kin Tony Lo, Hong Kong (CN)

(21) Appl. No.: 19/369,624

(22) Filed: Oct. 27, 2025

Related U.S. Application Data
(60) Provisional application No. 63/713,332, filed on Oct. 29, 2024.

(51) Int. Cl. A61K 31/121; A61P 35/00; C07C 315/02; C07C 317/14

(52) U.S. Cl. CPC A61K 31/121; A61P 35/00; C07C 315/02; C07C 317/14; C07C 2601/14; C07C 2601/16

(57) ABSTRACT

Agrimol B (“AGB”) derivatives that contain a sulfone moiety and show improved stability compared to its parent AGB are disclosed. In some forms, the AGB derivatives can possess anticancer properties that are comparable to its parent AGB. Methods for synthesizing the AGB derivatives using a sulfone intermediate or ketone/sulfone intermediate are disclosed. Methods of using the AGB derivatives for treating a cancer, such as large cell lung cancer, in a subject are also disclosed.

### CROSS REFERENCE TO RELATED APPLICATION

This application claims priority to U.S. Provisional Patent Application No. 63/713,332 filed Oct. 29, 2024, the disclosure of which is incorporated herein by reference.

### FIELD OF THE INVENTION

This invention is generally in the field of Agrimol B derivatives and methods of synthesis and uses thereof.

### BACKGROUND OF THE INVENTION

Agrimol B (AGB) is a natural compound originally found in a plant called Agrimonia pilosa, and it has shown promise in fighting lung cancer. However, the ketone group of AGB can easily react and break down during manufacturing and inside the body, making it less effective as a therapeutic.

There remains a need to develop AGB derivative that have improved stability and process anticancer properties, and their synthesis thereof.

Therefore, it is the object of the present invention to provide AGB derivatives that have improved stability (e.g., chemical stability and/or metabolic stability).

It is a further object of the present invention to provide AGB derivatives that possess anticancer properties.

It is a further object of the present invention to provide methods of synthesizing the AGB derivatives.

It is a further object of the present invention to provide methods of using the AGB derivatives as anticancer agents.

### SUMMARY OF THE INVENTION

AGB derivatives (also referred to herein as “compounds”) have been developed. The AGB derivatives disclosed herein contain a sulfone moiety, and show improved stability (e.g., chemical stability and/or metabolic stability) compared to its parent AGB. For example, compared to AGB, the AGB derivatives containing the sulfone moiety are more soluble in water, less prone to metabolic degradation with the body, and/or more likely to remain unchanged through prolonged organic reactions. The advantages of the AGB derivatives allow them to be synthesized more easily and stay active in the body longer that can lead to improved ADME (absorption, distribution, metabolism, and excretion) profile of these compounds.

Without being bound to any theories, it is believed that the improved chemical/metabolic stability and solubility of the AGB derivatives is attributed to the sulfone moiety that is inert to most traditional reactions involving ketones, such as nucleophilic attacks and Grignard additions, and is more polar that reduces lipophilicity and increases the water solubility of the compound.

In some forms, the AGB derivatives possess anticancer properties that are comparable to its parent AGB, and are useful in anticancer treatments. For example, the AGB derivatives show cytotoxicity against cancer cells (e.g., as indicated by its IC₅₀ values), such as large cell lung cancer cells (e.g., NCI-H460 cells), similar to that of AGB (e.g., concentration in μM on the same order of magnitude).

In some forms, the AGB derivatives can have the structure of Formula I:

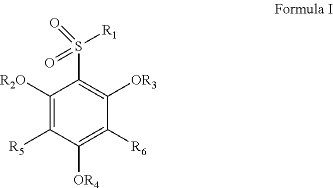

wherein: (i) R₁ can be an alkyl or

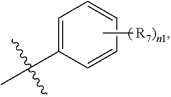

n1 can be an integer from 1 to 5, and R₇ can be hydrogen, an alkyl, —O-alkyl, hydroxyl, or —NR′₁R′₂, wherein R′₁ and R′₂ can be independently hydrogen or an alkyl; (ii) R₂, R₃, and R₄ can be independently hydrogen or an alkyl; and (iii) R₅ and R₆ can be independently an alkyl, —C(O)—R₁₂, or

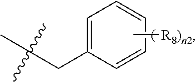

n2 can be an integer from 1 to 5, R₈ can be hydrogen, hydroxyl, an alkyl, —O-alkyl, or —C(O)—R₉, R₉ and R₁₂ can be independently hydrogen, hydroxyl, an alkyl, or —O-alkyl. In some forms, at least one of R₅ and R₆ can be

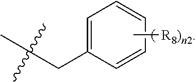

In some forms, the

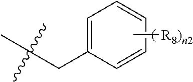

can be

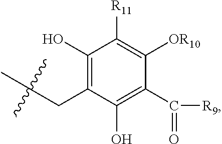

R₁₀ can be hydrogen or an alkyl, and R₉ and R₁₁ can be independently hydrogen, hydroxyl, or an alkyl. In some forms, R₉-R₁₁ can be independently an alkyl. In some forms, the

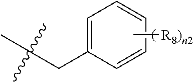

can be

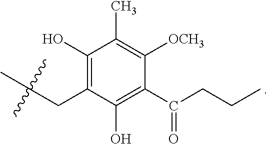 [SMILES: *Cc1c(O)c(C)c(OC)c(C(=O)CCC)c1O]

In some forms, the

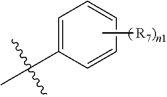

can be

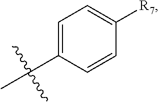

and R₇ can be hydrogen, an alkyl, —O-alkyl, hydroxyl, or —NR′₁R′₂, R′₁ and R′₂ can be independently hydrogen or an alkyl. In some forms, R₇ can be hydrogen, an alkyl, or —O-alkyl.

In some forms, R₂-R₄ can be hydrogen.

In some forms, R₁₂ can be an alkyl.

In some forms, the AGB derivative can have any one of the following structures:

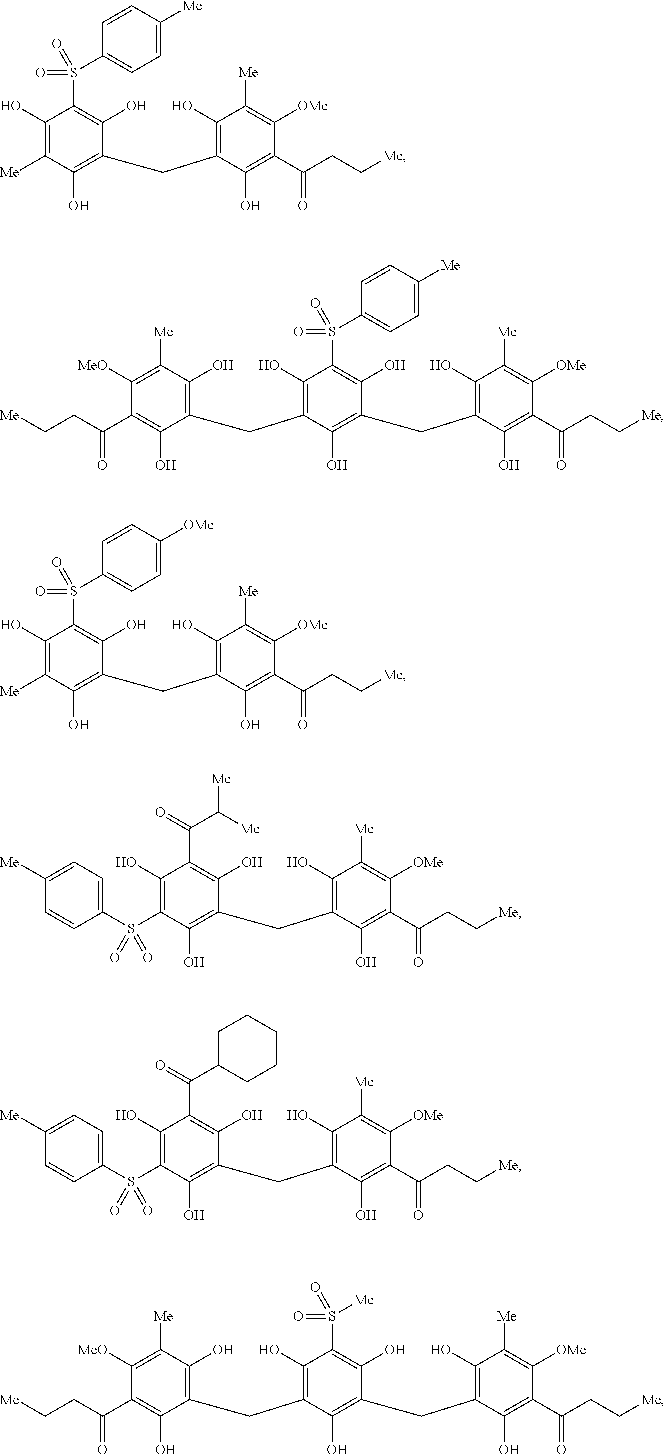

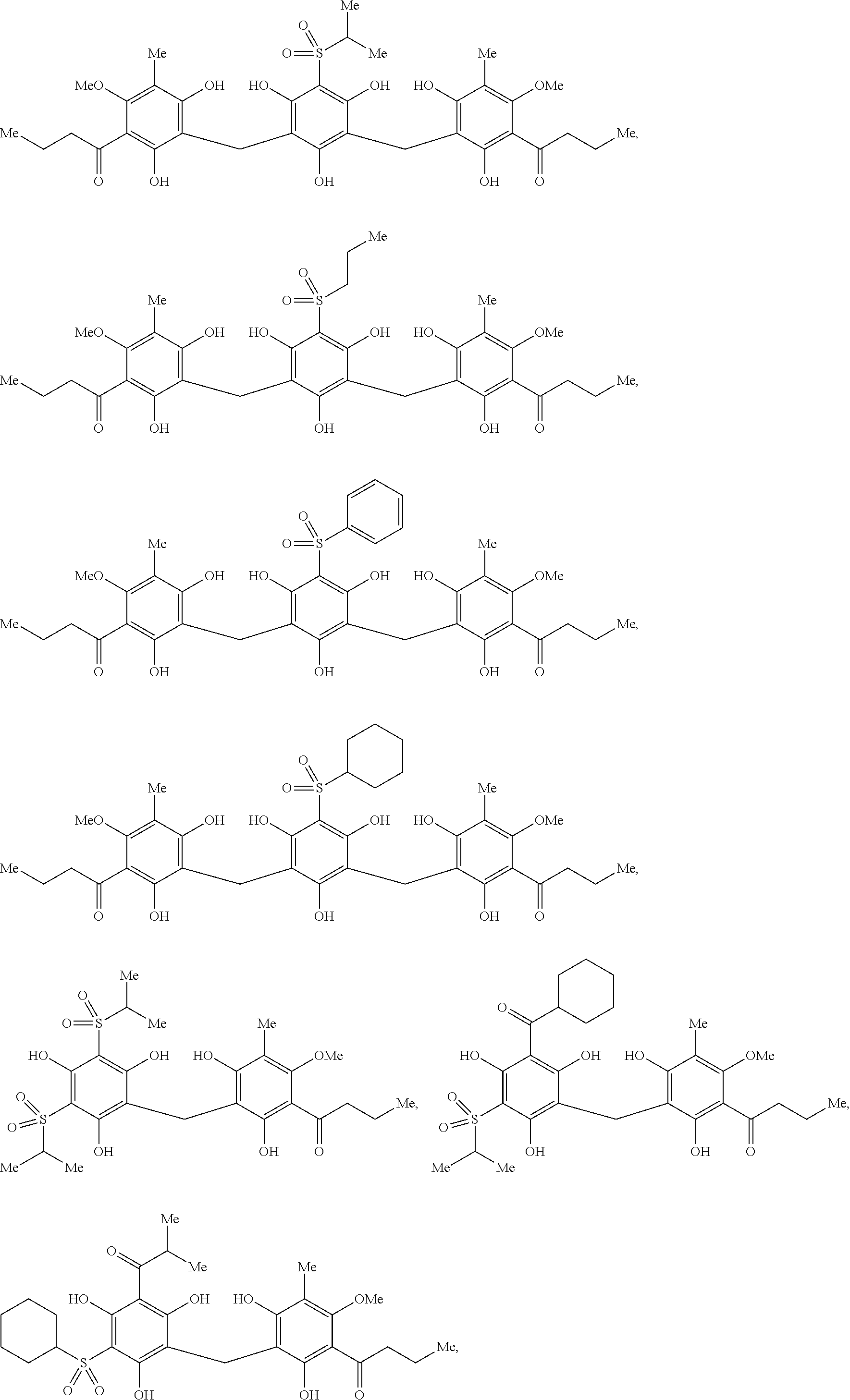

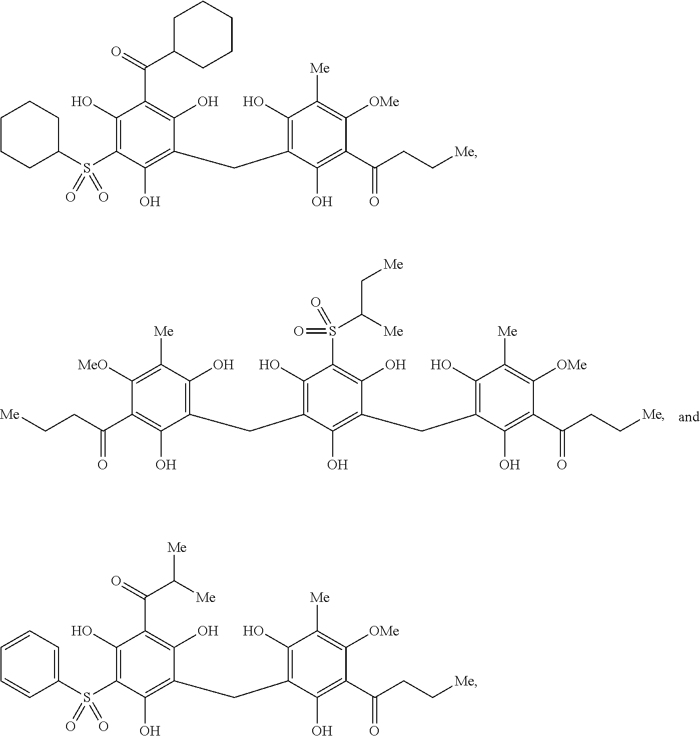 [SMILES: CCCC(=O)c1c(O)c(Cc2c(O)c(C(=O)C3CCCCC3)c(O)c(S(=O)(=O)C3CCCCC3)c2O)c(O)c(C)c1OC ; CCCC(=O)c1c(O)c(Cc2c(O)c(Cc3c(O)c(C)c(OC)c(C(=O)CCC)c3O)c(O)c(S(=O)(=O)C(C)CC)c2O)c(O)c(C)c1OC ; CCCC(=O)c1c(O)c(Cc2c(O)c(C(=O)C(C)C)c(O)c(S(=O)(=O)c3ccccc3)c2O)c(O)c(C)c1OC]

Methods for synthesizing the AGB derivatives are disclosed. Generally, the synthesis includes (1) converting a starting reagent to a sulfone intermediate or a ketone/sulfone intermediate; and (2) converting the sulfone intermediate or ketone/sulfone intermediate to the compound. The disclosed methods employ easy-to-follow synthetic paths using commercially available and easy-to-handle reagents.

In some forms, the sulfone intermediate is an aryl sulfone intermediate, which may be prepared in a single step. For example, the sulfone intermediate produced in step (1) is an aryl sulfone intermediate that is prepared following a direct Friedel-Crafts sulfonylation reaction. This reaction can be applied to aryl with both electron-neutral and electron-donating groups and excludes an oxidation process that is required in previous methods (H. Burton and E. Hoggarth, Journal of the Chemical Society, 1945, 468-470).

In some forms, the sulfone intermediate is an alkyl sulfone intermediate using a sulfur dioxide surrogate to introduce the sulfonyl (—SO₂—) group, producing lithium sulfinate (RSO₂Li) as a versatile intermediate, which then reacts with an alkyl electrophiles to produce the alkyl sulfone intermediate. In these forms, a deprotection step may be further performed with a suitable agent, such as BBr₃, to convert one or more —O— alkyl group to —OH.

Methods of using the AGB derivatives or a pharmaceutical composition thereof for treating cancer in a subject in need thereof are also disclosed.

### DETAILED DESCRIPTION OF THE INVENTION

### I. Definitions

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

“Substituted alkyl” refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF3, —CH₂—CF₃, —CCl₃); —CN; —NCOCOCH₂CH₂; —NCOCOCHCH; and —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom on the carbon backbone. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C═C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl.”

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. Asymmetric structures such as (AB)C≡C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl.”

The term “substituted alkynyl” refers to alkynyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkynyl,” as used herein, refers to straight or branched chain, or cyclic carbon-containing alkynyl radicals, or combinations thereof, containing at least one heteroatom. Suitable heteroatoms include, but are not limited to, 0, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

“Aryl,” as used herein, refers to C₄-C₂₆-membered aromatic rings or fused ring systems containing one aromatic ring and optionally one or more non-aromatic rings. Examples of aryl groups are benzene, tetralin, indane, etc.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocyclo” and “heterocyclyl” are used interchangeably, and refer to a cyclic radical attached via a ring carbon or nitrogen atom of a monocyclic ring or polycyclic ring system containing 3-30 ring atoms, 3-20 ring atoms, 3-10 ring atoms, or 5-6 ring atoms, where the polycyclic ring system contains one or more non-aromatic rings and optionally one or more aromatic rings, where at least one non-aromatic ring contains carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc., such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₃-C₂₆-membered aromatic rings or fused ring systems containing one aromatic ring and optionally one or more non-aromatic rings, in which one or more carbon atoms on the aromatic ring structure have been substituted with a heteroatom. Suitable heteroatoms include, but are not limited to, oxygen, sulfur, and nitrogen. Examples of heteroaryl groups pyrrole, furan, thiophene, imidazole, oxazole, thiazole, triazole, tetrazole, pyrazole, pyridine, pyrazine, pyridazine and pyrimidine, and the like. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

The term “polyaryl” refers to a fused ring system that includes two or more aromatic rings and optionally one or more non-aromatic rings. Examples of polyaryl groups are naphthalene, anthracene, phenanthrene, chrysene, pyrene, corannulene, coronene, etc. When a fused ring system containing two or more aromatic rings and optionally one or more non-aromatic rings, in which one or more carbon atoms on two or more aromatic ring structures have been substituted with a heteroatom, the fused ring system can be referred to as a “polyheteroaryl”. When a fused ring system containing two or more aromatic rings and optionally one or more non-aromatic rings, in which one or more carbon atoms in the fused ring system is substituted with a heteroatom it can be referred to as a “heteropolyaryl.”

The term “substituted polyaryl” refers to a polyaryl in which one or more of the aryls are substituted, with one or more substituents including, but not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof. When a polyheteroaryl is involved, the chemical moiety can be referred to as a “substituted polyheteroaryl.”

The term “cyclic ring” or “cyclic group” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, or a substituted or unsubstituted heterocyclyl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, and heterocyclyls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “alkoxyl” or “alkoxy,” “aroxy” or “aryloxy,” generally describe compounds represented by the formula —OR^{v}, wherein R includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a phosphonium, a phosphanyl, a phosphonyl, a sulfinyl, a silyl, a thiol, an amido, and an amino. Exemplary alkoxyl groups include methoxy, ethoxy, propyloxy, tert-butoxy and the like. A “lower alkoxy” group is an alkoxy group containing from one to six carbon atoms. An “ether” is two functional groups covalently linked by an oxygen as defined below. Accordingly, the substituent of an alkyl that renders that alkyl an ether is or resembles an alkoxyl, such as can be represented by one of —O-alkyl, —O-alkenyl, —O-alkynyl, —O— arakyl, —O-aryl, —O-heteroaryl, —O-polyaryl, —O-polyheteroaryl, —O-heterocyclyl, etc.

The term “substituted alkoxy” refers to an alkoxy group having one or more substituents replacing one or more hydrogen atoms on one or more carbons of the alkoxy backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, oxo, sulfate, sulfonate, sulfamoyl, sulfonamido, sulfonyl, heterocyclyl, alkylaryl, haloalkyl, —CN, aryl, heteroaryl, and combinations thereof.

The term “ether” as used herein is represented by the formula A²OA¹, where A² and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a phosphonyl, a sulfinyl, a silyl, a thiol, a substituted or unsubstituted carbonyl, an alkoxy, an amido, or an amino, described above.

The term “polyether” as used herein is represented by the formula:

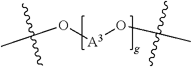

where A³ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a substituted or unsubstituted carbonyl, an alkoxy, an amido, or an amino, described above; g can be a positive integer from 1 to 30.

The term “phenoxy” is art recognized and refers to a compound of the formula —OR^{v} wherein R^{v} is C₆H₅ (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “amino” as used herein includes the group

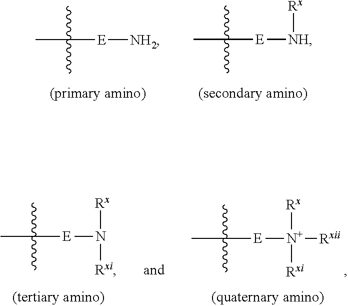

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The terms “amide” or “amido” are used interchangeably, refer to both “unsubstituted amido” and “substituted amido” and are represented by the general formula:

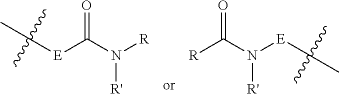

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted aryl, substituted or unsubstituted heteroaryl, a substituted or unsubstituted polyaryl, a substituted or unsubstituted polyheteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. In some forms, when E is oxygen, a carbamate is formed. It is understood by those of ordinary skill in the art, that the E groups listed above are divalent (e.g., methylene, ethane-1,2-diyl, ethene-1,2-diyl, 1,4-phenylene, cyclohexane-1,2-diyl).

“Carbonyl,” as used herein, is art-recognized and includes such moieties as can be represented by the general formula:

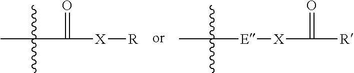

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″, or a pharmaceutical acceptable salt; E″ is absent, or E″ is a substituted or unsubstituted alkyl, a substituted or unsubstituted alkenyl, a substituted or unsubstituted alkynyl, a substituted or unsubstituted aralkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″; R″ represents a hydroxyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. It is understood by those of ordinary skill in the art, that the E″ groups listed above are divalent (e.g., methylene, ethane-1,2-diyl, ethene-1,2-diyl, 1,4-phenylene, cyclohexane-1,2-diyl). Where X is oxygen and R is defined as above, the moiety is also referred to as a carboxyl group. When X is oxygen and R is hydrogen, the formula represents a “carboxylic acid.” Where X is oxygen and R′ is hydrogen, the formula represents a “formate.” Where X is oxygen and R or R′ is not hydrogen, the formula represents an “ester.” In general, where the oxygen atom of the above formula is replaced by a sulfur atom, the formula represents a “thiocarbonyl” group. Where X is sulfur and R or R′ is not hydrogen, the formula represents a “thioester.” Where X is sulfur and R is hydrogen, the formula represents a “thiocarboxylic acid.” Where X is sulfur and R′ is hydrogen, the formula represents a “thioformate.” Where X is a bond and R is not hydrogen, the above formula represents a “ketone.” Where X is a bond and R is hydrogen, the above formula represents an “aldehyde.”

The term “phosphanyl” is represented by the formula

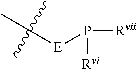

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi} and R^{vii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonium” is represented by the formula

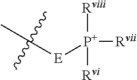

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi}, R^{vii}, and R^{viii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi}, R^{vii}, and R^{viii} taken together with the P⁺ atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonyl” is represented by the formula

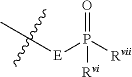

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, oxygen, alkoxy, aroxy, or substituted alkoxy or substituted aroxy, wherein, independently of E, R^{vi} and R^{vii} are independently a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphoryl” defines a phosphonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as 20 understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfinyl” is represented by the formula

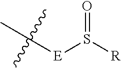

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a silyl, a thiol, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonyl” is represented by the formula

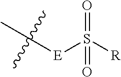

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, a substituted or unsubstituted polyaryl, a substituted or unsubstituted polyheteroaryl, or substituted or unsubstituted heteroaryl. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amino, substituted or unsubstituted cycloalkyl, substituted or unsubstituted heterocyclyl, substituted or unsubstituted aralkyl, substituted or unsubstituted alkylaryl, substituted or unsubstituted arylalkyl, substituted or unsubstituted aryl, substituted or unsubstituted heteroaryl, a substituted or unsubstituted polyaryl, a substituted or unsubstituted polyheteroaryl, —(CH₂)ₘ—R′″, R′″ represents a hydroxy group, substituted or unsubstituted carbonyl group, an aryl, a cycloalkyl ring, a cycloalkenyl ring, a heterocycle, an amido, an amino, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfamoyl” refers to a sulfonamide or sulfonamide represented by the formula

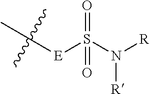

wherein E is absent, or E is substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted cycloalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “silyl group” as used herein is represented by the formula —SiRR′R,″ where R, R′, and R″ can be, independently, a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, a phosphonyl, a sulfinyl, a thiol, an amido, an amino, an alkoxy, or an oxo, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted, independently, with the substituents described above in the definition of “substituted.”

The numerical ranges disclose individually each possible number that such a range could reasonably encompass, as well as any sub-ranges and combinations of sub-ranges encompassed therein. For example, in a given range carbon range of C₃-C₉, the range also discloses C₃, C₄, C₅, C₆, C₇, C₈, and C₉, as well as any subrange between these numbers (for example, C₄-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted with, independently, with the substituents described above in the definition of “substituted.”

“oxo” refers to ═O.

The compounds and substituents can be substituted, independently, with the substituents described above in the definition of “substituted.”

Numerical ranges such as ranges of C₁-C₃₀, C₄-C₃₀, C₃-C₃₀, C₁-C₂₀, C₄-C₂₀, C₃-C₂₀, C₁-C₁₀, C₄-C₁₀, C₃-C₁₀, C₁-C₆, C₄-C₆, C₃-C₆, C₁-C₄, C₃-C₄, C₁-C₉, C₁-C₈, C₁-C₇, C₁-C₅, C₁-C₃, C₁-C₂, C₃-C₉, C₃-C₉, C₃-C₈, C₃-C₇, C₃-C₅, C₃-C₄, C₄-C₂₅, C₄-C₂₀, C₄-C₁₈, C₄-C₁₆, C₄-C₁₅, C₄-C₁₄, C₄-C₁₃, C₄-C₁₂, C₄-C₉, C₄-C₈, C₄-C₇, C₄-C₅, etc. The ranges disclose individually each possible number that such a range could reasonably encompass, as well as any sub-ranges and combinations of sub-ranges encompassed therein. For example, in a given range carbon range of C₃-C₉, the range also discloses C₃, C₄, C₅, C₆, C₇, C₈, and C₉, as well as any subrange between these numbers (for example, C₄-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

### II. Compositions

AGB derivatives (also referred to herein as “compounds”) have been developed. The AGB derivatives disclosed herein contain a sulfone moiety, and show improved stability (e.g., chemical stability and/or metabolic stability) compared to its parent AGB. For example, compared to AGB, the AGB derivatives containing the sulfone moiety are more soluble in water, less prone to metabolic degradation with the body, and/or more likely to remain unchanged through prolonged organic reactions. The advantages of the AGB derivatives allow them to be synthesized more easily and stay active in the body longer that can lead to improved ADME (absorption, distribution, metabolism, and excretion) profile of these compounds.

Without being bound to any theories, it is believed that the improved chemical/metabolic stability and solubility of the AGB derivatives is attributed to the sulfone moiety that is inert to most traditional reactions involving ketones, such as nucleophilic attacks and Grignard additions, and is more polar that reduces lipophilicity and increases the water solubility of the compound.

In some forms, the AGB derivatives possess anticancer properties that are comparable to its parent AGB, and are useful in anticancer treatments. For example, the AGB derivatives show cytotoxicity against cancer cells (e.g., as indicated by its IC₅₀ values), such as large cell lung cancer cells (e.g., NCI-H460 cells), similar to that of AGB (e.g., concentration in μM on the same order of magnitude).

Pharmaceutical formulations containing the AGB derivatives are also disclosed.

### A. Agrimol B Derivatives

In some forms, the AGB derivatives thereof (also referred to herein as “compounds”) can have the structures of Formula I:

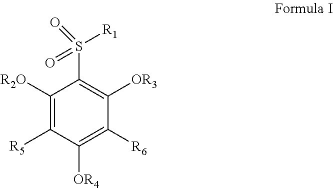

wherein: (i) R₁ can be an alkyl (e.g., methyl, ethyl, n-propyl, isopropyl, n-butyl, isobutyl, tert-butyl, cyclopentyl, cyclohexyl, etc., for example, methyl, n-propyl, isopropyl, isobutyl, or cyclohexyl) or

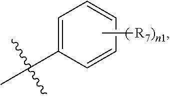

n1 can be an integer from 1 to 5, and R₇ can be hydrogen, an electron neutral group, or an electron donating group, such as an alkyl, —O-alkyl, hydroxyl, or —NR′₁R′₂, wherein R′₁ and R′₂ can be independently hydrogen or an alkyl; (ii) R₂, R₃, and R₄ can be independently hydrogen or an alkyl; and (iii) R₅ and R₆ can be independently an alkyl, —C(O)—R₁₂, or

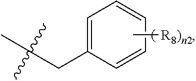

n2 can be an integer from 1 to 5, R₈ can be hydrogen, hydroxyl, an alkyl, —O-alkyl, or —C(O)—R₉, R₉ and R₁₂ can be independently hydrogen, hydroxyl, an alkyl, or —O-alkyl. In some preferred forms, at least one of R₅ and R₆ is

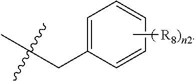

In some forms, the

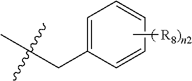

can be

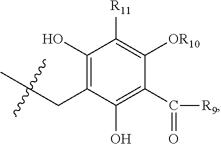

R₁₀ can be hydrogen or an alkyl, and R₉ and R₁ can be independently hydrogen, hydroxyl, or an alkyl. In some forms, R₉-R₁₁ can be independently an alkyl, such as methyl, ethyl, n-propyl, isopropyl, n-butyl, isobutyl, tert-butyl, cyclopentyl, cyclohexyl, etc. In some forms, the

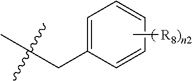

can be

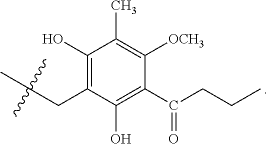 [SMILES: *Cc1c(O)c(C)c(OC)c(C(=O)CCC)c1O]

In some forms, the

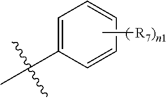

can be

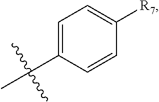

and R₇ can be hydrogen, an alkyl, —O-alkyl, hydroxyl, or —NR′₁R′₂, R′₁ and R′₂ can be independently hydrogen or an alkyl. In some forms, R₇ can be hydrogen, an alkyl, or —O-alkyl. In some forms, R₇ can be hydrogen, methyl, ethyl, n-propyl, isopropyl, n-butyl, isobutyl, tert-butyl, cyclopentyl, cyclohexyl, methoxy, or ethoxy, for example, hydrogen, methyl, cyclohexyl, or methoxy.

In some forms, R₂-R₄ can be hydrogen.

In some forms, R₁₂ can be an alkyl. In some forms, R₁₂ can be methyl, ethyl, n-propyl, isopropyl, n-butyl, isobutyl, tert-butyl, cyclopentyl, or cyclohexyl. In some forms, R₁₂ can be methyl, isopropyl, isobutyl, or cyclohexyl.

For any forms of the compounds disclosed herein, when the R group is or contains an alkyl, the alkyl can be a substituted alkyl or an unsubstituted alkyl. In some forms, the alkyl can be an unsubstituted alkyl. In some forms, the alkyl can be a substituted alkyl, where the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl (e.g. benzyl), a carbonyl (e.g. carboxyl), an alkoxy (e.g. methoxy, ethoxy, aryloxy, benzoether, etc.), a halide, a hydroxyl, or a haloalkyl, or a combination thereof. In some forms, the alkyl can be a substituted alkyl, where the substituents can be independently an unsubstituted alkyl, an unsubstituted alkenyl, an unsubstituted alkynyl, an unsubstituted heterocyclyl, an unsubstituted aryl, an unsubstituted heteroaryl, an unsubstituted polyaryl, an unsubstituted polyheteroaryl, an unsubstituted aralkyl (e.g. benzyl), an alkoxy (e.g. methoxy, ethoxy, aryloxy, benzoether, etc.), a carbonyl (e.g. carboxyl), a halide, a hydroxyl, or a haloalkyl, or a combination thereof. In some forms, the alkyl can be a substituted alkyl, where the substituents can be an unsubstituted alkyl (e.g. methyl, ethyl, propyl, butyl, pentyl, hexyl, etc.), an alkoxy (e.g. methoxy, ethoxy, etc.), a hydroxyl, or a combination thereof.

For any forms of the compounds disclosed herein, when the R group is or contains an alkyl, the alkyl can be a linear alkyl, a branched alkyl, or a cyclic alkyl (either monocyclic or polycyclic). The terms “cyclic alkyl” and “cycloalkyl” are used interchangably herein. Exemplary alkyl include a linear C₁-C₃₀ alkyl, a branched C₄-C₃₀ alkyl, a cyclic C₃-C₃₀ alkyl, a linear C₁-C₂₀ alkyl, a branched C₄-C₂₀ alkyl, a cyclic C₃-C₂₀ alkyl, a linear C₁-C₁₀ alkyl, a branched C₄-C₁₀ alkyl, a cyclic C₃-C₁₀ alkyl, a linear C₁-C₆ alkyl, a branched C₄-C₆ alkyl, a cyclic C₃-C₆ alkyl, a linear C₁-C₄ alkyl, cyclic C₃-C₄ alkyl, such as a linear C₁-C₁₀, C₁-C₉, C₁-C₈, C₁-C₇, C₁-C₆, C₁-C₅, C₁-C₄, C₁-C₃, or C₁-C₂ alkyl group, a branched C₃-C₉, C₃-C₉, C₃-C₈, C₃-C₇, C₃-C₆, C₃-C₅, or C₃-C₄ alkyl group, or a cyclic C₃-C₉, C₃-C₉, C₃-C₈, C₃-C₇, C₃-C₆, C₃-C₅, or C₃-C₄ alkyl group. The cyclic alkyl can be a monocyclic or polycyclic alkyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄-C₅ monocyclic or polycyclic alkyl group.

For example, for any forms of the compounds disclosed herein, when the R group is or contains an alkyl, the alkyl is an unsubstituted C₁-C₈ alkyl, an unsubstituted C₁-C₆ alkyl, an unsubstituted C₁-C₄ alkyl, an unsubstituted C₁-C₃ alkyl, an ethyl, or a methyl, for example, methyl, ethyl, n-propyl, isopropyl, n-butyl, isobutyl, tert-butyl, cyclohexyl, etc.

In some forms, the AGB derivative can have any one of the following structures:

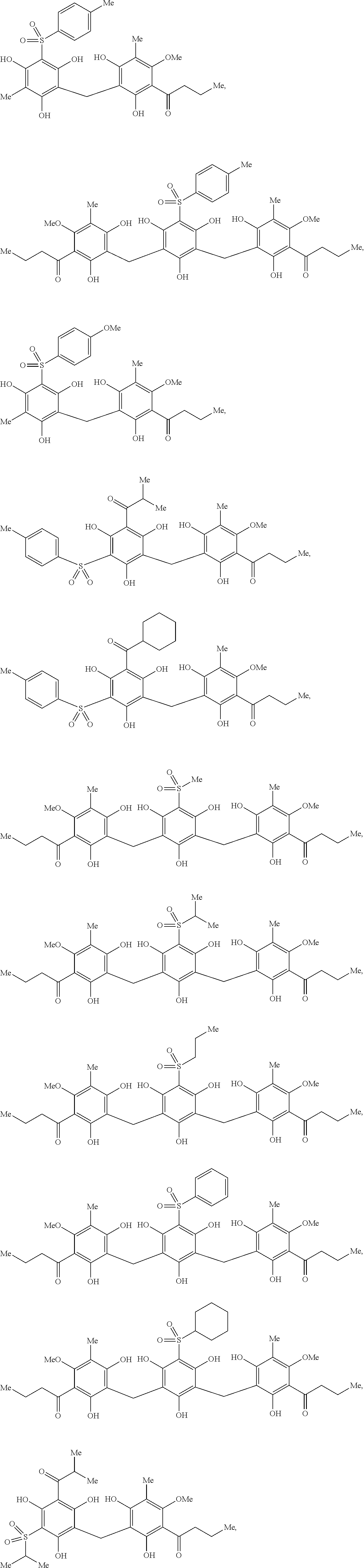

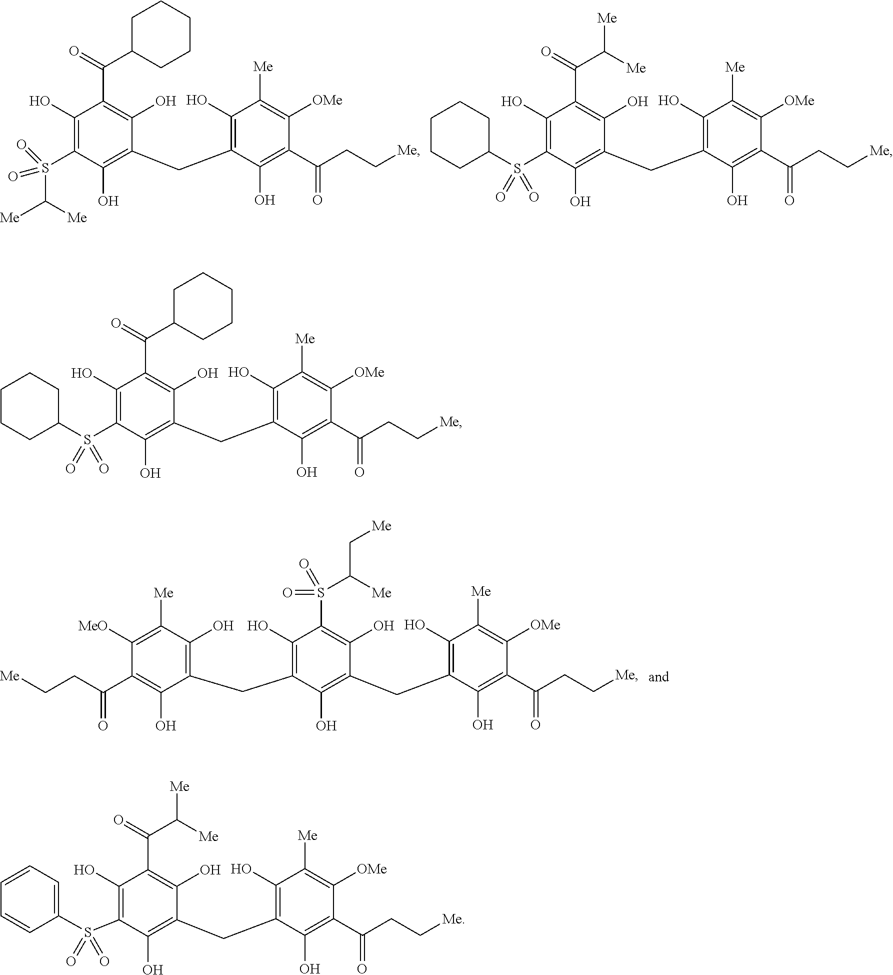

The compounds may be neutral or may be one or more pharmaceutically acceptable salts, crystalline forms, non-crystalline forms, hydrates, or solvates, or a combination thereof. References to the compounds may refer to the neutral molecule, and/or those additional forms thereof collectively and individually from the context. Pharmaceutically acceptable salts of the compounds include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

### B. Pharmaceutical Formulations

Pharmaceutical formulations that contain one or more the AGB derivative(s) disclosed herein, in a form suitable for administration to a mammal, are disclosed. Typically, the compound(s) in the pharmaceutical formulation is present in an amount effective to ameliorate one or more symptoms associated with a cancer in a subject.

The pharmaceutical formulation containing the compound(s) may also include one more pharmaceutically acceptable carriers and/or one or more pharmaceutically acceptable excipients. For example, the pharmaceutical formulation may be in the form of a liquid, such as a solution or a suspension, and contain one or more the disclosed compounds in an aqueous medium and, optionally, one or more suitable excipients for the liquid formulation. Optionally, the pharmaceutical formulation is in a solid form, and contains one or more the disclosed compounds and one or more suitable excipients for a solid formulation.

### 1. Carriers and Excipients

The pharmaceutical formulation can contain one or more pharmaceutically acceptable carriers and/or one or more pharmaceutically acceptable excipients. Suitable pharmaceutically acceptable carriers and excipients are generally recognized as safe (GRAS), and may be administered to an individual without causing undesirable biological side effects or unwanted interactions.

Representative carriers and excipients that can be used in the pharmaceutical formulations include solvents (including buffers), diluents, pH modifying agents, preservatives, antioxidants, suspending agents, wetting agents, viscosity modifiers, tonicity agents, and stabilizing agents, and a combination thereof.

In some forms, the compounds can be dissolved or suspended in a suitable carrier to form a liquid pharmaceutical formulation, such as sterile saline, phosphate buffered saline (PBS), balanced salt solution (BSS), viscous gel, or other pharmaceutically acceptable carriers for administration. The pharmaceutical formulation may also be a sterile solution, suspension, or emulsion in a nontoxic, parenterally acceptable diluent or solvent.

Excipients can be added to a liquid or solid pharmaceutical formulation to assist in sterility, stability (e.g. shelf-life), integration, and to adjust and/or maintain pH or isotonicity of the compounds in the pharmaceutical formulation, such as diluents, pH modifying agents, preservatives, antioxidants, suspending agents, wetting agents, viscosity modifiers, tonicity agents, and stabilizing agents, and a combination thereof.

### 2. Form

The pharmaceutical formulation containing one or more the disclosed AGB derivative(s) can be in a liquid form or a solid form, such as a liquid formulation or a solid formulation for oral administration, mucosal administration, or parenteral administration (e.g. intramuscular administration, intravenous administration, intraperitoneal administration, and subcutaneous administration), or topical administration, to a subject.

a. Oral Formulations

In some forms, the pharmaceutical formulation containing one or more the disclosed compound(s) can be in a form suitable for oral administration to a subject, such as a mammal (i.e. an oral formulation). Oral administration may involve swallowing, so that the compound(s) enter the gastrointestinal tract, or buccal or sublingual administration may be employed by which the compound(s) enter(s) the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays, and liquid formulations.

Liquid formulations for oral administration include suspensions, solutions, syrups, and elixirs. Such oral formulations may be employed as fillers in soft or hard capsules and can contain one or more suitable carriers and/or excipients, for example, water, ethanol, polyethylene glycol, propylene glycol, chitosan polymers and chitosan derivatives (e.g. N-trimethylene chloride chitosan, chitosan esters, chitosan modified with hydrophilic groups, such as amino groups, carboxyl groups, sulfate groups, etc.), methylcellulose, a suitable oil, one or more emulsifying agents, and/or suspending agents. Liquid formulations for oral administration may also be prepared by the reconstitution of a solid, for example, from a sachet.

Optionally, the compound(s) is/are included in a fast-dissolving and/or fast-disintegrating dosage form.

For tablet or capsule dosage forms, in addition to the compound(s) described herein, tablets generally contain disintegrants, binders, diluents, surface active agents, lubricants, glidants, antioxidants, colourants, flavouring agents, preservatives, or taste masking agents, or a combination thereof.

Examples of suitable disintegrants for forming a table or capsule dosage form containing the compound(s) include, but are not limited to, sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant can have a concentration in a range from about 1 wt % to about 25 wt %, from about 5 wt % to about 20 wt % of the tablet or capsule dosage form containing the compound(s).

Binders are generally used to impart cohesive qualities to a tablet formulation. Suitable binders for forming a tablet or capsule formulation containing the compound(s) include, but are not limited to, microcrystalline cellulose, gelatin, sugars, polyethylene glycol, natural and synthetic gums, polyvinylpyrrolidone, pregelatinised starch, chitosan polymers and chitosan derivatives (e.g. N-trimethylene chloride chitosan, chitosan esters, chitosan modified with hydrophilic groups, such as amino groups, carboxyl groups, sulfate groups, etc.), hydroxypropyl cellulose, and hydroxypropyl methylcellulose.

Suitable diluents for forming a table or capsule formulation containing the compound(s) include, but are not limited to, lactose (as, for example, the monohydrate, spray-dried monohydrate or anhydrous form), chitosan polymers and chitosan derivatives (e.g. N-trimethylene chloride chitosan, chitosan esters, chitosan modified with hydrophilic groups, such as amino groups, carboxyl groups, sulfate groups, etc.), N-sulfonated derivatives of chitosan, quaternarized derivatives of chitosan, carbosyalkylated chitosan, microcrystalline chitosan, mannitol, xylitol, dextrose, sucrose, sorbitol, microcrystalline cellulose, starch and dibasic calcium phosphate dihydrate.

Tablet or capsule formulations containing the compound(s) may also contain surface active agents, such as sodium lauryl sulfate and polysorbate 80, and glidants such as silicon dioxide and talc. When present, surface active agents can have a concentration in a range from about 0.2 wt % to 5 wt % of the tablet or capsule formulation.

Tablet or capsule formulations containing the compound(s) also can contain lubricants, such as magnesium stearate, calcium stearate, zinc stearate, sodium stearyl fumarate, and mixtures of magnesium stearate with sodium lauryl sulphate. Lubricants can have a concentration in a range from about 0.25 wt % to 10 wt %, from about 0.5 wt % to about 3 wt % of the tablet or capsule formulation.

Other possible excipients included in a tablet or capsule formulation containing the compound(s) include glidants (e.g. Talc or colloidal anhydrous silica at about 0.1 wt % to about 3 wt % of the table or capsule formulation), antioxidants, colourants, flavouring agents, preservatives and taste-masking agents. When present, glidants can have a concentration in a range from about 0.2 wt % to 1 wt % of the tablet or capsule formulation.

An exemplary tablet formulation contains up to about 80 wt % of the compound(s) described herein, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet or capsule blends, including the compound(s) and one or more suitable excipients, may be compressed directly or by roller to form tablets. Tablet or capsule blends or portions of the blends may alternatively be wet-, dry-, or melt-granulated, melt congealed, or extruded before tableting. The final table or capsule formulation may contain one or more layers and may be coated or uncoated; it may even be encapsulated in a particle, such as a polymeric particle or a liposomal particle.

Solid formulations containing the compound(s) for oral administration may be formulated to be immediate and/or modified release. Modified release formulations include delayed, sustained, pulsed, controlled, targeted and programmed release formulations.

b. Parenteral Formulations

In some forms, the pharmaceutical formulation containing one or more the disclosed compounds can be in a form suitable for administration directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

For example, the pharmaceutical formulation containing one or more the compounds is in a form suitable for intramuscular administration, intravenous administration, intraperitoneal administration, or subcutaneous administration, or a combination thereof.

Parenteral formulations containing the compound(s) described herein are typically aqueous solutions which can contain excipients such as salts, carbohydrates and buffering agents (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4), but, for some applications, they may be more suitably formulated as a sterile aqueous solution or as a dried form to be used in conjunction with a suitable vehicle such as sterile, pyrogen-free water.

The liquid formulations containing the compound(s) for parenteral administration may be a solution, a suspension, or an emulsion.

The liquid pharmaceutically acceptable carrier forming the parenteral formulation containing the compound(s) can include one or more physiologically compatible buffers, such as a phosphate buffers. One skilled in the art can readily determine a suitable saline content and pH for an aqueous carrier for administration (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4).

Liquid formulations containing the compound(s) for parenteral administration may include one or more suspending agents, such as cellulose derivatives, sodium alginate, polyvinylpyrrolidone, gum tragacanth, or lecithin. The liquid formulations may also include one or more preservatives, such as ethyl or n-propyl p-hydroxybenzoate.

In some forms, the liquid formulation containing the compound(s) contains one or more solvents that are low toxicity organic (i.e., nonaqueous) class 3 residual solvents, such as ethanol, acetone, ethyl acetate, tetrahydofuran, ethyl ether, and propanol, and a combination thereof. Any such solvents included in the liquid formulation should not detrimentally react with the compound(s) and any additional active agents when present in the liquid formulation. Solvents such as freon, alcohol, glycol, polyglycol, or fatty acid, can also be included in the liquid formulation containing the compound(s) as desired to increase the volatility of the solution or suspension.

Liquid formulations containing the compound(s) for parenteral administration may also contain minor amounts of polymers, surfactants, or other pharmaceutically acceptable excipients known to those in the art. In this context, “minor amounts” means an amount that is sufficiently small to avoid adversely affecting uptake of the compounds by the targeted cells, such as pituitary gonadotrophs.

The preparation of parenteral formulations containing the compound(s) is typically under sterile conditions, for example, by lyophilisation, which can be accomplished using standard pharmaceutical techniques known to those skilled in the art.

Formulations for parenteral administration containing the compound(s) may be formulated to provide immediate and/or modified release of the active agent. Modified release formulations include delayed, sustained, pulsed, controlled, targeted and programmed release formulations.

c. Pulmonary and Mucosal Formulations

In some forms, the pharmaceutical formulation containing one or more the disclosed compounds can be in a form suitable for pulmonary or mucosal administration. The administration can include delivery of the composition to the lungs, nasal, oral (sublingual, buccal), vaginal, or rectal mucosa.

For example, the compounds can be administered intranasally or by oral inhalation, such as in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (such as an atomiser using electrohydrodynamics to produce a fine mist), or nebuliser, with or without the use of a suitable propellant, such as water, ethanol-water mixture, 1,1,1,2-tetrafluoroethane or 1,1,1,2,3,3,3-heptafluoropropane. For intranasal or oral inhalation use, the powder may comprise a bioadhesive agent, for example, chitosan or cyclodextrin. The term aerosol as used herein refers to any preparation of a fine mist of particles, which can be in solution or a suspension, whether or not it is produced using a propellant. Aerosols can be produced using standard techniques, such as ultrasonication or high-pressure treatment.

The pressurized container, pump, spray, atomizer, or nebuliser contains a solution or suspension of one or more of the compounds including, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, a drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compounds described herein, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose, and trehalose.

A suitable solution formulation containing the compound(s) for use in an atomizer using electrohydrodynamics to produce a fine mist may contain from 1 μg to 20 mg of one or more of the compounds per actuation and the actuation volume may vary from 1 μl to 100 μl. A typical formulation may contain one or more of the compounds described herein, propylene glycol, sterile water, ethanol and sodium chloride. Alternative solvents that may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the compounds are typically arranged to administer a metered dose or “puff.” The overall daily dose will be administered in a single dose or, more usually, as divided doses throughout the day.

In some forms, the compounds can be formulated for pulmonary delivery, such as intranasal administration or oral inhalation. Carriers for pulmonary formulations can be divided into those for dry powder formulations and for administration as solutions. For administration via the upper respiratory tract, the formulation can be formulated into an aqueous solution, e.g., water or isotonic saline, buffered or un-buffered, or as an aqueous suspension, for intranasal administration as drops or as a spray. Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

In some forms, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In some forms, solvents that are low toxicity organic (i.e. nonaqueous) class 3 residual solvents, such as ethanol, acetone, ethyl acetate, tetrahydrofuran, ethyl ether, and propanol may be used for the formulations containing the compound(s). The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In some forms, the pharmaceutical formulations containing the compound(s) may contain minor amounts of polymers, surfactants, or other excipients well known to those of the art. In this context, “minor amounts” means no excipients are present that might affect or mediate uptake of the compounds by cells and that the excipients that are present in amount that do not adversely affect uptake of compounds by cells.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Non-aqueous suspensions of lipids can also be prepared in absolute ethanol using a reusable PARI LC Jet+ nebulizer (PARI Respiratory Equipment, Monterey, CA).

d. Topical Formulations

The compounds can be administered directly to the external surface of the skin or the mucous membranes (including the surface membranes of the nose, lungs and mouth), such that the compounds can cross the external surface of the skin or mucous membrane and enters the underlying tissues.

Formulations for topical administration generally contain a dermatologically acceptable carrier that is suitable for application to the skin, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, salts, and the like). Examples of topical carrier systems useful in the present formulations are described in the following four references all of which are incorporated herein by reference in their entirety: “Sun Products Formulary” Cosmetics & Toiletries, vol. 105, pp. 122-139 (December 1990); “Sun Products Formulary,” Cosmetics & Toiletries, vol. 102, pp. 117-136 (March 1987); U.S. Pat. No. 5,605,894 to Blank et al., and U.S. Pat. No. 5,681,852 to Bissett.

Formulations containing the compound(s) for topical administration may be formulated to be immediate and/or modified release. Modified release formulations include delayed, sustained, pulsed, controlled, targeted and programmed release formulations. Thus, the compounds may be formulated as a solid, semi-solid, or thixotropic liquid for administration as an implanted depot providing modified release of the compounds. Examples of such formulations include drug-coated stents and poly(dl-lactic-coglycolic)acid (PGLA) microspheres.

### 3. Additional Active Agent(s)

In some forms, the pharmaceutical formulation can include one or more additional active agents that are different from the AGB derivatives, such as one or more additional anticancer agents. Anticancer agents that can be included in the pharmaceutical compositions or formulations are known, for example, see the National Cancer Institute database, “A to Z List of Cancer Drugs,” website cancer.gov/about-cancer/treatment/drugs.

Exemplary anticancer drugs that can be included in the pharmaceutical formulation containing the compound(s) include, but are not limited to, olaparib, abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); rituximab, cyclophosphamide, doxorubicin, vincristine, prednisolone (RCHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

### 4. Dosages/wt % Concentration

In some forms, the pharmaceutical formulation contains an effective amount of the compound(s) for ameliorating one or more symptoms associated with a cancer in a subject.

In some forms, the compound(s) is present in the pharmaceutical formulation in an effective amount to induce apoptosis of cancer cells in the subject. The cytotoxicity of the compounds against cancer cells can be evaluated using the IC₅₀ values against the cancer cells, measured using known methods, such as MTT assay. Whether apoptosis of cancer cells is induced in the subject may be identified by a change of at least 5% in relevant biomarker or gene expression profile in a biological sample of the subject, compared to the biomarker or gene expression profile in the biological sample of the subject before treatment with the compound(s). Exemplary biomarkers for showing apoptosis of cancer cells includes, but are not limited to, HER2 for breast cancer; PSA for prostate cancer; carcinoembryonic antigen (CEA), squamous cell carcinoma antigen (SCC), neuron-specific enolase (NSE), cytokeratin 19 fragment (CYFRA), and pro-gastrin-releasing peptide (proGRP) for lung cancer, or others. Alternatively or additionally, whether apoptosis of cancer cells is induced may be identified by chromatin condensation and fragmentation; and/or cleavalges of PARP, caspase-9, and/or caspase-3. In some forms, the pharmaceutical formulation contains the compound(s) in an amount that is effective to induce apoptosis of cancer cells as shown by chromatin condensation and fragmentation; cleavalges of PARP, caspase-9, and/or caspase-3; and/or changes in the levels of proteins involved in apoptotic signaling using Western blotting.

In some forms, the compound(s) is present in the pharmaceutical formulation in an effective amount to inhibit the growth of a tumor by at least 40%, at least 50%, or at least 55% in the subject compared to the same tumor in a control subject. Inhibition of tumor can be determined by known methods, such as by measuring the size of an isolated solid tumor or measuring the tumor size in vivo using imaging. In some forms, the pharmaceutical formulation contains the compound(s) in an amount that is effective to inhibit the growth of a tumor by at least 40%, at least 50%, or at least 55% in the subject, compared to the same tumor in a control subject by the end of a monitoring time period, such as by the end of 21 days, 28 days, 31 days, 2 months, 3 months, 6 months, or a year, optionally without any toxicity as shown by a stable body weight of the subject having the tumor. A stable body weight refers to a body weight change of less than about 10% during and by the end of the monitoring time period.

The total concentration of the compound(s) in the pharmaceutical formulation can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %. The term “total concentration of the compound(s) in the pharmaceutical formulation” refers to the sum of the weight of all compounds(s) relative to the weight of the formulation.

In some forms, the total concentration of the compound(s) in the pharmaceutical formulation that is effective to ameliorate one or more symptoms associated with a cancer in a subject, can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

In some forms, the total concentration of the compound(s) in the pharmaceutical formulation that is effective to induce apoptosis of cancer cells in the subject can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

In some forms, the total concentration of the compound(s) in the pharmaceutical formulation that is effective to inhibit the growth of a tumor by at least 40%, at least 50%, or at least 55% in the subject compared to the same tumor in a control subject, by the end of a monitoring period, can be at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

In some forms, the pharmaceutical formulation containing the compound(s) can be provided in a unit dosage form. The dosage of the compounds in the pharmaceutical formulation in the unit dosage form can be in a range from about 0.002 mg to about 1 mg, in a range from about 0.006 mg to about 0.6 mg, in a range from about 0.01 mg to about 0.4 mg, in a range from about 0.02 mg to about 0.3 mg, or in a range from about 0.01 mg to about 0.2 mg.

### III. Methods for Synthesis of AGB Derivatives

Methods for synthesizing AGB derivatives containing sulfone moiety have been developed. Prior to the disclosed methods, synthesis of sulfone-cooperated AGB and intermediates thereof is either absent or overly complex and lengthy. The methods disclosed herein for the synthesis of AGB derivatives employ easy-to-follow synthetic paths using commercially available and easy-to-handle reagents.

Generally, the methods include (1) converting a starting reagent to a sulfone intermediate or a ketone/sulfone intermediate; and (2) converting the sulfone intermediate or ketone/sulfone intermediate to the compound. The sulfone intermediate may contain an aryl sulfone or an alkyl sulfone. In some forms, a ketone can be installed on the sulfone intermediate to form the ketone/sulfone intermediate.

In some forms, the sulfone intermediate is an aryl sulfone intermediate, which may be prepared in a single step. For example, the sulfone intermediate produced in step (1) is an aryl sulfone intermediate that is prepared following a direct Friedel-Crafts sulfonylation reaction. This reaction can be applied to aryl with both electron-neutral and electron-donating groups and excludes an oxidation process that is required in previous methods. For example, the synthesis described in H. Burton and E. Hoggarth, Journal of the Chemical Society, 1945, 468-470 (“Burton”) applies to only electron-withdrawing systems and suffers from a multi-step process, including protection/deprotection of phenols and oxidation using H₂O₂. Further, Burton uses a disulfide as the starting material, which had limited availability. In contrast, in some forms, the disclosed methods can use a one-step Friedel-Crafts sulfonylation to install an aryl sulfone, which may carry electron-neutral and electron-donating groups, directly onto an aryl ring, without any protection/deprotection of phenols and without oxidation involving oxidants. Further, this simple reaction uses readily available starting material.

In some forms, the sulfone intermediate is an alkyl sulfone intermediate using a sulfur dioxide surrogate to introduce the sulfonyl (—SO₂—) group, producing lithium sulfinate (RSO₂Li) as a versatile intermediate, which then reacts with an alkyl electrophile to produce the alkyl sulfone intermediate. In these forms, the reactions for producing the alkyl sulfone intermediate omits the use of disulfide or H₂O₂. In these forms, a deprotection step may be further preformed with a suitable agent, such as BBr₃, to convert one or more —O-alkyl group to —OH.

More specific methods for synthesizing exemplary AGB derivatives are described in the Examples below.

### A. Converting Starting Reagent to Sulfone Intermediate

The methods for synthesizing AGB derivatives includes a first step of converting a starting reagent to a sulfone intermediate or a ketone/sulfone intermediate.

In some forms, the starting reagent can have the structure of:

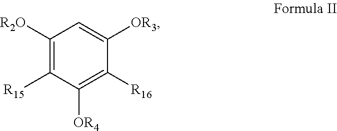

the sulfone intermediate can have the structure of:

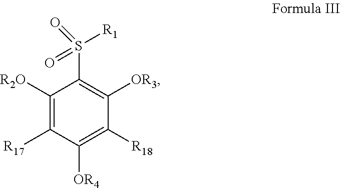

the ketone/sulfone intermediate can have the structure of:

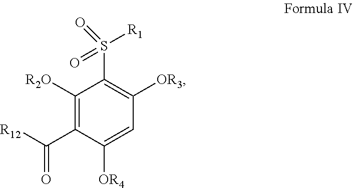

wherein: (i) R₁ can be an alkyl or

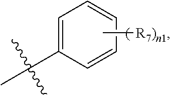

n1 can be an integer from 1 to 5, and R₇ can be hydrogen, an alkyl, —O-alkyl, hydroxyl, or —NR′₁R′₂, wherein R′₁ and R′₂ can be independently hydrogen or an alkyl; (ii) R₂, R₃, and R₄ can be independently hydrogen or an alkyl; (iii) R₁₅ and R₁₆ can be independently hydrogen or an alkyl; (iv) R₁₇ and R₁₈ can be independently hydrogen, an alkyl, or

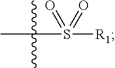

and (v) R₁₂ can be hydrogen, hydroxyl, an alkyl, or —O-alkyl.

Typically, at least one of R₁₅ and R₁₆ is hydrogen; and/or at least one of R₁₇ and R₁₈ is hydrogen. In some forms, at least one of R₁₅ and R₁₆ is hydrogen, and correspondingly, at least one of R₁₇ and R₁₈ is hydrogen. For example, R₁₅ and R₁₇ are hydrogen, or R₁₆ and R₁₈ are hydrogen. For example, R₁₅-R₁₈ are hydrogen.

### 1. Converting Starting Reagent to Aryl Sulfone Intermediate

In some forms, in the first step (i.e., step (1)), an aryl sulfone intermediate is formed, where R₁ of Formula III can be a substituted or unsubstituted phenyl, such as any of those described above in Section II(A). For example, R₁ can be

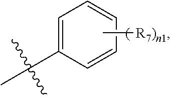

where n1 can be an integer from 1 to 5, R₇ can be hydrogen, an electron neutral group, or an electron donating group. For example, R₇ can be hydrogen, an alkyl, —O-alkyl, hydroxyl, or —NR′₁R′₂, wherein R′₁ and R′₂ can be independently hydrogen or an alkyl.

In these forms, the first conversion step (1) can be achieved by (a) heating a first reaction mixture at a suitable temperature for a suitable period of time to form the aryl sulfone intermediate. The first reaction mixture contains the starting reagent and a sulfonyl halide, and optionally a suitable organic solvent, such as 1,2-dichloroethane or nitrobenzene, or a mixture thereof.

In some forms, the sulfonyl halide used in the first reaction mixture can have the structure of

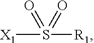

where X₁ can be a halide (e.g., chloride, bromide, iodide, or fluoride). Typically, the starting reagent and sulfonyl halide used in the first reaction mixture are readily available, for example, can be obtained from a commercial source.

Typically, when an aryl sulfone intermediate is the target product of the first step, the aryl sulfone intermediate is formed using a one-step reaction between the starting reagent and sulfonyl halide, such as a direct Friedel-Crafts sulfonylation reaction. Using this reaction, sulfonyl halides that carry an aryl with electron-neutral and/or electron-donating groups can be directly installed on the aryl ring of the starting reagent of Formula II to form the aryl sulfone intermediate.

Generally, the reaction in step (1) can be performed at a temperature up to 100° C., up to 90° C., up to 80° C., in a range from 40° C. to 100° C., from 50° C. to 100° C., from 60° C. to 100° C., or from 70° C. to 100° C., at 1 atm, such as about 80° C., at 1 atm; for a time period up to 12 hours, up to 10 hours, up to 8 hours, or up to 6 hours, in a range from about 1 hour to about 12 hours, from about 1 hour to about 10 hours, from about 1 hour to about 8 hours, or from about 1 hour to about 6 hours, such as about 3 hours; and optionally in the presence of a suitable catalyst, such as AlCl₃.

More specific conditions and reagents for the reaction to form exemplary aryl sulfone intermediate in step (1) are described in the Examples below.

### 2. Converting Starting Reagent to Alkyl Sulfone Intermediate

In some forms, in the first step (i.e., step (1)), an alkyl sulfone intermediate is formed, where R₁ of Formula III can be an alkyl, such as any of those described above in Section II(A). In these forms, the alkyl sulfone intermediate can be obtained by (a) converting the starting reagent to a lithium sulfinate intermediate; and (b) converting the lithium sulfinate intermediate to the sulfone intermediate.

In some forms, the lithium sulfinate intermediate can have the structure of:

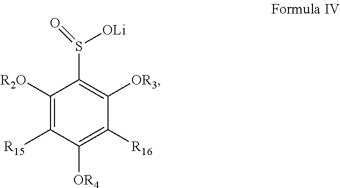

wherein: (i) R₂, R₃, and R₄ can be independently hydrogen or an alkyl; (ii) R₁₅ and R₁₆ can be independently hydrogen or an alkyl. In some forms, at least one of R₁₅ and R₁₆ is hydrogen. For example, R₁₅ is hydrogen or R₁₆ is hydrogen. For example, R₁₅ and R₁₆ are hydrogen.

In some forms, step (a) converting the starting reagent to a lithium sulfinate intermediate can be achieved by mixing the starting reagent, an organolithium reagent, and a sulfur dioxide surrogate reagent to form the lithium sulfinate intermediate.

In step (a), the organolithium reagent and sulfur dioxide surrogate reagent may be added to a solution of the starting reagent simultaneously or in a stepwise manner. For example, the organolithium reagent is first added to a solution of the starting reagent and mixed at a suitable temperature (e.g., about 40° C., at 1 atm) for a suitable period of time (e.g., a time period from about 1 hour to about 10 hours or from about 1 hour to about 5 hours, such as about 3 hours); then the sulfur dioxide surrogate is added to the mixture and mixed a suitable temperature (e.g., room temperature at 1 atm) for a suitable period of time (e.g., a time period from about 10 mins to about 2 hours or from about 20 mins to about 1 hour, such as about 30 mins) to form the lithium sulfinate intermediate.

In some forms, the organolithium reagent used in step (a) can be an alkyl-lithium (e.g., n-butyllithium). In some forms, the sulfur dioxide surrogate used in step (a) can have the structure of:

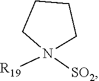

where R₁₉ can be an alkyl (e.g., methyl).

Typically, in step (a), the sulfur dioxide surrogate can introduce a sulfonyl (—SO₂—) group onto the aryl ring of the starting material, producing lithium sulfinate (RSO₂Li) as a versatile intermediate that can react with a variety of alkyl electrophiles in step (b) to form the alkyl sulfone intermediate.

In some forms, step (b) converting the lithium sulfinate intermediate to the sulfone intermediate can be achieved by mixing the lithium sulfinate intermediate with an alkyl electrophile to form the sulfone intermediate. The mixing in step (b) can be performed at a suitable temperature for a suitable period of time, such as room temperature at 1 atm for a time period from about 5 hours to about 24 hours, from about 10 hours to about 20 hours, from about 10 hours to about 16 hours, or from about 10 hours to about 12 hours.

In some forms, the alkyl electrophile used in step (b) can be alkyl-X₂, where X₂ can be a halide (e.g., e.g., chloride, bromide, iodide, or fluoride).

Optionally, following step (b), a deprotection step can be preformed with a suitable agent, such as BBr₃, to convert one or more —O-alkyl group(s) on the alkyl sulfone intermediate to —OH.

More specific conditions and reagents for the reactions to form exemplary alkyl sulfone intermediate in step (1) are described in the Examples below.

### 3. Converting Sulfone Intermediate to Ketone/Sulfone Intermediate

In some forms, the target product in step (1) converting a starting reagent to a sulfone intermediate or a ketone/sulfone intermediate is a ketone/sulfone intermediate, such as the ketone/sulfone intermediate of Formula IV.

In these forms, a ketone can be installed on a sulfone intermediate, such as the aryl sulfone intermediate or alkyl sulfone intermediate formed using the reactions/processes described above, to form the ketone/sulfone intermediate.

For example, following the formation of aryl sulfone intermediate or alkyl sulfone intermediate using the reactions/processes described above, a step (c) heating a second reaction mixture at a suitable temperature for a suitable period of time is performed to form the ketone/sulfone intermediate. The second reaction mixture contains the aryl sulfone intermediate or alkyl sulfone intermediate, and a ketone reagent, and optionally a suitable organic solvent (e.g., BF₃·Et₂O). In some forms, the ketone reagent can be R₁₂—C(O)—X₃, where R₁₂ can be hydrogen, hydroxyl, an alkyl, or —O-alkyl, and X₃ can be a halide (e.g., e.g., chloride, bromide, iodide, or fluoride).

Generally, the reaction in step (c) can be performed at a temperature up to 100° C., up to 90° C., up to 80° C., in a range from 40° C. to 100° C., from 50° C. to 100° C., from 60° C. to 100° C., or from 70° C. to 100° C., at 1 atm, such as about 80° C., at 1 atm; for a time period up to 12 hours, up to 10 hours, up to 8 hours, or up to 6 hours, in a range from about 1 hour to about 12 hours, from about 1 hour to about 10 hours, from about 1 hour to about 8 hours, or from about 1 hour to about 6 hours, such as about 3 hours.

More specific conditions and reagents for the reactions to form exemplary ketone/sulfone intermediate in step (1) are described in the Examples below.

### B. Converting Sulfone Intermediate or Ketone/Sulfone Intermediate to AGB Derivatives

The methods for synthesizing AGB derivatives includes a second step of converting the sulfone intermediate or ketone/sulfone intermediate to form the AGB derivatives. In the second conversion step (2), the sulfone intermediate or ketone/sulfone intermediate can couple with a ring, typically a readily available aryl reagent, to produce the AGB derivatives.

The second conversion step (2) can be achieved by (d) heating a third reaction mixture at a suitable temperature for a suitable period of time to form the compound. The third reaction mixture contains the sulfone intermediate or ketone/sulfone intermediate formed in step (1) described above, and the aryl reagent.

In some forms, the aryl reagent can have the structure of

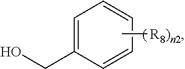

where n2 can be an integer from 1 to 5, R₈ can be hydrogen, hydroxyl, an alkyl, —O-alkyl, or —C(O)—R₉, and R₉ can be hydrogen, hydroxyl, an alkyl, or —O-alkyl.

In some forms, the aryl reagent can have the structure of

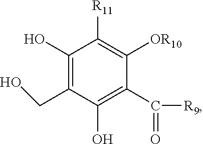

where R₁₀ can be hydrogen or an alkyl, and R₉ and R₁₁ can be independently hydrogen, hydroxyl, or an alkyl. In some forms, R₉-R₁₁ can be independently an alkyl, such as methyl, ethyl, n-propyl, isopropyl, n-butyl, isobutyl, tert-butyl, cyclopentyl, cyclohexyl, etc.

In some forms, the aryl reagent can be

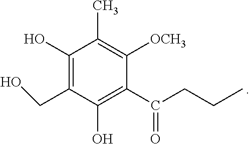 [SMILES: CCCC(=O)c1c(O)c(CO)c(O)c(C)c1OC]

Generally, the reaction in step (2) can be performed at a temperature up to 150° C., up to 120° C., up to 100° C., in a range from 50° C. to 150° C., from 60° C. to 120° C., from 80° C. to 120° C., or from 90° C. to 110° C., at 1 atm, such as about 100° C., at 1 atm; for a time period up to 5 hours, up to 3 hours, up to 2 hours, or up to 1 hour, in a range from about 20 mins to about 5 hours or from about 30 mins to about 2 hours, such as about 1 hour; and optionally in the presence of a suitable catalyst, such as ZnCl₂.

More specific conditions and reagents for coupling exemplary sulfone intermediates and ketone/sulfone intermediates with an exemplary aryl reagent to form exemplary AGB derivatives are described in the Examples below.

### C. Optional Steps

The disclosed methods can include one or more optional steps, such as quenching reactions with a suitable quenching agent subsequent to step (a), step (b), and/or step (c); and/or purifying the sulfone intermediate, ketone/sulfone intermediate, and/or AGB derivative, optionally by extraction and/or flash column chromatography.

### 1. Quenching Reactions

Optionally, upon completion of the reactions in step (a), step (b), and/or step (c), a quenching agent is added to the respective reaction mixture to quench the reaction.

The specific quenching agent can be selected based on the specific reaction. For example, to quench the Friedel-Crafts sulfonylation reaction forming the aryl sulfone intermediate, an acid solution (e.g., concentrated HCl solution) is added to the reaction mixture. For example, to quench the reaction forming the alkyl sulfone intermediate, water/brine is added to the reaction mixture. For example, to quench the reaction forming the sulfone/ketone intermediate, a KOAc solution is added to the reaction mixture. For example, to quench the reaction forming the AGB derivative, water/brine is added to the reaction mixture.

2. Purifying Intermediates and/or AGB Derivatives

Optionally, the disclosed method includes a step of purifying the sulfone intermediate, ketone/sulfone intermediate, and/or AGB derivative to remove impurities, such as unreacted reactants, in the reaction product, and thereby obtain isolated intermediates and/or AGB derivatives, subsequent to each reaction. The purification can be performed using known methods, such as using extraction, flash column chromatography on silica gel, and/or recrystallization.

### IV. Methods for Using the AGB Derivatives

In some forms, the disclosed AGB derivatives have anticancer properties and thereby can be used in methods for treating a cancer in a subject in need thereof. In some forms, the AGB derivatives can be used in methods for treating cancer cells in a subject in need thereof.

It will be appreciated the disclosed methods can be methods of treatment of the symptoms and conditions described herein. “Treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### A. Treating Cancer

Methods of using the AGB derivatives for treating a cancer in a subject in need thereof are disclosed. Generally, the method includes (i) administering to the subject a pharmaceutical formulation containing one or more of the AGB derivatives described above. The administration step can occur one or more times.

The subject can be a mammal, such as a human, a dog, a cat, a rat, a monkey, rabbits, guinea pigs, etc., that is in need of cancer treatment. In some forms, the subject can be exhibiting symptoms of or diagnosed with cancer.

The pharmaceutical formulation can be administered by oral administration, parenteral administration, mucosal administration, or topical administration, or a combination thereof. The compound(s) can be administered by a medical professional or the subject being treated (e.g. self-administration).

### 1. Cancers

The cancer being treated using the disclosed methods can be tumors, such as tumors of the hematopoietic and lymphoid tissues or hematopoietic and lymphoid malignancies, tumors that affect the blood, bone marrow, lymph, and lymphatic system, and tumors located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax, and genito-urinary apparatus.

In some forms of the method, the cancer can be a lung cancer, such as a large cell lung cancer.

In some forms of the method, the cancer can be a colon cancer, breast cancer, ovarian cancer, cervical cancer, lung cancer, rectal cancer, kidney cancer, liver cancer, brain cancer, or leukemia, or a combination thereof. In some forms of the method, the cancer can be breast cancer, such as triple negative breast cancer (TNBC).

In some forms of the method, the cancer can be AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system lymphoma, central nervous system lymphoma, cerebellar astrocytoma, brain astrocytoma, cancer of the cervix, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood brain astrocytoma, childhood extracranial germ cell tumors, childhood Hodgkin's disease, childhood Hodgkin's lymphoma, childhood visual pathway and hypothalamic glioma, childhood lymphoblastic leukemia, childhood medulloblastoma, childhood non-Hodgkin's lymphoma, childhood supratentorial primitive neuroectodermal and pineal tumors, childhood primary liver cancer, childhood rhabdomyosarcoma, childhood soft tissue sarcoma, childhood visual pathway and hypothalamic glioma, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the esophagus, Ewing's sarcoma and related tumors, cancer of the exocrine pancreas, extracranial germ cell tumor, extragonadal germ cell tumor, extrahepatic biliary tract cancer, cancer of the eye, breast cancer in women, Gaucher's disease, cancer of the gallbladder, gastric cancer, gastrointestinal carcinoid tumor, gastrointestinal tumors, germ cell tumors, gestational trophoblastic tumor, tricoleukemia, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, cancer of kidney, cancer of the larynx, cancer of the lip and mouth, cancer of the liver, cancer of the lung, lymphoproliferative disorders, macroglobulinemia, breast cancer in men, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma during pregnancy, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, phaeochromocytoma, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

### 2. Effective Amount/Dosage

Generally, following the administration step of the disclosed method, the pharmaceutical formulation is administered in an effective amount to ameliorate one or more symptoms associated with a cancer in a subject.

For example, the method includes only a single administration of the pharmaceutical formulation, wherein following the administration step, an effective amount of the compound(s) to ameliorate one or more symptoms associated with the cancer in the subject is administered to the subject.

For example, the method includes more than one step of administering to the subject the pharmaceutical formulation, wherein following all of the administration steps, an effective amount of the compound(s) to ameliorate one or more symptoms associated with the cancer in the subject is administered to the subject.

In some forms, following the administration step or all of the administration steps (when more than one administration is performed), the pharmaceutical formulation is administered in an effective amount to inhibit the growth of a tumor by at least 40%, at least 50%, or at least 55% in the subject compared to the same tumor in a control subject or compared to the subject before administration of the pharmaceutical formulation. Whether tumor growth is inhibited may be identified by a variety of diagnostic manners known to one skill in the art including, but not limited to, observation of the reduction in size or number of tumor masses, in comparison with the same type of tumor in a control subject, using imaging, by the end of a certain time period following administration of the effective amount of pharmaceutical formulation. Imaging suitable for measuring the size or number of tumor masses include, but are not limited to, transrectal ultrasound, MRI, computerized tomography (“CT”) scan, positron emission tomography (“PET”) imaging, multiparametric ultrasound (“US”), or a combination thereof, for example, PET-CT, PET-MRI, MRI-US, etc.

In some forms, following the administration step or all of the administration steps (when more than one administration is performed), the pharmaceutical formulation is administered in an effective amount to reduce the level of a biomarker associated with a cancer in the blood of the subject by at least 5%, at least 10%, at least 15%, at least 20%, at least 25%, or at least 30% compared to the level of the biomarker in the blood of the subject before treatment. For example, the pharmaceutical formulation is administered in an effective amount to reduce the level of a biomarker associated with a lung cancer (e.g. carcinoembryonic antigen (CEA), squamous cell carcinoma antigen (SCC), neuron-specific enolase (NSE), cytokeratin 19 fragment (CYFRA), and pro-gastrin-releasing peptide (proGRP)) in the blood of the subject by at least 5%, at least 10%, at least 15%, at least 20%, at least 25%, or at least 30% compared to the level of the biomarker in the blood of the subject before treatment.

Administering an effective amount of the pharmaceutical formulation can be achieved in a single administration step or using multiple administration steps. For example, if the unit dosage form contains an effective amount of the compound(s) to inhibit the growth of a tumor by at least 40%, at least 50%, or at least 55% in the subject and/or reduce the level of a biomarker associated with a cancer in the blood of the subject, then the method only requires a single administration step. Alternatively, if the unit dosage form contains less than the required effective amount of the compound(s) to inhibit the growth of a tumor by at least 40%, at least 50%, or at least 55% in the subject and/or induce apoptosis of cancer cells, then the method involves at least two steps of administering the pharmaceutical formulation, and optionally more than two steps of administering the pharmaceutical formulation to the subject until an effective amount of the pharmaceutical formulation is administered to the subject to inhibit the growth of a tumor by at least 40%, at least 50%, or at least 55% in the subject and/or to reduce the level of a biomarker associated with a cancer in the blood of the subject. When multiple administration steps are needed to administer an effective amount of the pharmaceutical formulation to the patient, each administration step may administer the same dosage or different dosages of the pharmaceutical formulation to the patient.

When multiple administration steps are needed to administer an effective amount of the pharmaceutical formulation to the patient, the administration steps may be performed regularly or irregularly. For example, the administration steps are performed at a suitable frequency, such as every hour, every 2 hours, every 5 hours, every 8 hours, every day, every 2 days, every 3 days, every 5 days, every 7 days, every 10 days, every two weeks, or every month. For example, the administration step is performed every hour, every 2 hours, every 5 hours, every 8 hours, every day, every 2 days, every 3 days, every 5 days, every 7 days, every 10 days, every two weeks, or every month for a period between one day and 6 months, between one day and 3 months, between one and thirty days, between one and ten days, between one and three days, between one and two days, or for one day. Alternatively, the administration may be performed irregularly, for example, the administration step is performed 1 day after the first administration, then 2 days after the second administration, then 5 days after the third administration, then 7 days after the fourth administration, and then 30 days after the fifth administration. The time interval between administrations is determined based on the patient's needs.

In some forms, following a single administration or multiple administrations, the effective amount of compound(s) that is administered to the subject to ameliorate one or more symptoms associated with a cancer in a subject, can be in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 3 mg/kg of the subject.

### 3. Optional Steps

a. Administering Additional Active Agent(s)

One or more active agents in addition to the compounds may be administered to the subject throughout the method or at different intervals during the method. For example, the one or more additional active agents is administered to the subject prior to, during, and/or subsequent to step (i). In some forms, the one or more additional active agents can be included in a pharmaceutical formulation containing the compound(s) and is administered to the subject simultaneously with the compound(s) in the pharmaceutical formulation in association with one or more pharmaceutically acceptable excipients. In some forms, the one or more additional active agents can be administered separately from the pharmaceutical formulation containing the compound(s).

In some forms, the one or more additional active agents are one or more anticancer agents described above. The amount of the one or more additional anticancer agents required will vary from subject to subject according to their need.

### B. Treating Cancer Cells

In some forms, the compounds can be used in a method for treating cancer cells in a subject in need thereof.

The method can follow the method step described above, for example, administering to the subject the pharmaceutical formulation containing the compound(s), such as by oral administration, parenteral administration, mucosal administration, or topical administration, or a combination thereof. The administration step can occur one or more times to administer an effective amount of the compound(s) in the pharmaceutical formulation to kill cancer cells, depending on whether a unit dosage contains an effective amount of the compound(s) to kill the cancer cells. When multiple administrations are needed to achieve the required effective amount of the compound(s) in the subject, the dosage and frequency for each administration can follow the method described above.

In some forms, the method can include the additional step described above. For example, the user can administer one or more additional active agents to the subject prior to, during, and/or subsequent to administering the compound to the subject.

In some forms of the method, the compound(s) can have an IC₅₀ value against the cancer cells compared to an IC₅₀ value of the parent AGB, tested under the same condition (e.g., concentration in μM on the same order of magnitude).

In some forms of the method, the compound(s) can have an IC₅₀ value against the cancer cells lower than an IC₅₀ value of the same compound against non-cancerous cells, tested under the same condition.

The cancer cells being treated in the subject can be the cancer cells of any one of the cancers described above. For example, the cancer cells can be MDA-MB-231, MCF-7, HepG2, Hela, AGS, HTC116, SW480, SUNE-1, H460 (e.g. NCI-H460, stem-like NCI-H460, etc.), HCC827, H1650, or A549, or a combination thereof. In some forms, the cancer cells can be NCI-H460.

When comparing the IC₅₀ values of the compound against cancer cells with the IC₅₀ values of the same compound against non-cancerous cells, the non-cancerous cells can be from any normal tissue of the subject, such as CCD-19Lu.

### 1. Effective Amount/Dosage

Generally, following the administration step of the disclosed method, the pharmaceutical formulation is administered in an effective amount to induce apoptosis of cancer cells in a subject, compared to the subject before administered with the pharmaceutical formulation.

In some forms, following a single administration or multiple administrations, the effective amount of compound(s) that is administered to the subject to induce apoptosis of cancer cells in the subject, compared to the subject before administered with the pharmaceutical formulation, can be in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 3 mg/kg of the subject.

The disclosed compounds and methods can be further understood through the following enumerated paragraphs.
Paragraph 1. A compound having the structure of:

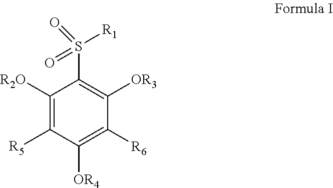

wherein:
   (i) R₁ is an alkyl (e.g., methyl, ethyl, n-propyl, isopropyl, n-butyl, isobutyl, tert-butyl, cyclopentyl, cyclohexyl, etc., for example, methyl, n-propyl, isopropyl, isobutyl, or cyclohexyl) or

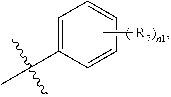

wherein
   n1 is an integer from 1 to 5, and R₇ is hydrogen, an alkyl, —O-alkyl, hydroxyl, or —NR′₁R′₂, wherein R′₁ and R′₂ are independently hydrogen or an alkyl;
   (ii) R₂, R₃, and R₄ are independently hydrogen or an alkyl; and
   (iii) R₅ and R₆ are independently an alkyl, —C(O)—R₁₂, or

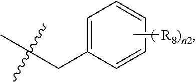

wherein
   n2 is an integer from 1 to 5,
      R₈ is hydrogen, hydroxyl, an alkyl, —O-alkyl, or —C(O)—R₉,
      R₉ and R₁₂ are independently hydrogen, hydroxyl, an alkyl, or —O-alkyl, and
      at least one of R₅ and R₆ is

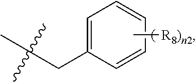

Paragraph 2. The compound of paragraph 1, wherein the

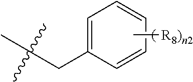

is

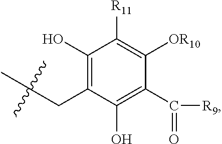

R₁₀ is hydrogen or an alkyl, and R₉ and R₁₁ are independently hydrogen, hydroxyl, or an alkyl.
Paragraph 3. The compound of paragraph 2, wherein R₉-R₁₁ are independently an alkyl (e.g., methyl, ethyl, n-propyl, isopropyl, n-butyl, isobutyl, tert-butyl, cyclopentyl, or cyclohexyl).
Paragraph 4. The compound of any one of paragraphs 1-3, wherein the

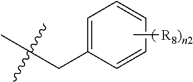

is

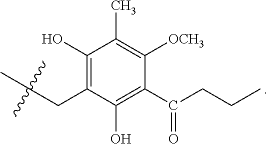 [SMILES: *Cc1c(O)c(C)c(OC)c(C(=O)CCC)c1O]

Paragraph 5. The compound of any one of paragraphs 1-4, wherein the

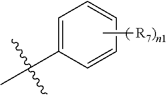

is

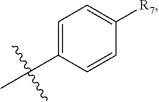

and R₇ is hydrogen, an alkyl, —O-alkyl, hydroxyl, or —NR′₁R′₂, wherein R′₁ and R′₂ are independently hydrogen or an alkyl, optionally wherein R₇ is hydrogen, an alkyl (e.g., methyl, ethyl, n-propyl, isopropyl, n-butyl, isobutyl, tert-butyl, cyclopentyl, or cyclohexyl), or —O-alkyl (e.g., methoxy or ethoxy).
Paragraph 6. The compound of any one of paragraphs 1-5, wherein R₂-R₄ are hydrogen.
Paragraph 7. The compound of any one of paragraphs 1-6, wherein R₁₂ is an alkyl (e.g., methyl, ethyl, n-propyl, isopropyl, n-butyl, isobutyl, tert-butyl, cyclopentyl, or cyclohexyl, such as methyl, isopropyl, isobutyl, or cyclohexyl).
Paragraph 8. The compound of any one of paragraphs 1-7, having any one of the structures of:

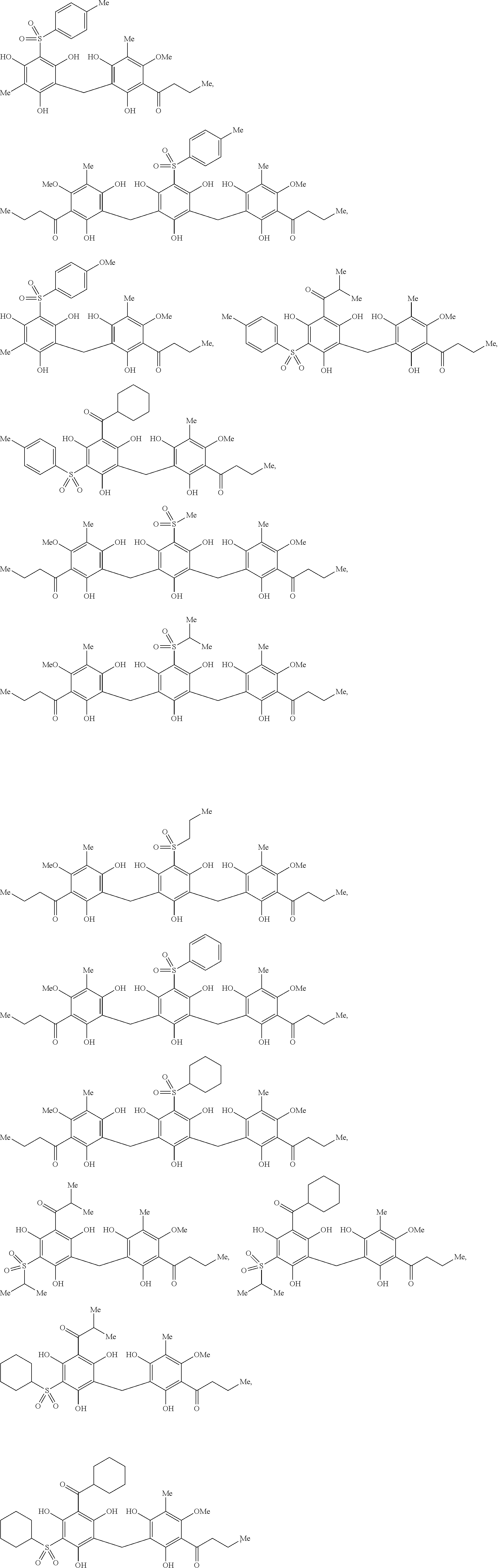

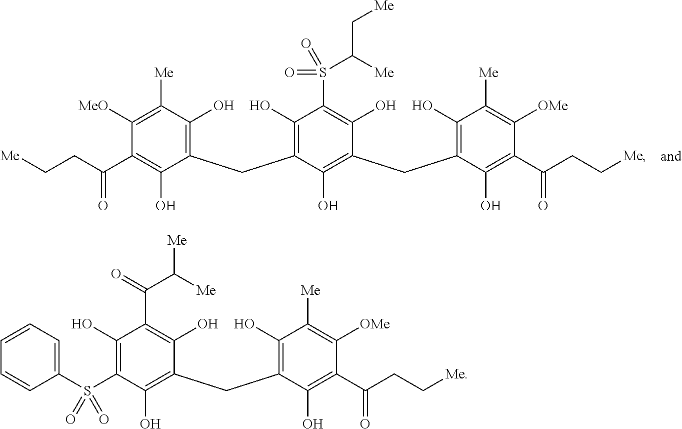

Paragraph 9. A pharmaceutical composition comprising one or more the compound(s) of any one of paragraphs 1-8, and a pharmaceutically acceptable carrier and/or excipient.
Paragraph 10. The pharmaceutical composition of paragraph 9, wherein the one or more compounds are in an effective amount to ameliorate one or more symptoms associated with a cancer in a subject.
Paragraph 11. The pharmaceutical composition of paragraph 9 or 10, further comprising a second active agent that is different from the compound(s), optionally wherein the second active agent is an anticancer agent.
Paragraph 12. A method for synthesizing the compound of any one of paragraphs 1-8, comprising:
   (1) converting a starting reagent to a sulfone intermediate or a ketone/sulfone intermediate; and
   (2) converting the sulfone intermediate or ketone/sulfone intermediate to the compound,
   wherein the starting reagent has the structure of:

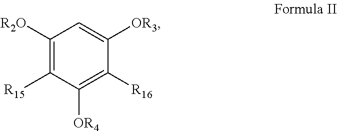

wherein the sulfone intermediate has the structure of:

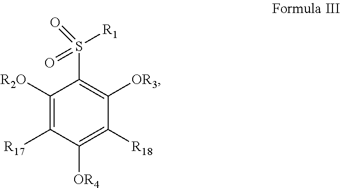

wherein the ketone/sulfone intermediate has the structure of:

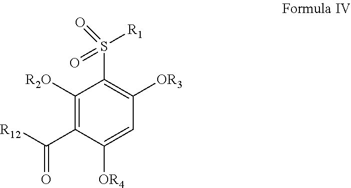

wherein:
   (i) R₁ is an alkyl or

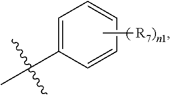

wherein n1 is an integer from 1 to 5, and R₇ is hydrogen, an alkyl, —O-alkyl, hydroxyl, or —NR′₁R′₂, wherein R′₁ and R′₂ are independently hydrogen or an alkyl;
   (ii) R₂, R₃, and R₄ are independently hydrogen or an alkyl;
   (iii) R₁₅ and R₁₆ are independently hydrogen or an alkyl, wherein at least one of R₁₅ and R₁₆ is hydrogen;
   (iv) R₁₇ and R₁₈ are independently hydrogen, an alkyl, or

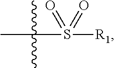

wherein at least one of R₁₇ and R₁₈ is hydrogen; and
   (v) R₁₂ is hydrogen, hydroxyl, an alkyl, or —O-alkyl.
Paragraph 13. The method of paragraph 12, wherein when R₁ is

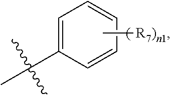

n1 is an integer from 1 to 5, R₇ is hydrogen, an alkyl, —O-alkyl, hydroxyl, or —NR′₁R′₂, and R′₁ and R′₂ are independently hydrogen or an alkyl, step (1) comprises: (a) heating a first reaction mixture at a suitable temperature for a suitable period of time to form the sulfone intermediate, wherein the first reaction mixture comprises the starting reagent and a sulfonyl halide having the structure of:

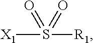

wherein X₁ is a halide (e.g., chloride, bromide, iodide, or fluoride).
Paragraph 14. The method of paragraph 12, wherein when R₁ is an alkyl, step (1) comprises:
   (a) converting the starting reagent to a lithium sulfinate intermediate; and
   (b) converting the lithium sulfinate intermediate to the sulfone intermediate,
   wherein the lithium sulfinate intermediate has the structure of:

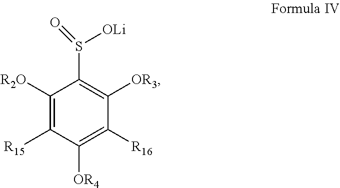

and
   wherein:
   (i) R₂, R₃, and R₄ are independently hydrogen or an alkyl;
   (ii) R₁₅ and R₁₆ are independently hydrogen or an alkyl, wherein at least one of R₁₅ and R₁₆ is hydrogen.
Paragraph 15. The method of paragraph 14, wherein step (a) comprises mixing the starting reagent, an organolithium reagent, and a sulfur dioxide surrogate reagent to form the lithium sulfinate intermediate.
Paragraph 16. The method of paragraph 15, wherein the organolithium reagent is an alkyl-lithium (e.g., n-butyllithium), and/or the sulfur dioxide surrogate has the structure of:

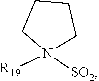

R₁₉ is an alkyl (e.g., methyl).
Paragraph 17. The method of any one of paragraphs 14-16, wherein step (b) comprises mixing the lithium sulfinate intermediate with an alkyl electrophile to form the sulfone intermediate, optionally wherein the alkyl electrophile is alkyl-X₂, X₂ is a halide (e.g., e.g., chloride, bromide, iodide, or fluoride).
Paragraph 18. The method of any one of paragraphs 13-17, wherein step (1) further comprises (c) heating a second reaction mixture at a suitable temperature for a suitable period of time to form the ketone/sulfone intermediate, wherein the second reaction mixture comprises the sulfone intermediate and a ketone reagent, and optionally wherein the ketone reagent is R₁₂—C(O)—X₃, R₁₂ is hydrogen, hydroxyl, an alkyl, or —O-alkyl, and X₃ is a halide (e.g., e.g., chloride, bromide, iodide, or fluoride).
Paragraph 19. The method of any one of paragraphs 12-18, wherein step (2) comprises (d) heating a third reaction mixture at a suitable temperature for a suitable period of time to form the compound, wherein the third reaction mixture comprises the sulfone intermediate or ketone/sulfone intermediate, and an aryl reagent having the structure of

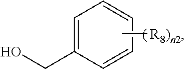

n2 is an integer from 1 to 5, R₈ is hydrogen, hydroxyl, an alkyl, —O-alkyl, or —C(O)—R₉, and R₉ is hydrogen, hydroxyl, an alkyl, or —O-alkyl.
Paragraph 20. A method for treating a cancer in a subject comprising:
   (i) administering to the subject the pharmaceutical formulation of any one of paragraphs 9-11, wherein step (i) occurs one or more times.
Paragraph 21. The method of paragraph 20, wherein the method comprises only a single administration of the pharmaceutical formulation, wherein following the administration step, an effective amount of the compound(s) to ameliorate one or more symptoms associated with the cancer in the subject is administered to the subject, or
   wherein the method comprises more than one step of administering to the subject the pharmaceutical formulation, wherein following all of the administration steps, an effective amount of the compound(s) to ameliorate one or more symptoms associated with the cancer in the subject is administered to the subject.
Paragraph 22. The method of paragraph 20 or 21, wherein the subject is a mammal.
Paragraph 23. The method of any one of paragraphs 20-22, wherein the pharmaceutical formulation is administered by oral administration, parenteral administration, mucosal administration, or topical administration, or a combination thereof.
Paragraph 24. The method of any one of paragraphs 20-23, wherein the cancer is large cell lung cancer.
Paragraph 25. The method of any one of paragraphs 20-24, further comprising administering to the subject a third active agent that is different from the compound(s), prior to, during, and/or subsequent to step (i), optionally wherein the third active agent is an anticancer agent.
Paragraph 26. A sulfone intermediate having the structure of:

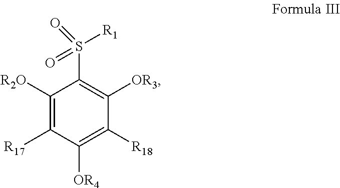

wherein:
   (i) R₁ is an alkyl (e.g., methyl, ethyl, n-propyl, isopropyl, n-butyl, isobutyl, tert-butyl, cyclopentyl, cyclohexyl, etc., for example, methyl, n-propyl, isopropyl, isobutyl, or cyclohexyl) or

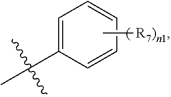

wherein n1 is an integer from 1 to 5, and R₇ is hydrogen, an alkyl, —O-alkyl, hydroxyl, or —NR′₁R′₂, wherein R′₁ and R′₂ are independently hydrogen or an alkyl;
   (ii) R₂, R₃, and R₄ are independently hydrogen or an alkyl; and
   (iii) R₁₇ and R₁₈ are independently hydrogen, an alkyl, or

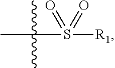

wherein at least one of R₁₇ and R₁₈ is hydrogen.
Paragraph 27. A ketone/sulfone intermediate having the structure of:

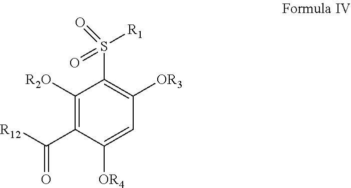

wherein:
   (i) R₁ is an alkyl (e.g., methyl, ethyl, n-propyl, isopropyl, n-butyl, isobutyl, tert-butyl, cyclopentyl, cyclohexyl, etc., for example, methyl, n-propyl, isopropyl, isobutyl, or cyclohexyl) or

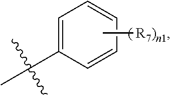

wherein n1 is an integer from 1 to 5, and R₇ is hydrogen, an alkyl, —O-alkyl, hydroxyl, or —NR′₁R′₂, wherein R′₁ and R′₂ are independently hydrogen or an alkyl;
   (ii) R₂, R₃, and R₄ are independently hydrogen or an alkyl; and
   (iii) R₁₂ is hydrogen, hydroxyl, an alkyl, or —O-alkyl.
Paragraph 28. The intermediate of paragraphs 26 or 27, the

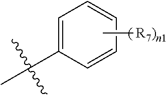

is

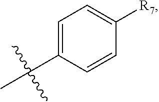

and R₇ is hydrogen, an alkyl, —O-alkyl, hydroxyl, or —NR′₁R′₂, wherein R′₁ and R′₂ are independently hydrogen or an alkyl, optionally wherein R₇ is hydrogen, an alkyl (e.g., methyl, ethyl, n-propyl, isopropyl, n-butyl, isobutyl, tert-butyl, cyclopentyl, or cyclohexyl), or —O-alkyl (e.g., methoxy or ethoxy).
Paragraph 29. The intermediate of any one of paragraphs 26-28, wherein R₂-R₄ are hydrogen.
Paragraph 30. The intermediate of any one of paragraphs 27-29, wherein R₁₂ is an alkyl (e.g., methyl, ethyl, n-propyl, isopropyl, n-butyl, isobutyl, tert-butyl, cyclopentyl, or cyclohexyl, such as methyl, isopropyl, isobutyl, or cyclohexyl).
Paragraph 31. The intermediate of any one of paragraphs 26-30, having any one of the structures of:

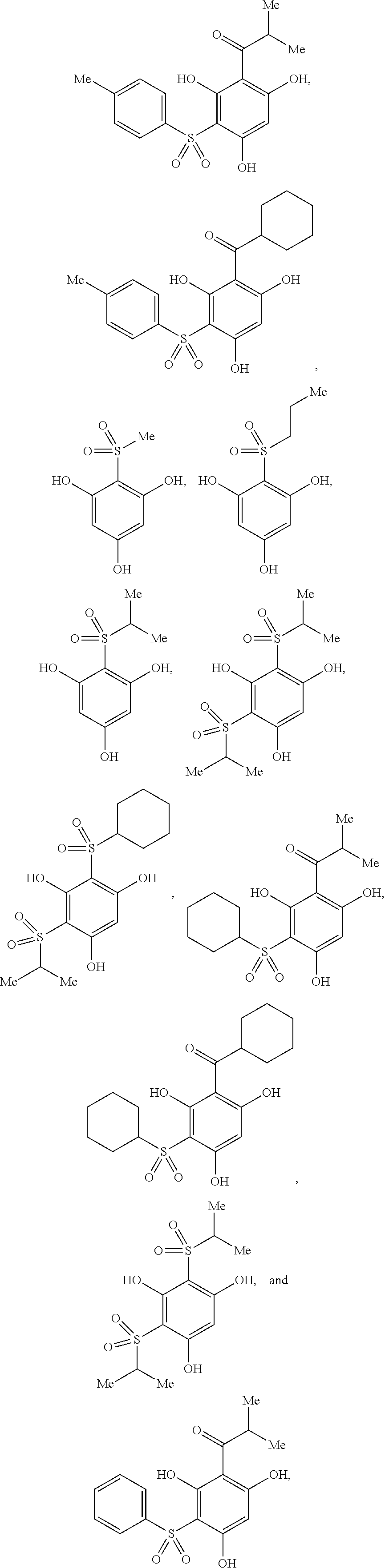

The present invention will be further understood by reference to the following non-limiting examples.

### EXAMPLES

### Example 1. Agrimol B Derivatives Show Anticancer Activity

### Materials and Methods

All reactions were carried out with continuous magnetic stirring. Air-sensitive reactions were performed under a nitrogen atmosphere using standard Schlenk techniques in oven-dried glassware. External temperature probes were used to record all reaction temperatures. All reagents with a purity >95% were obtained from commercial sources and used without further purification. Flash column chromatography was carried out with silica gel 60 (0.040-0.063 mm). Analytical thin layer chromatography (TLC) was carried out using silica gel F254 glass-backed plates.

¹H and ¹³C NMR NMR spectra were recorded on a Bruker AVANCE NEO 600 MHz spectrometer using the residual solvent as an internal standard and reported as follows: chemical shift δ in ppm (multiplicity, coupling constant Jin Hz, number of protons) for ¹H NMR spectra and chemical shift δ in ppm for ¹³C NMR spectra. Multiplicities are abbreviated as follows: s=singlet, d=doublet, t=triplet, q=quartet, m=multiplet, or combinations thereof. High-resolution mass spectra (HR-MS) were recorded on an Agilent UPLC-6546QTOF mass spectrometry.

Generally, a direct Friedel-Crafts sulfonylation was developed to produce the desired A ring in a straightforward way (Scheme 1). This reaction can be applied to electron-neutral and electron-donating groups and excludes an oxidation process, addressing the shortcomings of the previous method (H. Burton and E. Hoggarth, Journal of the Chemical Society, 1945, 468-470).

### Scheme 1. Synthesis of Exemplary Aryl Sulfone A Ring

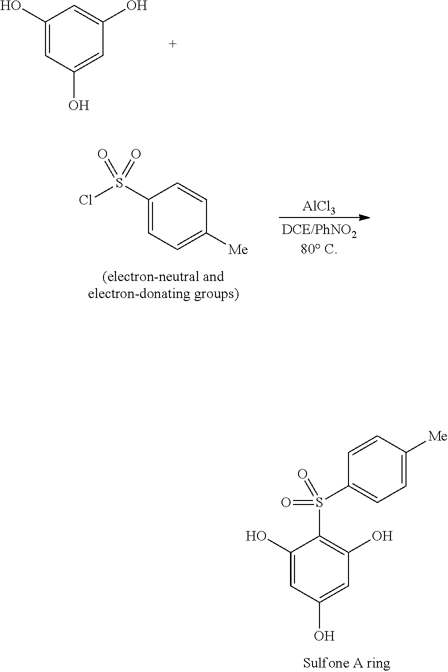 [SMILES: Oc1cc(O)cc(O)c1 ; Cc1ccc(S(=O)(=O)Cl)cc1 ; "AlCl3" ; Cc1ccc(S(=O)(=O)c2c(O)cc(O)cc2O)cc1]

Another approach was also developed to include alkyl sulfone into the A ring (Scheme 2). Specifically, a SO₂ surrogate was used to introduce the sulfonyl (—SO₂—) group, producing lithium sulfinate (RSO₂Li) as a versatile intermediate. The sulfinate was then reacted with various alkyl electrophiles. After deprotection of methoxy groups with BBr₃, the desired sulfone-incorporated A ring was obtained.

### Scheme 2. Synthesis of Exemplary Alkyl Sulfone A Ring

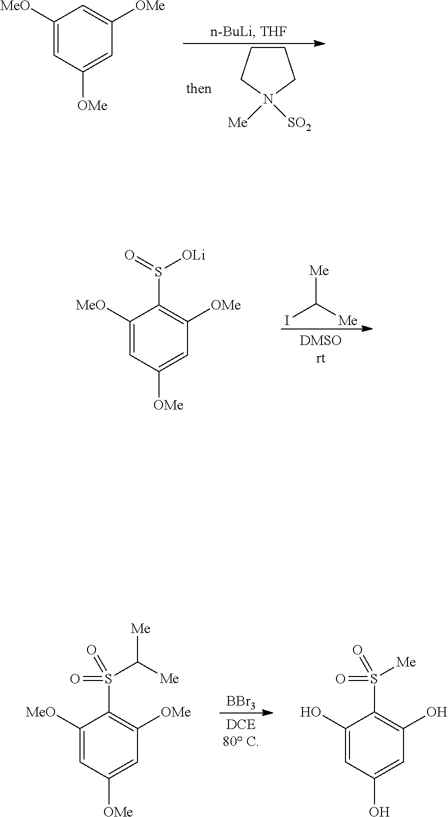

A series of ketone/sulfone-incorporated A rings were also prepared using BF₃·Et₂O as the Lewis acid (Scheme 3).

### Scheme 3. Synthesis of Exemplary Ketone/Sulfone A Ring

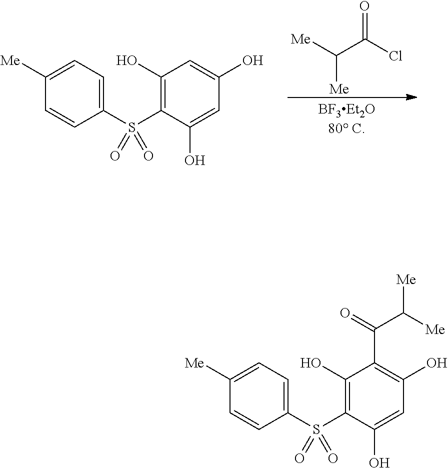 [SMILES: Cc1ccc(S(=O)(=O)c2c(O)cc(O)cc2O)cc1 ; CC(C)C(=O)Cl ; Cc1ccc(S(=O)(=O)c2c(O)cc(O)c(C(=O)C(C)C)c2O)cc1]

After coupling the sulfone-incorporated A rings with the B rings, a series of AGB derivatives incorporating sulfone or ketone/sulfone groups were prepared (Scheme 4).

### Scheme 4. Synthesis of Exemplary AGB Derivatives.

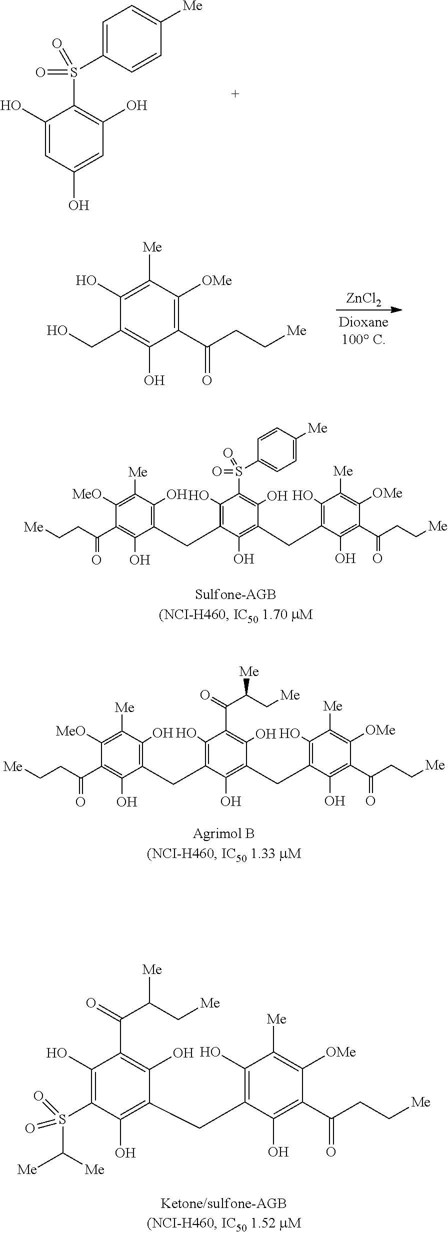 [SMILES: Cc1ccc(S(=O)(=O)c2c(O)cc(O)cc2O)cc1 ; CCCC(=O)c1c(O)c(CO)c(O)c(C)c1OC ; "ZnCl2" ; CCCC(=O)c1c(O)c(Cc2c(O)c(Cc3c(O)c(C)c(OC)c(C(=O)CCC)c3O)c(O)c(S(=O)(=O)c3ccc(C)cc3)c2O)c(O)c(C)c1OC ; CCCC(=O)c1c(O)c(Cc2c(O)c(Cc3c(O)c(C)c(OC)c(C(=O)CCC)c3O)c(O)c(C(=O)[C@@H](C)CC)c2O)c(O)c(C)c1OC ; CCCC(=O)c1c(O)c(Cc2c(O)c(C(=O)C(C)CC)c(O)c(S(=O)(=O)C(C)C)c2O)c(O)c(C)c1OC]

The anticancer activity of exemplary AGB derivatives were examined using NCI-H460 large lung cancer cells.

### A Ring Synthesis with Aryl Sulfonyl Chloride (Method A)

### 1,3,5-Trimethoxy-2-(phenylsulfonyl)benzene

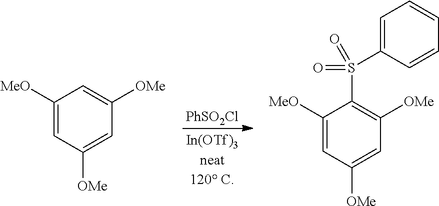 [SMILES: "PhSO2Cl" ; COc1cc(OC)cc(OC)c1 ; COc1cc(OC)c(S(=O)(=O)c2ccccc2)c(OC)c1]

A solution of 1,3,5-trimethoxybenzene (1008 mg, 6.00 mmol, 2.0 equiv.), benzenesulfonyl chloride (0.38 mL, 3.00 mmol, 1.0 equiv.) and In(OTf)₃ (85 mg, 0.15 mmol, 5 mol %) was stirred neat at 120° C. for 30 min. The reaction was quenched with H₂O and extracted with EtOAc (×2). The combined organic phases were dried (Na₂SO₄), filtered, and concentrated. Purification by flash column chromatography (0-20-40% EtOAc in n-hexane) afforded the compound (185 mg, 20%). ¹H NMR (600 MHz, CDCl₃) δ 7.93 (d, J=7.5 Hz, 2H), 7.49 (t, J=7.5 Hz, 1H), 7.43 (t, J=7.5 Hz, 2H), 6.05 (s, 2H), 3.80 (s, 3H), 3.74 (s, 6H). ¹³C NMR (151 MHz, CDCl₃) δ 165.3, 161.1, 144.9, 132.2, 128.3, 127.1, 110.7, 91.6, 56.5, 55.6. HR-MS (positive mode): calculated for [M+H]⁺ 309.0792; Found 309.0794.

### 2-Tosylbenzene-1,3,5-triol

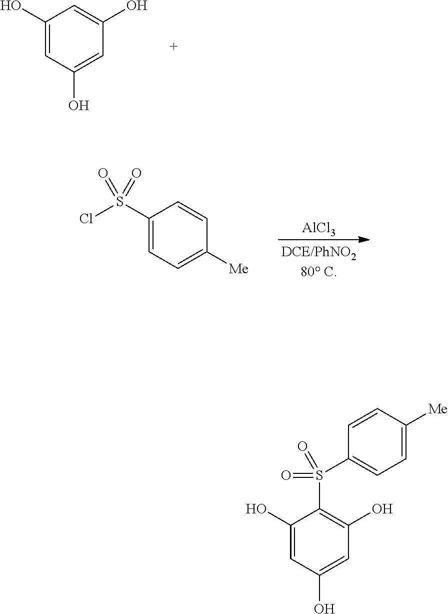 [SMILES: Oc1cc(O)cc(O)c1 ; Cc1ccc(S(=O)(=O)Cl)cc1 ; "AlCl3" ; Cc1ccc(S(=O)(=O)c2c(O)cc(O)cc2O)cc1]

A solution of phloroglucinol (189 mg, 1.50 mmol, 1.0 equiv.), anhydrous aluminium chloride (AlCl₃, 299 mg, 2.25 mmol, 1.5 equiv.), and 4-toluenesulfonyl chloride (430 mg, 2.25 mmol, 1.5 equiv.) in 1,2-dichloroethane and nitrobenzene (1:1, 1.0 mL) was heated at 80° C. for 3 h. After cooling to room temperature (“rt”), the mixture was quenched with 1M HCl solution and extracted with ethyl acetate (×2). The combined organic phases were dried (Na₂SO₄), filtered, and concentrated. Purification by flash column chromatography (100% CH₂Cl₂ then 0-50% EtOAc in n-hexane) afforded the compound (170 mg, 40%). ¹H NMR (600 MHz, MeOD) δ 7.80 (d, J=8.0 Hz, 2H), 7.35 (d, J=8.0 Hz, 2H), 5.82 (s, 2H), 2.42 (s, 3H). ¹³C NMR (151 MHz, MeOD) δ 166.0, 160.4, 145.4, 141.3, 130.2, 128.5, 103.5, 96.4, 21.5. HR-MS (negative mode): calculated for [M−H]⁻ 279.0332; Found 279.0332.

### 2-Methyl-4-tosylbenzene-1,3,5-triol

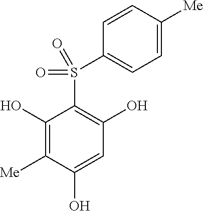 [SMILES: Cc1ccc(S(=O)(=O)c2c(O)cc(O)c(C)c2O)cc1]

Following method A using 2-methylbenzene-1,3,5-triol (210 mg, 1.50 mmol, 1.0 equiv.), AlCl₃ (299 mg, 2.25 mmol, 1.5 equiv.), 4-toluenesulfonyl chloride (430 mg, 2.25 mmol, 1.5 equiv.), and 1,2-dichloroethane and nitrobenzene (0.5 mL and 0.5 mL). Purification by flash column chromatography (100% CH₂Cl₂ then 0-40% EtOAc in n-hexane) afforded the compound (152 mg, 34%). ¹H NMR (600 MHz, DMSO) δ 10.40 (s, 1H), 10.29 (s, 1H), 10.23 (s, 1H), 7.76 (d, J=8.5 Hz, 2H), 7.37 (d, J=8.5 Hz, 2H), 5.93 (s, 1H), 2.37 (s, 3H), 1.86 (s, 3H). ¹³C NMR (151 MHz, DMSO) δ 162.0, 155.9, 155.8, 143.6, 139.8, 129.3, 127.1, 102.3, 101.7, 94.7, 21.0, 7.7. HR-MS (negative mode): calculated for [M−H]⁻ 293.0489; Found 293.0486.

### 2-((4-Methoxyphenyl)sulfonyl)-4-methylbenzene-1,3,5-triol

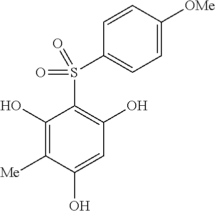 [SMILES: COc1ccc(S(=O)(=O)c2c(O)cc(O)c(C)c2O)cc1]

Following method A using 2-methylbenzene-1,3,5-triol (210 mg, 1.50 mmol, 1.0 equiv.), AlCl₃ (299 mg, 2.25 mmol, 1.5 equiv.), 4-methoxybenzenesulfonyl chloride (466 mg, 2.25 mmol, 1.5 equiv.), and 1,2-dichloroethane and nitrobenzene (0.5 mL and 0.5 mL). Purification by flash column chromatography (50-100% CH₂Cl₂ in n-hexane then 0-20-40% EtOAc in n-hexane) afforded the compound (170 mg, 37%). ¹H NMR (600 MHz, MeOD) δ 7.85 (d, J=9.0 Hz, 2H), 7.02 (d, J=9.0 Hz, 2H), 5.88 (s, 1H), 3.85 (s, 3H), 1.94 (s, 3H). ¹³C NMR (151 MHz, CDCl₃) δ 164.9, 163.6, 157.4, 157.1, 135.8, 130.6, 114.8, 105.3, 104.8, 103.8, 95.7, 7.7. HR-MS (negative mode): calculated for [M−H]⁻ 309.0438; Found 309.0440.

### 2,4-Bis((4-methoxyphenyl)sulfonyl)-6-methylbenzene-1,3,5-triol

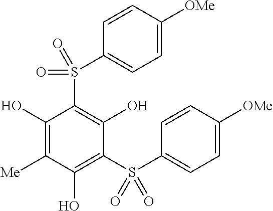 [SMILES: COc1ccc(S(=O)(=O)c2c(O)c(C)c(O)c(S(=O)(=O)c3ccc(OC)cc3)c2O)cc1]

Following method A using 2-methylbenzene-1,3,5-triol (210 mg, 1.50 mmol, 1.0 equiv.), AlCl₃ (399 mg, 3.00 mmol, 2.0 equiv.), 4-methoxybenzenesulfonyl chloride (621 mg, 3.00 mmol, 2.0 equiv.), and 1,2-dichloroethane and nitrobenzene (1.5 mL and 1.5 mL) Purification by flash column chromatography (100% CH₂Cl₂) afforded the compound (188 mg, 26%). ¹H NMR (600 MHz, CDCl₃) δ 10.26 (s, 2H), 9.71 (s, 1H), 7.84 (d, J=9.0 Hz, 4H), 6.97 (d, J=9.0 Hz, 4H), 3.88 (s, 6H), 2.01 (s, 3H). ¹³C NMR (151 MHz, CDCl₃) δ 164.2, 159.9, 154.8, 132.7, 129.4, 114.6, 107.6, 104.1, 55.9, 7.8. HR-MS (negative mode): calculated for [M−H]⁻ 479.0475; Found 479.0478.

### Lithium 2,4,6-trimethoxybenzenesulfinate

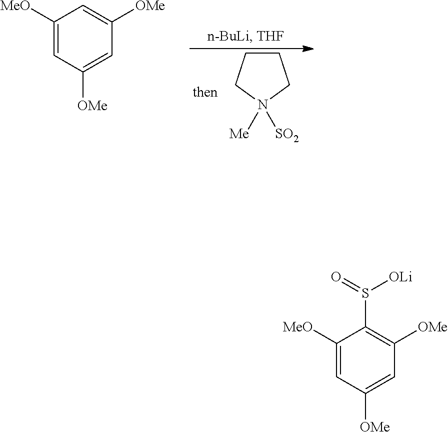

n-Butyllithium (9.67 mL, 2.07 M in THF, 20.0 mmol, 1.11 equiv) was added slowly to a solution of 1,3,5-trimethoxybenzene (3.36 g, 20 mmol, 1.11 equiv.) in THF (50 mL) at rt. The reaction was stirred at 40° C. for 3 h. After cooling to rt, sulfur dioxide 1-methylpyrrolidine adduct (2.25 mL, density: 1.19 g/mL, 18.0 mmol, 1.00 equiv.) was added dropwise and solid was formed. After stirring at rt for 30 min, the solid was filtered under vacuum, washed with Et₂O, and further dried under suction to afford the compound (4.30 g, 99%). ¹H NMR (600 MHz, MeOD) δ 6.14 (s, 2H), 3.79 (m, 9H). ¹³C NMR (151 MHz, MeOD) δ 163.8, 161.6, 161.0, 92.0, 56.1, 55.8. HR-MS (positive mode): calculated for [M+H]⁺ 239.0560; Found 239.0557.

### General Procedure for A Ring Synthesis with Arylsulfinate (Method B)

### 2-(Methylsulfonyl)benzene-1,3,5-triol

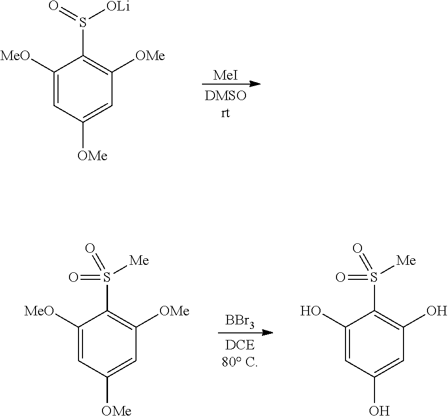

Alkylation: A mixture of lithium 2,4,6-trimethoxybenzenesulfinate (240 mg, 1.0 mmol, 1.0 equiv.) and methyl iodide (1.5 mL, 2M solution in tert-Butyl methyl ether, 3.0 mmol, 3.0 equiv.) in DMSO (5.0 mL) was stirred at rt for overnight. The reaction was quenched with H₂O/brine and extracted with EtOAc (×2). The combined organic phases were dried (Na₂SO₄), filtered and concentrated. Purification by flash column chromatography (0-40-50% EtOAc in n-hexane) afforded the trimethoxybenzene sulfone compound (70 mg, 28%). ¹H NMR (600 MHz, CDCl₃) δ 6.12 (s, 2H), 3.88 (s, 6H), 3.84 (s, 3H), 3.21 (s, 3H). ¹³C NMR (151 MHz, CDCl₃) δ 165.0, 160.9, 110.7, 91.6, 56.7, 55.7, 45.9. HR-MS (positive mode): calculated for [M+H]⁺ 247.0635; Found 247.0635.

Demethylation: BBr₃ (0.37 mL, 3.8 mmol, 12 equiv.) was added dropwise to a solution of the trimethoxybenzene sulfone compound (81 mg, 0.33 mmol, 1.0 equiv.) in DCE (3.3 mL) at rt. The reaction was stirred at 80° C. for 1 h. After cooling to rt, the reaction was quenched by slowly transferring it to ice-cold H₂O and extracted with EtOAc (×2). The combined organic phases were dried (Na₂SO₄), filtered, and concentrated. Purification by flash column chromatography (0-50-60% EtOAc in n-hexane) afforded the compound (46 mg, 68%). ¹H NMR (600 MHz, CD₃CN) δ 5.94 (s, 2H), 3.25 (s, 3H). ¹³C NMR (151 MHz, CD₃CN) δ 164.5, 159.5, 103.6, 96.6, 45.1. HR-MS (negative mode): calculated for [M−H]⁻ 203.0019; Found 203.0020.

### 2-(Propylsulfonyl)benzene-1,3,5-triol

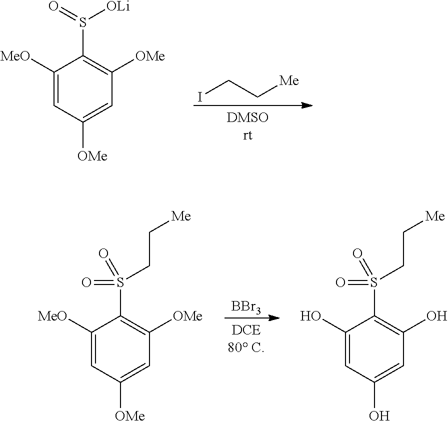

Alkylation: Following method B using lithium 2,4,6-trimethoxybenzenesulfinate (238 mg, 1.0 mmol, 1.0 equiv.), n-propyl iodide (0.29 mL, 3.0 mmol, 3.0 equiv.) in DMSO (5.0 mL). Purification by flash column chromatography (0-50-60% EtOAc in n-hexane) afforded the trimethoxybenzene sulfone compound (90 mg, 33%). ¹H NMR (600 MHz, CDCl₃) δ 6.13 (s, 2H), 3.89 (s, 6H), 3.85 (s, 3H), 3.30-3.25 (m, 2H), 1.84-1.75 (m, 2H), 1.00 (t, J=7.5 Hz, 3H). ¹³C NMR (151 MHz, CDCl₃) δ 165.0, 161.6, 109.2, 91.8, 58.8, 56.8, 55.7, 16.4, 13.2. HR-MS (positive mode): calculated for [M+H]+ 275.0948; Found 275.0951.

Demethylation: Following method B using the trimethoxybenzene sulfone compound (90 mg, 0.33 mmol, 1.0 equiv.) and BBr₃ (0.37 mL, 3.8 mmol, 12 equiv.) in DCE (3.3 mL). Purification by flash column chromatography (0-40% EtOAc in n-hexane) afforded the compound (52 mg, 68%). ¹H NMR (600 MHz, MeOD) δ 5.88 (s, 2H), 3.44-3.35 (m, 2H), 1.77-1.64 (m, 2H), 1.01 (t, J=7.5 Hz, 3H). ¹³C NMR (151 MHz, MeOD) δ 165.9, 160.8, 100.9, 96.4, 58.3, 17.6, 13.0. HR-MS (negative mode): calculated for [M−H]⁻ 231.0332; Found 231.0337.

### 2-(Isopropylsulfonyl)benzene-1,3,5-triol

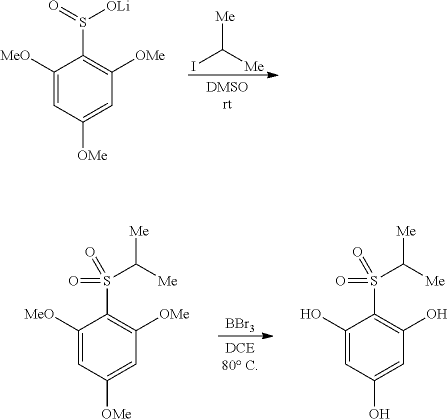

Alkylation: Following method B using lithium 2,4,6-trimethoxybenzenesulfinate (480 mg, 2.0 mmol, 1.0 equiv.) and isopropyl iodide (0.60 mL, 6.0 mmol, 3.0 equiv.) in DMSO (10 mL). Purification by flash column chromatography (0-50% EtOAc in n-hexane) afforded the trimethoxybenzene sulfone compound (101 mg, 18%). ¹H NMR (600 MHz, CDCl₃) δ 6.13 (s, 2H), 3.88 (s, 6H), 3.85 (s, 3H), 3.60 (hept, J=7.0 Hz, 1H), 1.31 (d, J=7.0 Hz, 6H). ¹³C NMR (151 MHz, CDCl₃) δ 164.9, 161.9, 107.5, 91.8, 56.7, 55.5, 55.0, 15.1. HR-MS (positive mode): calculated for [M+H]⁺ 275.0948; Found 275.0951.

Demethylation: Following method B using the trimethoxybenzene sulfone compound (55 mg, 0.20 mmol, 1.0 equiv.) and BBr₃ (0.44 mL, 4.6 mmol, 23 equiv.) in DCE (2.0 mL). Purification by flash column chromatography (0-25-40% EtOAc in n-hexane) afforded the compound (30 mg, 65%). ¹H NMR (600 MHz, MeOD) δ 5.88 (s, 2H), 3.77 (hept, J=7.0 Hz, 1H), 1.29 (d, J=7.0 Hz, 6H). ¹³C NMR (151 MHz, MeOD) δ 166.0, 161.1, 99.2, 96.5, 55.7, 15.0. HR-MS (negative mode): calculated for [M−H]⁻ 231.0332; Found 231.0330.

### 2-(sec-Butylsulfonyl)benzene-1,3,5-triol

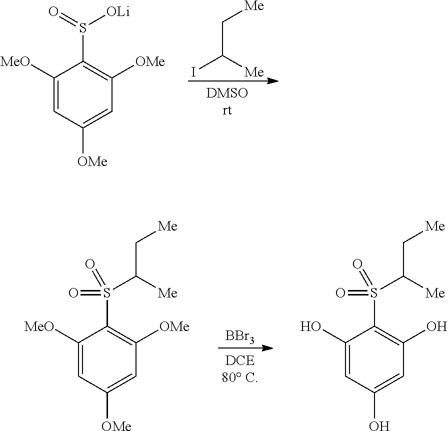

Alkylation: Following method B using lithium 2,4,6-trimethoxybenzenesulfinate (238 mg, 1.0 mmol, 1.0 equiv.) and sec-butyl iodide (0.35 mL, 3.0 mmol, 3.0 equiv.) in DMSO (5.0 mL) for 3 h. Purification by flash column chromatography (0-40% EtOAc in n-hexane) afforded the trimethoxybenzene sulfone compound (116 mg, 40%). ¹H NMR (600 MHz, CD₃CN) δ 6.25 (s, 2H), 3.85 (s, 3H), 3.82 (s, 6H), 3.39-3.32 (m, 1H), 1.86-1.80 (m, 1H), 1.48-1.40 (m, 1H), 1.19 (d, J=7.0 Hz, 3H), 0.94 (t, J=7.5 Hz, 3H). ¹³C NMR (151 MHz, CD₃CN) δ 166.1, 162.6, 108.5, 92.9, 61.4, 57.3, 56.5, 23.1, 12.4, 11.5. HR-MS (positive mode): calculated for [M+H]⁺ 289.1105; Found 289.1107.

Demethylation: Following method B using the trimethoxybenzene sulfone compound (116 mg, 0.4 mmol, 1.0 equiv.) and BBr₃ (0.89 mL, 9.2 mmol, 23 equiv.) in DCE (4.0 mL) for 2 h. Purification by flash column chromatography (0-35% EtOAc in n-hexane) afforded the compound (38 mg, 39%). ¹H NMR (600 MHz, CD₃CN) δ 5.95 (s, 2H), 3.39 (dqd, J=9.5, 7.0, 4.0 Hz, 1H), 1.94-1.85 (m, 1H), 1.56-1.46 (m, 1H), 1.26 (d, J=7.0 Hz, 3H), 0.97 (t, J=7.5 Hz, 3H). ¹³C NMR (151 MHz, CD₃CN) δ 164.9, 160.3, 99.7, 96.9, 62.1, 22.7, 12.1, 11.2. HR-MS (negative mode): calculated for [M−H]⁻ 245.0489; Found 245.0492.

### 2-(Cyclohexylsulfonyl)benzene-1,3,5-triol

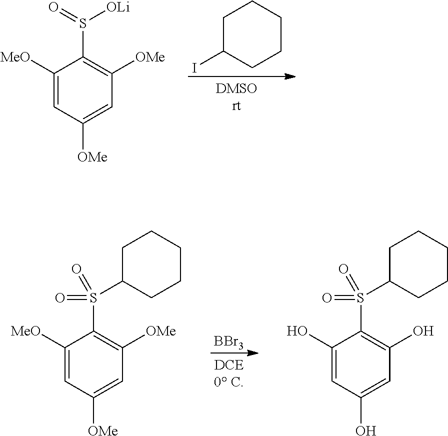

Alkylation: Following method B using lithium 2,4,6-trimethoxybenzenesulfinate (1904 mg, 8.0 mmol, 1.0 equiv.) and cyclohexyl iodide (5.2 mL, 40 mmol, 5.0 equiv.) in DMSO (80 mL). Purification by flash column chromatography (0-40% EtOAc in n-hexane) afforded the trimethoxybenzene sulfone compound (998 mg, 40%). ¹H NMR (600 MHz, MeOD) δ 6.31 (s, 2H), 3.89 (s, 3H), 3.86 (s, 6H), 3.35-3.29 (m, 1H), 1.93 (dd, J=13.5, 3.5 Hz, 2H), 1.87 (dt, J=13.0, 3.5 Hz, 2H), 1.70 (dt, J=12.5, 3.5 Hz, 1H), 1.52 (app. qd, J=12.5, 3.5 Hz, 2H), 1.33-1.16 (m, 3H). ¹³C NMR (151 MHz, MeOD) δ 167.4, 163.3, 106.9, 92.9, 64.3, 57.1, 56.3, 26.5, 26.2, 26.0. HR-MS (positive mode): calculated for [M+H]⁺ 315.1261; Found 315.1265.

Demethylation: Following method B using the trimethoxybenzene sulfone compound (140 mg, 0.45 mmol, 1.0 equiv.) and BBr₃ (1.00 mL, 10.0 mmol, 23 equiv.) in DCE (4.5 mL) at 0° C. for 2 h. Purification by flash column chromatography (0-30-40% EtOAc in n-hexane) afforded the compound (60 mg, 49%). ¹H NMR (600 MHz, MeOD) δ 5.88 (s, 2H), 3.49 (tt, J=12.0, 3.5 Hz, 1H), 2.00-1.94 (m, 2H), 1.90 (tt, J=13.5, 3.5 Hz, 2H), 1.71 (tt, J=14.5, 3.0 Hz, 1H), 1.57-1.48 (m, 2H), 1.36-1.26 (m, 2H), 1.23 (tt, J=13.0, 3.0 Hz, 1H). ¹³C NMR (151 MHz, MeOD) δ 166.0, 161.2, 99.1, 96.5, 63.6, 26.4, 26.2, 25.8. HR-MS (negative mode): calculated for [M−H]⁻ 271.0645; Found 271.0647.

### General Procedure for Sulfone-Ketone A Ring Synthesis (Method C)

### 2-Methyl-1-(2,4,6-trihydroxy-3-tosylphenyl)propan-1-one

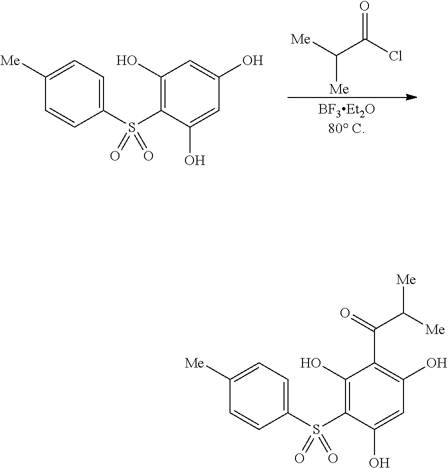 [SMILES: Cc1ccc(S(=O)(=O)c2c(O)cc(O)cc2O)cc1 ; CC(C)C(=O)Cl ; Cc1ccc(S(=O)(=O)c2c(O)cc(O)c(C(=O)C(C)C)c2O)cc1]

A solution of sulfone (280 mg, 1.0 mmol, 1.0 equiv.), isobutyryl chloride (0.12 mL, 1.1 mmol, 1.1 equiv.) and BF₃·Et₂O (1.5 mL) was stirred at 80° C. for 3 h. The reaction was slowly quenched by adding to ˜20 mL of 20% aq. KOAc solution at 0° C. After stirring at rt for 2 h, the mixture was slowly neutralized by sat. aq. NaHCO₃, followed by extraction with EtOAc (×2). The combined organic phases were dried (Na₂SO₄), filtered and concentrated. Purification by flash column chromatography (0-30% EtOAc in n-hexane) afforded the compound (228 mg, 65%). ¹H NMR (600 MHz, CDCl₃) δ 13.37 (br. s, 1H), 10.93 (br. s, 1H), 9.43 (br. s, 1H), 7.79 (d, J=8.5 Hz, 2H), 7.35 (d, J=8.5 Hz, 2H), 6.04 (s, 1H), 3.80 (hept, J=6.5 Hz, 1H), 2.44 (s, 3H), 1.16 (d, J=6.5 Hz, 6H). ¹³C NMR (151 MHz, CDCl₃) δ 211.1, 170.6, 161.7, 161.4, 145.8, 138.6, 130.4, 126.2, 104.8, 101.5, 98.8, 40.1, 21.8, 19.2. HR-MS (positive mode): calculated for [M+H]⁺ 351.0897; Found 351.0895.

### 2-Methyl-1-(2,4,6-trihydroxy-3-(phenylsulfonyl)phenyl)propan-1-one

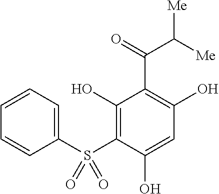 [SMILES: CC(C)C(=O)c1c(O)cc(O)c(S(=O)(=O)c2ccccc2)c1O]

Following method C using sulfone (199 mg, 0.75 mmol, 1.0 equiv.), isobutyryl chloride (0.09 mL, 0.8 mmol, 1.1 equiv.), and BF₃·Et₂O (1.1 mL). Purification by flash column chromatography (0-25% EtOAc in n-hexane) afforded the compound (141 mg, 56%). ¹H NMR (600 MHz, CDCl₃) δ 13.15 (br. s, 1H), 11.16 (br. s, 1H), 9.47 (br. s, 1H), 7.92 (d, J=7.5 Hz, 2H), 7.66 (td, J=7.5, 1.0 Hz, 1H), 7.57 (t, J=7.5 Hz, 2H), 6.05 (s, 1H), 3.80 (hept, J=6.5 Hz, 1H), 1.16 (d, J=6.5 Hz, 6H). ¹³C NMR (151 MHz, CDCl₃) δ 211.1, 170.5, 162.1, 161.5, 141.5, 134.5, 129.7, 126.2, 104.8, 101.2, 98.8, 40.1, 19.2. HR-MS (positive mode): calculated for [M+H]⁺ 337.0741; Found 337.0743.

### 1-(3-(Cyclohexylsulfonyl)-2,4,6-trihydroxyphenyl)-2-methylpropan-1-one

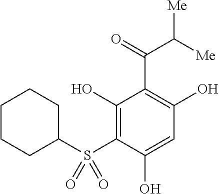 [SMILES: CC(C)C(=O)c1c(O)cc(O)c(S(=O)(=O)C2CCCCC2)c1O]

Following method C using sulfone (45 mg, 0.17 mmol, 1.0 equiv.), isobutyryl chloride (19 μL, 0.18 mmol, 1.1 equiv.), and BF₃·Et₂O (0.25 mL). Purification by flash column chromatography (0-20% EtOAc in cyclohexane) afforded the compound (31 mg, 55%). ¹H NMR (600 MHz, CDCl₃) δ 13.54 (br. s, 1H), 10.93 (br. s, 1H), 9.30 (br. s, 1H), 6.07 (s, 1H), 3.80 (hept, J=7.0 Hz, 1H), 3.11 (tt, J=12.5, 3.5 Hz, 1H), 2.11 (d, J=13.5 Hz, 2H), 1.92 (app. dt, J=13.5, 3.5 Hz, 2H), 1.72 (app. dt, J=13.5, 3.5 Hz, 1H), 1.51 (app. qd, J=12.5, 3.5 Hz, 2H), 1.33-1.23 (m, 2H), 1.23-1.15 (m, 7H). ¹³C NMR (151 MHz, CDCl₃) δ 211.2, 170.8, 162.8, 162.5, 104.6, 98.5, 96.1, 65.7, 40.0, 25.0, 24.8, 19.2, 1×AlkC not observed. HR-MS (positive mode): calculated for [M+H]⁺ 343.1210; Found 343.1212.

### 2-Methyl-1-(2,4,6-trihydroxy-3-isopropylsulfonyl)phenyl)propan-1-one

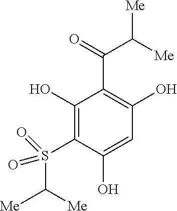 [SMILES: CC(C)C(=O)c1c(O)cc(O)c(S(=O)(=O)C(C)C)c1O]

Following method C using sulfone (35 mg, 0.15 mmol, 1.0 equiv.), isobutyryl chloride (18 μL, 0.17 mmol, 1.1 equiv.), and BF₃·Et₂O (0.23 mL). Purification by flash column chromatography (0-25% EtOAc in n-hexane) afforded the compound (25 mg, 55%). ¹H NMR (600 MHz, CD₃CN) δ 14.71 (br. s, 1H), 10.41 (app. br. s, 2H), 5.96 (s, 1H), 3.86 (hept, J=7.0 Hz, 1H), 3.80 (hept, J=7.0 Hz, 1H), 1.28 (d, J=7.0 Hz, 6H), 1.14 (d, J=7.0 Hz, 6H). ¹³C NMR (151 MHz, CD₃CN) δ 212.4, 167.8, 167.7, 164.9, 104.4, 100.0, 96.8, 55.7, 40.3, 19.3, 14.9. HR-MS (negative mode): calculated for [M−H]⁻ 301.0751; Found 301.0755.

### Cyclohexyl(2,4,6-trihydroxy-3-tosylphenyl)methanone

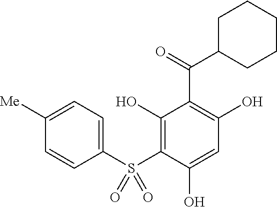 [SMILES: Cc1ccc(S(=O)(=O)c2c(O)cc(O)c(C(=O)C3CCCCC3)c2O)cc1]

Following method C using sulfone (56 mg, 0.20 mmol, 1.0 equiv.), cyclohexanecarbonyl chloride (30 μL, 0.22 mmol, 1.1 equiv.), and BF₃·Et₂O (0.30 mL). Purification by flash column chromatography (0-25% EtOAc in n-hexane) afforded the compound (52 mg, 67%). ¹H NMR (600 MHz, CDCl₃) δ 13.15 (br. s, 1H), 11.26 (br. s, 1H), 9.51 (br. s, 1H), 7.80 (d, J=8.5 Hz, 2H), 7.35 (d, J=8.5 Hz, 2H), 6.02 (s, 1H), 3.50 (tt, J=11.0, 3.0 Hz, 1H), 2.43 (s, 3H), 1.90-1.85 (m, 2H), 1.84-1.79 (m, 2H), 1.74-1.69 (m, 1H), 1.42-1.29 (m, 4H), 1.27-1.19 (m, 1H). ¹³C NMR (151 MHz, CDCl₃) δ 210.2, 170.3, 162.1, 161.4, 145.7, 138.6, 130.3, 126.3, 104.9, 101.6, 98.6, 50.3, 29.5, 26.1, 21.8, 1×AlkC not observed. HR-MS (positive mode): calculated for [M+H]⁺ 391.1210; Found 391.1209.

### Cyclohexyl(3-(cyclohexylsulfonyl)-2,4,6-trihydroxyphenyl)methanone

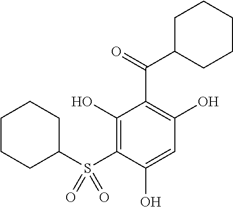 [SMILES: O=C(c1c(O)cc(O)c(S(=O)(=O)C2CCCCC2)c1O)C1CCCCC1]

Following method C using sulfone (87 mg, 0.32 mmol, 1.0 equiv.), cyclohexanecarbonyl chloride (50 μL, 0.37 mmol, 1.2 equiv.), and BF₃·Et₂O (0.49 mL). Purification by flash column chromatography (0-20% EtOAc in n-hexane) afforded the compound (67 mg, 55%). ¹H NMR (600 MHz, CDCl₃) δ 13.91 (br. s, 1H), 10.56 (br. s, 1H), 9.19 (br. s, 1H), 6.08 (s, 1H), 3.50 (tt, J=11.0, 3.0 Hz, 1H), 3.07 (tt, J=13.0, 3.5 Hz, 1H), 2.14-2.09 (m, 2H), 1.97-1.87 (m, 4H), 1.85-1.79 (m, 2H), 1.76-1.69 (m, 2H), 1.56-1.46 (m, 2H), 1.46-1.37 (m, 2H), 1.37-1.16 (m, 6H). ¹³C NMR (151 MHz, CDCl₃) δ 210.2, 171.1, 162.6, 162.4, 104.9, 98.7, 96.0, 65.8, 50.4, 31.1, 29.5, 26.2, 25.0, 24.8. HR-MS (positive mode): calculated for [M+H]⁺ 383.1523; Found 383.1524.

### Cyclohexyl(2,4,6-trihydroxy-3-(isopropylsulfonyl)phenyl)methanone

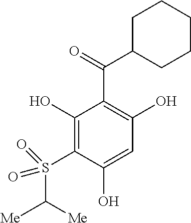 [SMILES: CC(C)S(=O)(=O)c1c(O)cc(O)c(C(=O)C2CCCCC2)c1O]

Following method C using sulfone (35 mg, 0.15 mmol, 1.0 equiv.), cyclohexanecarbonyl chloride (22 μL, 0.17 mmol, 1.1 equiv.), and BF₃·Et₂O (0.23 mL). Purification by flash column chromatography (0-30% EtOAc in n-hexane) afforded the compound (20 mg, 40%). ¹H NMR (600 MHz, CDCl₃) δ 13.54 (br. s, 1H), 10.95 (br. s, 1H), 9.31 (br. s, 1H), 6.07 (s, 1H), 3.50 (tt, J=11.5, 3.0 Hz, 1H), 3.41 (hept, J=7.0 Hz, 1H), 1.89 (d, J=15.0 Hz, 2H), 1.82 (dt, J=12.0, 3.5 Hz, 2H), 1.74-1.68 (m, 1H), 1.45-1.36 (m, 8H), 1.32 (app. qt, J=12.5, 2.5 Hz, 2H), 1.28-1.18 (m, 1H). ¹³C NMR (151 MHz, CDCl₃) δ 210.2, 170.8, 162.9, 162.4, 104.8, 98.6, 96.1, 57.9, 50.4, 29.5, 26.1, 15.1, 1×AlkC not observed. HR-MS (positive mode): calculated for [M+H]⁺ 343.1210; Found 343.1215.

### 1,1′-(((2,4,6-Trihydroxy-5-(phenylsulfonyl)-1,3-phenylene)bis(methylene))bis(2,4-dihydroxy-6-methoxy-5-methyl-3,1-phenylene))bis(butan-1-one)

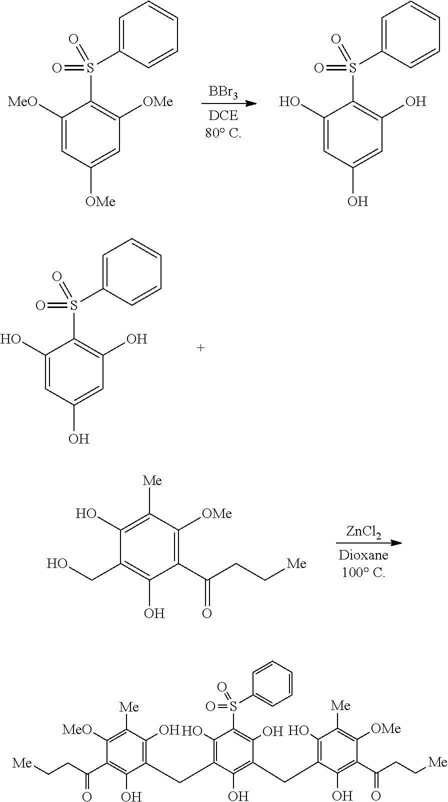

A solution of sulfone (20 mg, 0.065 mmol, 1.0 equiv.) in 1,2-dichloroethane (0.65 mL) was added with BBr₃ (0.07 mL, 0.73 mmol, 11 equiv.) and stirred at 80° C. for 1 h with a N₂ ballon. The reaction was diluted with EtOAc and slowly transferred to H₂O at 0° C. After separation, the aqueous phase was extracted with EtOAc. The combined organic phases were dried (Na₂SO₄), filtered and concentrated to afford a crude of 2-(phenylsulfonyl)benzene-1,3,5-triol which was used directly in the next step. ¹H NMR (600 MHz, MeOD) δ 7.93 (d, J=7.5 Hz, 2H), 7.61 (t, J=7.5 Hz, 1H), 7.53 (t, J=7.5 Hz, 2H), 5.83 (s, 2H). ¹³C NMR (151 MHz, MeOD) δ 166.2, 160.5, 144.1, 134.2, 129.7, 128.3, 103.2, 96.4. HR-MS (negative mode): calculated for [M−H]⁻ 265.0176; Found 265.0175.

After adding benzyl alcohol (33 mg, 0.13 mmol, 2.0 equiv.), ZnCl₂ (18 mg, 0.13 mmol, 2.0 equiv.) and dioxane (1.3 mL), the mixture was stirred at 100° C. for 2 h. The reaction was quenched with H₂O/brine and extracted with EtOAc (×2). The combined organic phases were dried (Na₂SO₄), filtered, and concentrated. Purification by flash column chromatography (0-10-30% EtOAc in cyclohexane) afforded the compound (12 mg, 25% over 2 steps). ¹H NMR (600 MHz, CDCl₃) δ 15.79 (br. s, 2H), 10.76 (app. br. s, 3H), 9.04 (br. s, 2H), 7.99 (d, J=7.5 Hz, 2H), 7.57 (t, J=7.5 Hz, 1H), 7.49 (t, J=7.5 Hz, 2H), 3.76 (s, 4H), 3.71 (s, 6H), 3.07 (t, J=7.5 Hz, 4H), 2.11 (s, 6H), 1.73 (app. h, J=7.5 Hz, 4H), 0.98 (t, J=7.5 Hz, 6H). ¹³C NMR (151 MHz, CDCl₃) δ 207.3, 161.6, 160.5, 159.6, 158.1, 154.2, 141.8, 133.5, 128.7, 128.1, 112.5, 109.0, 108.0, 107.0, 105.0, 61.8, 44.3, 18.3, 16.9, 14.1, 9.3. HR-MS (negative mode): calculated for [M−H]⁻ 737.2273; Found 737.2272.

### General Procedure for A-B Ring Synthesis Coupling (Method D)

### 1,1′-(((2,4,6-Trihydroxy-5-tosyl-1,3-phenylene)bis(methylene))bis(2,4-dihydroxy-6-methoxy-5-methyl-3,1-phenylene))bis(butan-1-one)

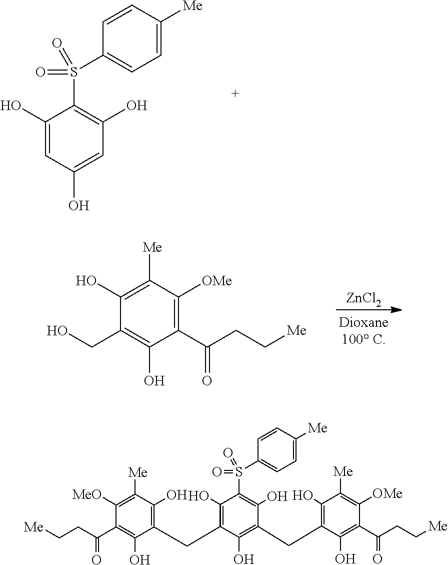 [SMILES: Cc1ccc(S(=O)(=O)c2c(O)cc(O)cc2O)cc1 ; CCCC(=O)c1c(O)c(CO)c(O)c(C)c1OC ; "ZnCl2" ; CCCC(=O)c1c(O)c(Cc2c(O)c(Cc3c(O)c(C)c(OC)c(C(=O)CCC)c3O)c(O)c(S(=O)(=O)c3ccc(C)cc3)c2O)c(O)c(C)c1OC]

A mixture of sulfone (14 mg, 0.050 mmol, 1.0 equiv.), benzyl alcohol (38 mg, 0.15 mmol, 3.0 equiv.), and ZnCl₂ (20 mg, 0.15 mmol, 3.0 equiv.) in dioxane (5.0 mL) was stirred at 100° C. for 1 h. The reaction was quenched with H₂O/brine and extracted with EtOAc (×2). The combined organic phases were dried (Na₂SO₄), filtered, and concentrated. Purification by flash column chromatography (0-60% EtOAc in n-hexane) afforded the compound (25 mg, 66%). ¹H NMR (600 MHz, CDCl₃) δ 15.79 (s, 2H), 11.16-10.37 (app. br. s, 3H), 9.07 (s, 2H), 7.87 (d, J=8.5 Hz, 2H), 7.28 (d, J=8.5 Hz, 2H), 3.76 (s, 4H), 3.72 (s, 6H), 3.08 (t, J=7.5 Hz, 4H), 2.40 (s, 3H), 2.11 (s, 6H), 1.73 (app. h, J=7.5 Hz, 4H), 0.98 (t, J=7.5 Hz, 6H). ¹³C NMR (151 MHz, CDCl₃) δ 207.2, 161.6, 160.5, 159.6, 157.9, 154.0, 144.5, 138.8, 129.3, 128.2, 112.5, 109.0, 108.0, 106.9, 105.3, 61.8, 44.3, 21.8, 18.3, 16.9, 14.0, 9.3. HR-MS (negative mode): calculated for [M−H]⁻ 751.2429; Found 751.2433.

### 1-(2,4-Dihydroxy-6-methoxy-5-methyl-3-(2,4,6-trihydroxy-3-methyl-5-tosylbenzyl)phenyl)butan-1-one

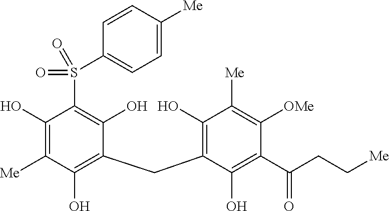 [SMILES: CCCC(=O)c1c(O)c(Cc2c(O)c(C)c(O)c(S(=O)(=O)c3ccc(C)cc3)c2O)c(O)c(C)c1OC]

Following method D using sulfone (29 mg, 0.10 mmol, 1.0 equiv.), benzyl alcohol (25 mg, 0.10 mmol, 1.0 equiv.), ZnCl₂ (14 mg, 0.10 mmol, 1.0 equiv.) and dioxane (10 mL). Purification by flash column chromatography (0-50% EtOAc in n-hexane) afforded the compound (18 mg, 34%). ¹H NMR (600 MHz, CDCl₃) δ 15.54 (s, 1H), 10.18 (s, 1H), 9.42 (s, 1H), 8.73 (s, 1H), 8.63 (s, 1H), 7.78 (d, J=8.5 Hz, 2H), 7.32 (d, J=8.5 Hz, 2H), 3.80 (s, 2H), 3.71 (s, 3H), 3.07 (t, J=7.5 Hz, 2H), 2.41 (s, 3H), 2.10 (s, 3H), 1.99 (s, 3H), 1.72 (app. h, J=7.5 Hz, 2H), 0.98 (t, J=7.5 Hz, 3H). ¹³C NMR (151 MHz, CDCl₃) δ 207.1, 161.7, 161.4, 160.5, 159.6, 153.6, 149.9, 145.4, 139.0, 130.3, 125.9, 112.1, 108.8, 108.1, 107.9, 106.3, 101.1, 61.7, 44.5, 21.8, 18.3, 16.5, 14.1, 9.3, 8.2. HR-MS (negative mode): calculated for [M−H]⁻ 529.1537; Found 529.1541.

### 1-(2,4-Dihydroxy-6-methoxy-5-methyl-3-(2,4,6-trihydroxy-3-((4-methoxyphenyl)sulfonyl)-5-methylbenzyl)phenyl)butan-1-one

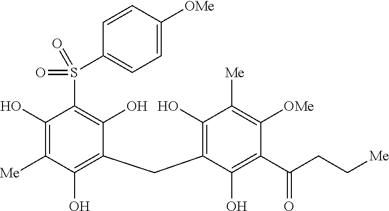 [SMILES: CCCC(=O)c1c(O)c(Cc2c(O)c(C)c(O)c(S(=O)(=O)c3ccc(OC)cc3)c2O)c(O)c(C)c1OC]

Following method D using sulfone (16 mg, 0.050 mmol, 1.0 equiv.), benzyl alcohol (31 mg, 0.12 mmol, 2.4 equiv.), ZnCl₂ (34 mg, 0.25 mmol, 5.0 equiv.), and dioxane (10 mL). Purification by flash column chromatography (0-30-40% EtOAc in n-hexane) afforded the compound (14 mg, 51%). ¹H NMR (600 MHz, CDCl₃) δ 15.53 (s, 1H), 10.19 (br. s, 1H), 9.40 (s, 1H), 8.75 (br. s, 1H), 8.63 (s, 1H), 7.84 (d, J=9.0 Hz, 2H), 6.98 (d, J=9.0 Hz, 2H), 3.85 (s, 3H), 3.79 (s, 2H), 3.71 (s, 3H), 3.07 (t, J=7.5 Hz, 2H), 2.10 (s, 3H), 1.99 (s, 3H), 1.72 (app. h, J=7.5 Hz, 2H), 0.98 (t, J=7.5 Hz, 3H). ¹³C NMR (151 MHz, CDCl₃) δ 207.1, 164.2, 161.43, 161.39, 160.5, 159.6, 153.4, 149.8, 133.5, 128.2, 114.9, 112.1, 108.8, 108.1, 107.8, 106.3, 101.7, 61.7, 55.9, 44.5, 18.3, 16.5, 14.1, 9.3, 8.2. HR-MS (negative mode): calculated for [M−H]⁻ 545.1486; Found 545.1489.

### 1,1′-(((2,4,6-Trihydroxy-5-(methylsulfonyl)-1,3-phenylene)bis(methylene))bis(2,4-dihydroxy-6-methoxy-5-methyl-3,1-phenylene))bis(butan-1-one)

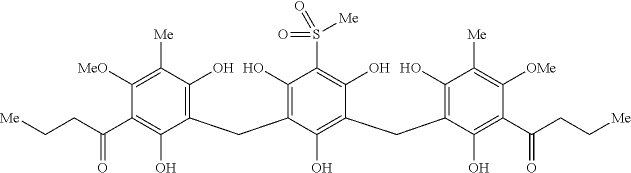 [SMILES: CCCC(=O)c1c(O)c(Cc2c(O)c(Cc3c(O)c(C)c(OC)c(C(=O)CCC)c3O)c(O)c(S(C)(=O)=O)c2O)c(O)c(C)c1OC]

Following method D using sulfone (19 mg, 0.093 mmol, 1.0 equiv.), benzyl alcohol (52 mg, 0.20 mmol, 2.2 equiv.), ZnCl₂ (28 mg, 0.21 mmol, 2.1 equiv.), and dioxane (2.0 mL). Purification by flash column chromatography (0-25-50% EtOAc in cyclohexane) afforded the compound (50 mg, 80%). ¹H NMR (600 MHz, CDCl₃) δ 15.94 (br. s, 2H), 10.81 (app. br. s, 3H), 9.01 (s, 2H), 3.81 (s, 4H), 3.72 (s, 6H), 3.37 (s, 3H), 3.09 (t, J=7.5 Hz, 4H), 2.11 (s, 6H), 1.74 (app. h, J=7.5 Hz, 4H), 0.99 (t, J=7.5 Hz, 6H). ¹³C NMR (151 MHz, CDCl₃) δ 207.3, 161.6, 160.6, 159.6, 157.8, 154.0, 112.5, 109.0, 108.0, 107.1, 104.7, 61.7, 45.0, 44.3, 18.3, 16.9, 14.1, 9.3. HR-MS (positive mode): calculated for [M+H]⁺ 677.2263; Found 677.2264.

### 1,1′-(((2,4,6-Trihydroxy-5-(propylsulfonyl)-1,3-phenylene)bis(methylene))bis(2,4-dihydroxy-6-methoxy-5-methyl-3,1-phenylene))bis(butan-1-one)

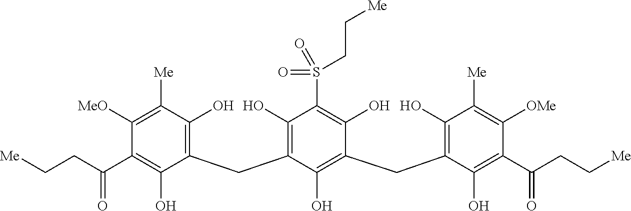 [SMILES: CCCC(=O)c1c(O)c(Cc2c(O)c(Cc3c(O)c(C)c(OC)c(C(=O)CCC)c3O)c(O)c(S(=O)(=O)CCC)c2O)c(O)c(C)c1OC]

Following method D using sulfone (31 mg, 0.13 mmol, 1.0 equiv.), benzyl alcohol (81 mg, 0.32 mmol, 2.4 equiv.), ZnCl₂ (44 mg, 0.32 mmol, 2.4 equiv.), and dioxane (2.7 mL). Purification by flash column chromatography (0-20-50% EtOAc in n-hexane) afforded the compound (67 mg, 71%). ¹H NMR (600 MHz, CDCl₃) δ 15.91 (br. s, 2H), 10.79 (app. br. s, 3H), 9.03 (s, 2H), 3.80 (s, 4H), 3.72 (s, 6H), 3.49-3.44 (m, 2H), 3.09 (t, J=7.5 Hz, 4H), 2.11 (s, 6H), 1.83-1.76 (m, 2H), 1.76-1.70 (m, 4H), 1.02 (t, J=7.5 Hz, 3H), 0.99 (t, J=7.5 Hz, 6H). ¹³C NMR (151 MHz, CDCl₃) δ 207.3, 161.6, 160.5, 159.6, 157.8, 154.4, 112.5, 109.0, 108.0, 107.0, 102.9, 61.7, 57.8, 44.3, 18.3, 16.9, 16.3, 14.0, 13.0, 9.3. HR-MS (positive mode): calculated for [M+H]⁺ 705.2576; Found 705.2573.

### 1,1′-(((2,4,6-Trihydroxy-5-(isopropylsulfonyl)-1,3-phenylene)bis(methylene))bis(2,4-dihydroxy-6-methoxy-5-methyl-3,1-phenylene))bis(butan-1-one)

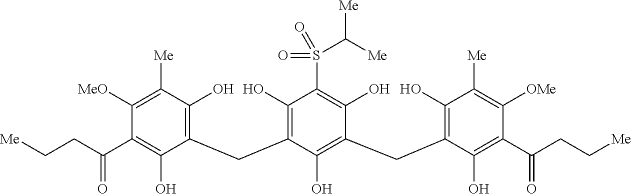 [SMILES: CCCC(=O)c1c(O)c(Cc2c(O)c(Cc3c(O)c(C)c(OC)c(C(=O)CCC)c3O)c(O)c(S(=O)(=O)C(C)C)c2O)c(O)c(C)c1OC]

Following method D using sulfone (23 mg, 0.10 mmol, 1.0 equiv.), benzyl alcohol (56 mg, 0.22 mmol, 2.2 equiv.), ZnCl₂ (30 mg, 0.22 mmol, 2.2 equiv.), and dioxane (2.0 mL). Purification by flash column chromatography (0-50% EtOAc in n-hexane) afforded the compound (39 mg, 56%). ¹H NMR (600 MHz, CDCl₃) δ 15.89 (br. s, 2H), 10.78 (app. br. s, 3H), 9.05 (s, 2H), 3.93 (hept, J=7.0 Hz, 1H), 3.80 (s, 4H), 3.73 (s, 6H), 3.09 (t, J=7.5 Hz, 4H), 2.11 (s, 6H), 1.74 (app. h, J=7.5 Hz, 4H), 1.34 (d, J=7.0 Hz, 6H), 0.99 (t, J=7.5 Hz, 6H). ¹³C NMR (151 MHz, CDCl₃) δ 207.3, 161.7, 160.6, 159.6, 157.8, 154.8, 112.5, 109.0, 108.0, 107.0, 101.1, 61.7, 54.8, 44.4, 18.3, 17.0, 15.0, 14.1, 9.3. HR-MS (positive mode): calculated for [M+H]⁺ 705.2576; Found 705.2572.

### 1,1′-(((5-(sec-Butylsulfonyl)-2,4,6-trihydroxy-1,3-phenylene)bis(methylene))bis(2,4-dihydroxy-6-methoxy-5-methyl-3,1-phenylene))bis(butan-1-one)

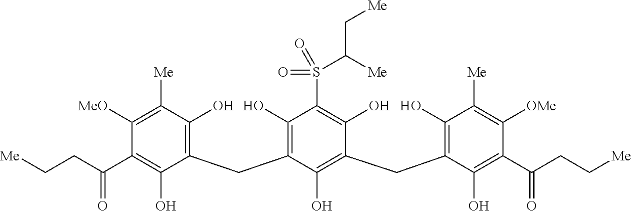 [SMILES: CCCC(=O)c1c(O)c(Cc2c(O)c(Cc3c(O)c(C)c(OC)c(C(=O)CCC)c3O)c(O)c(S(=O)(=O)C(C)CC)c2O)c(O)c(C)c1OC]

Following method D using sulfone (36 mg, 0.15 mmol, 1.0 equiv.), benzyl alcohol (89 mg, 0.35 mmol, 2.4 equiv.), ZnCl₂ (48 mg, 0.35 mmol, 2.4 equiv.), and dioxane (3.0 mL). Purification by flash column chromatography (0-10-20-30% EtOAc in n-hexane) afforded the compound (24 mg, 23%). ¹H NMR (600 MHz, CDCl₃) δ 15.89 (s, 2H), 10.77 (app. br. s, 3H), 9.06 (s, 2H), 3.80 (s, 4H), 3.73 (s, 6H), 3.71-3.65 (m, 1H), 3.09 (t, J=7.5 Hz, 4H), 2.11 (s, 6H), 2.02-1.92 (m, 1H), 1.74 (app. h, J=7.5 Hz, 4H), 1.65-1.55 (m, 1H), 1.31 (d, J=7.0 Hz, 3H), 1.03-0.96 (m, 9H). ¹³C NMR (151 MHz, CDCl₃) δ 207.3, 161.7, 160.5, 159.6, 157.8, 154.8, 112.5, 109.0, 108.0, 107.0, 101.5, 61.7, 60.7, 44.4, 21.9, 18.3, 17.0, 14.1, 12.0, 11.2, 9.3. HR-MS (negative mode): calculated for [M−H]⁻ 717.2586; Found 717.2590.

### 1,1′-(((5-(Cyclohexylsulfonyl)-2,4,6-trihydroxy-1,3-phenylene)bis(methylene))bis(2,4-dihydroxy-6-methoxy-5-methyl-3,1-phenylene))bis(butan-1-one)

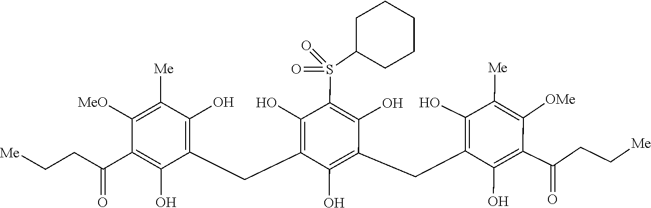 [SMILES: CCCC(=O)c1c(O)c(Cc2c(O)c(Cc3c(O)c(C)c(OC)c(C(=O)CCC)c3O)c(O)c(S(=O)(=O)C3CCCCC3)c2O)c(O)c(C)c1OC]

Following method D using sulfone (38 mg, 0.14 mmol, 1.0 equiv.), benzyl alcohol (86 mg, 0.34 mmol, 2.4 equiv.), ZnCl₂ (46 mg, 0.34 mmol, 2.4 equiv.), and dioxane (2.8 mL). Purification by flash column chromatography (0-15% EtOAc in n-hexane) afforded the compound (51 mg, 49%). ¹H NMR (600 MHz, CDCl₃) δ 15.88 (s, 2H), 10.76 (app. br. s, 3H), 9.06 (s, 2H), 3.80 (s, 4H), 3.72 (s, 6H), 3.63 (tt, J=12.5, 3.5 Hz, 1H), 3.08 (t, J=7.5 Hz, 4H), 2.11 (s, 6H), 2.05-1.98 (m, 2H), 1.90-1.85 (m, 2H), 1.80-1.65 (m, 6H), 1.60-1.52 (m, 2H), 1.34-1.17 (m, 2H), 0.99 (t, J=7.5 Hz, 6H). ¹³C NMR (151 MHz, CDCl₃) δ 207.3, 161.7, 160.5, 159.6, 157.8, 154.8, 112.5, 109.0, 108.0, 106.9, 101.2, 62.6, 61.8, 44.4, 25.3, 25.1, 24.7, 18.3, 17.0, 14.1, 9.3. HR-MS (negative mode): calculated for [M−H]⁻ 743.2742; Found 743.2748.

### 1-(2,4-Dihydroxy-6-methoxy-5-methyl-3-(2,4,6-trihydroxy-3-isobutyryl-5-tosylbenzyl)phenyl) butan-1-one

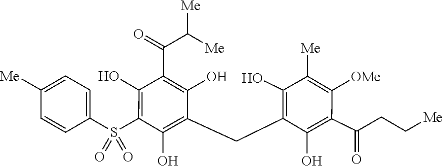 [SMILES: CCCC(=O)c1c(O)c(Cc2c(O)c(C(=O)C(C)C)c(O)c(S(=O)(=O)c3ccc(C)cc3)c2O)c(O)c(C)c1OC]

Following method D using sulfone (15 mg, 0.043 mmol, 1.0 equiv.), benzyl alcohol (17 mg, 0.067 mmol, 1.6 equiv.), ZnCl₂ (9 mg, 0.066 mmol, 1.5 equiv.), and dioxane (1.9 mL). Purification by flash column chromatography (0-15-30% EtOAc in cyclohexane) afforded the compound (23 mg, 91%). ¹H NMR (600 MHz, CDCl₃) δ 15.76 (br. s, 1H), 12.72 (br. s, 1H), 10.73 (br. s, 1H), 8.98 (br. s, 1H), 7.88 (d, J=8.5 Hz, 2H), 7.31 (d, J=8.5 Hz, 2H), 3.94 (hept, J=7.0 Hz, 1H), 3.72 (s, 3H), 3.71 (s, 2H), 3.07 (t, J=7.5 Hz, 2H), 2.42 (s, 3H), 2.10 (s, 3H), 1.73 (app. h, J=7.5 Hz, 2H), 1.18 (d, J=6.5 Hz, 6H), 0.98 (t, J=7.5 Hz, 3H). ¹³C NMR (151 MHz, CDCl₃) δ 212.4, 207.2, 161.6, 160.6, 159.5, 144.8, 138.8, 129.4, 128.2, 112.5, 108.6, 108.1, 106.1, 104.1, 104.0, 61.7, 44.3, 39.8, 21.8, 19.3, 18.3, 16.0, 14.0, 9.3, 3×ArC not observed. HR-MS (positive mode): calculated for [M+H]⁺ 587.1946; Found 587.1950.

### 1-(2,4-Dihydroxy-6-methoxy-5-methyl-3-(2,4,6-trihydroxy-3-isobutyryl-5-(phenylsulfonyl)benzyl)phenyl)butan-1-one

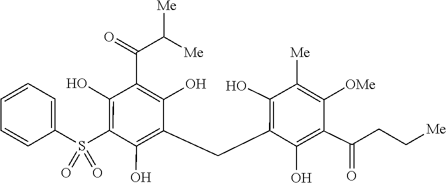 [SMILES: CCCC(=O)c1c(O)c(Cc2c(O)c(C(=O)C(C)C)c(O)c(S(=O)(=O)c3ccccc3)c2O)c(O)c(C)c1OC]

Following method D using sulfone (50 mg, 0.15 mmol, 1.0 equiv.), benzyl alcohol (43 mg, 0.17 mmol, 1.1 equiv.), ZnCl₂ (23 mg, 0.17 mmol, 1.1 equiv.), and dioxane (3.0 mL). Purification by flash column chromatography (0-10-20-40% EtOAc in n-hexane) afforded the compound (62 mg, 73%). ¹H NMR (600 MHz, CDCl₃) δ 15.77 (br. s, 1H), 12.35 (br. s, 1H), 10.67 (br. s, 1H), 8.97 (br. s, 1H), 8.00 (d, J=1.5 Hz, 2H), 7.61 (t, J=7.5 Hz, 1H), 7.52 (t, J=7.5 Hz, 2H), 3.94 (hept, J=6.5 Hz, 1H), 3.71 (app. s, 5H), 3.07 (t, J=7.5 Hz, 2H), 2.10 (s, 3H), 1.73 (app. h, J=7.5 Hz, 2H), 1.19 (d, J=6.5 Hz, 6H), 0.98 (t, J=7.5 Hz, 3H). ¹³C NMR (151 MHz, CDCl₃) δ 212.3, 207.2, 166.7, 162.9, 161.5, 160.6, 159.5, 141.6, 133.7, 128.8, 128.0, 112.5, 108.6, 108.0, 106.2, 104.0, 103.7, 61.7, 44.3, 39.8, 19.2, 18.2, 16.0, 14.0, 9.2, 1×ArC not observed. HR-MS (negative mode): calculated for [M−H]⁻ 571.1643; Found 571.1644.

### 1-(3-(3-(Cyclohexylsulfonyl)-2,4,6-trihydroxy-5-isobutyrylbenzyl)-2,4-dihydroxy-6-methoxy-5-methylphenyl)butan-1-one

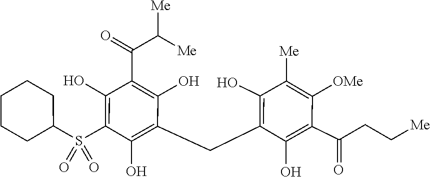 [SMILES: CCCC(=O)c1c(O)c(Cc2c(O)c(C(=O)C(C)C)c(O)c(S(=O)(=O)C3CCCCC3)c2O)c(O)c(C)c1OC]

Following method D using sulfone (31 mg, 0.091 mmol, 1.0 equiv.), benzyl alcohol (23 mg, 0.091 mmol, 1.0 equiv.), ZnCl₂ (12 mg, 0.09 mmol, 1.0 equiv.), and dioxane (1.8 mL). Purification by flash column chromatography (0-20-30% EtOAc in n-hexane) afforded the compound (41 mg, 78%). ¹H NMR (600 MHz, CDCl₃) δ 15.89 (br. s, 1H), 12.74 (br. s, 1H), 11.09 (br. s, 1H), 8.99 (br. s, 1H), 3.91 (hept, J=6.5 Hz, 1H), 3.79 (s, 2H), 3.73 (s, 3H), 3.63 (tt, J=12.0, 3.5 Hz, 1H), 3.08 (t, J=7.5 Hz, 2H), 2.12 (s, 3H), 2.04 (d, J=12.5 Hz, 2H), 1.90 (dt, J=13.5, 3.5 Hz, 2H), 1.78-1.69 (m, 3H), 1.63-1.55 (m, 2H), 1.36-1.27 (m, 2H), 1.23-1.19 (m, 1H), 1.17 (d, J=6.5 Hz, 6H), 0.99 (t, J=7.5 Hz, 3H). ¹³C NMR (151 MHz, CDCl₃) δ 212.5, 207.3, 161.6, 161.0, 160.7, 159.6, 112.5, 108.6, 108.1, 106.2, 104.0, 99.8, 62.8, 61.8, 44.4, 39.8, 25.3, 25.1, 24.7, 19.3, 18.3, 16.2, 14.1, 9.3, 2×ArC not observed. HR-MS (positive mode): calculated for [M+H]+ 579.2259; Found 579.2258.

### 1-(2,4-Dihydroxy-6-methoxy-5-methyl-3-(2,4,6-trihydroxy-3-isobutyryl-5-(isopropylsulfonyl)benzyl)phenyl)butan-1-one

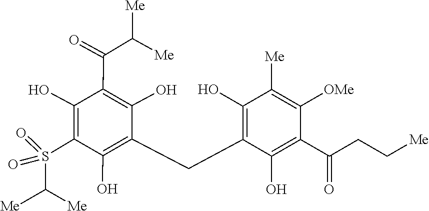 [SMILES: CCCC(=O)c1c(O)c(Cc2c(O)c(C(=O)C(C)C)c(O)c(S(=O)(=O)C(C)C)c2O)c(O)c(C)c1OC]

Following method D using sulfone (23 mg, 0.076 mmol, 1.0 equiv.), benzyl alcohol (19 mg, 0.076 mmol, 1.0 equiv.), ZnCl₂ (10 mg, 0.076 mmol, 1.0 equiv.), and dioxane (1.5 mL). Purification by flash column chromatography (0-40% EtOAc in n-hexane) afforded the compound (23 mg, 56%). ¹H NMR (600 MHz, CDCl₃) δ 15.90 (br. s, 1H), 12.50 (br. s, 1H), 11.06 (br. s, 1H), 8.96 (br. s, 1H), 3.96-3.87 (m, 2H), 3.78 (s, 2H), 3.73 (s, 3H), 3.08 (t, J=7.5 Hz, 2H), 2.11 (s, 3H), 1.73 (app. h, J=7.5 Hz, 2H), 1.36 (d, J=7.0 Hz, 6H), 1.17 (d, J=6.5 Hz, 6H), 0.99 (t, J=7.5 Hz, 3H). ¹³C NMR (151 MHz, CDCl₃) δ 212.5, 207.3, 166.6, 163.7, 161.6, 161.0, 160.7, 159.6, 112.5, 108.6, 108.1, 106.3, 104.0, 99.8, 61.7, 55.1, 44.4, 39.8, 19.3, 18.3, 16.1, 15.0, 14.1, 9.3. HR-MS (negative mode): calculated for [M−H]⁻ 537.1799; Found 537.1804.

### 1-(3-(3-(Cyclohexanecarbonyl)-2,4,6-trihydroxy-5-tosylbenzyl)-2,4-dihydroxy-6-methoxy-5-methylphenyl)butan-1-one

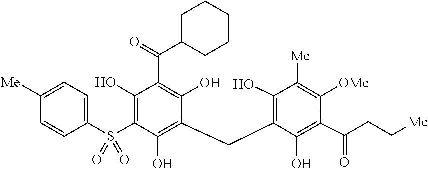 [SMILES: CCCC(=O)c1c(O)c(Cc2c(O)c(C(=O)C3CCCCC3)c(O)c(S(=O)(=O)c3ccc(C)cc3)c2O)c(O)c(C)c1OC]

Following method D using sulfone (39 mg, 0.1 mmol, 1.0 equiv.), benzyl alcohol (31 mg, 0.12 mmol, 1.2 equiv.), ZnCl₂ (16 mg, 0.12 mmol, 1.2 equiv.), and dioxane (2.0 mL). Purification by flash column chromatography (0-15-25% EtOAc in cyclohexane) afforded the compound (49 mg, 78%). ¹H NMR (600 MHz, CDCl₃) δ 15.74 (br. s, 1H), 12.43 (br. s, 1H), 10.63 (br. s, 1H), 9.02 (br. s, 1H), 7.88 (d, J=8.0 Hz, 2H), 7.31 (d, J=8.0 Hz, 2H), 3.71 (s, 3H), 3.69 (s, 2H), 3.64 (tt, J=10.5, 3.0 Hz, 1H), 3.07 (t, J=7.5 Hz, 2H), 2.42 (s, 3H), 2.10 (s, 3H), 1.91 (d, J=11.0 Hz, 2H), 1.82 (d, J=13.0 Hz, 2H), 1.76-1.69 (m, 3H), 1.45-1.31 (m, 4H), 1.29-1.20 (m, 1H), 0.98 (t, J=7.5 Hz, 3H). ¹³C NMR (151 MHz, CDCl₃) δ 211.4, 207.2, 161.6, 160.6, 159.5, 144.7, 138.8, 129.4, 128.1, 112.5, 108.6, 108.0, 106.1, 104.1, 61.7, 50.1, 44.3, 29.5, 26.13, 26.10, 21.8, 18.3, 16.0, 14.0, 9.3, 4×ArC not observed. HR-MS (positive mode): calculated for [M+H]⁺ 627.2259; Found 627.2263.

### 1-(3-(3-(Cyclohexanecarbonyl)-5-(cyclohexylsulfonyl)-2,4,6-trihydroxybenzyl)-2,4-dihydroxy-6-methoxy-5-methylphenyl)butan-1-one

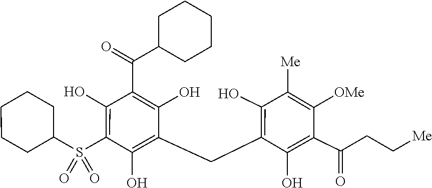 [SMILES: CCCC(=O)c1c(O)c(Cc2c(O)c(C(=O)C3CCCCC3)c(O)c(S(=O)(=O)C3CCCCC3)c2O)c(O)c(C)c1OC]

Following method D using sulfone (45 mg, 0.12 mmol, 1.0 equiv.), benzyl alcohol (36 mg, 0.14 mmol, 1.2 equiv.), ZnCl₂ (19 mg, 0.14 mmol, 1.2 equiv.), and dioxane (2.4 mL). Purification by flash column chromatography (0-20-30% EtOAc in cyclohexane) afforded the compound (20 mg, 27%). ¹H NMR (600 MHz, CDCl₃) δ 15.88 (br. s, 1H), 12.59 (br. s, 1H), 11.01 (br. s, 1H), 9.04 (br. s, 1H), 3.78 (s, 2H), 3.73 (s, 3H), 3.66-3.57 (m, 2H), 3.08 (t, J=7.5 Hz, 2H), 2.11 (s, 3H), 2.04 (d, J=12.0 Hz, 2H), 1.94-1.87 (m, 4H), 1.81 (dt, J=13.0, 3.5 Hz, 2H), 1.77-1.69 (m, 4H), 1.64-1.54 (m, 2H), 1.45-1.17 (m, 8H), 0.99 (t, J=7.5 Hz, 3H). ¹³C NMR (151 MHz, CDCl₃) δ 211.5, 207.3, 161.6, 161.0, 160.6, 159.6, 112.5, 108.7, 108.1, 106.2, 104.2, 99.8, 62.9, 61.7, 50.1, 44.4, 29.5, 26.13, 26.09, 25.3, 25.1, 24.7, 18.3, 16.2, 14.1, 9.3, 2×ArC not observed. HR-MS (negative mode): calculated for [M−H]⁻ 617.2425; Found 617.2431.

### 1-(3-(3-(Cyclohexanecarbonyl)-2,4,6-trihydroxy-5-(isopropylsulfonyl)benzyl)-2,4-dihydroxy-6-methoxy-5-methylphenyl)butan-1-one

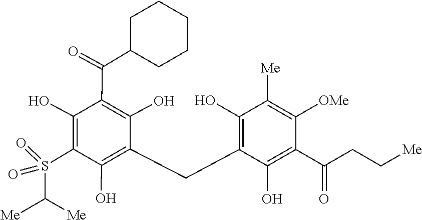 [SMILES: CCCC(=O)c1c(O)c(Cc2c(O)c(C(=O)C3CCCCC3)c(O)c(S(=O)(=O)C(C)C)c2O)c(O)c(C)c1OC]

Following method D using sulfone (20 mg, 0.058 mmol, 1.0 equiv.), benzyl alcohol (16 mg, 0.064 mmol, 1.1 equiv.), ZnCl₂ (9 mg, 0.07 mmol, 1.1 equiv.), and dioxane (1.2 mL). Purification by flash column chromatography (0-40% EtOAc in n-hexane) afforded the compound (20 mg, 59%). ¹H NMR (600 MHz, CDCl₃) δ 16.68 (br. s, 1H), 15.89 (br. s, 1H), 12.38 (br. s, 1H), 10.98 (br. s, 1H), 9.05 (br. s, 1H), 3.92 (hept, J=7.0 Hz, 1H), 3.78 (s, 2H), 3.73 (s, 3H), 3.61 (tt, J=11.0, 3.0 Hz, 1H), 3.08 (t, J=7.5 Hz, 2H), 2.11 (s, 3H), 1.90 (d, J=12.0 Hz, 2H), 1.81 (dt, J=13.0, 3.0 Hz, 2H), 1.77-1.69 (m, 3H), 1.45-1.31 (m, 10H), 1.27-1.18 (m, 1H), 0.99 (t, J=7.5 Hz, 3H). ¹³C NMR (151 MHz, CDCl₃) δ 211.5, 207.3, 166.9, 163.7, 161.6, 161.0, 160.6, 159.6, 112.5, 108.7, 108.1, 106.3, 104.1, 99.8, 61.7, 55.1, 50.1, 44.4, 29.5, 26.13, 26.09, 18.3, 16.1, 15.0, 14.1, 9.3. HR-MS (negative mode): calculated for [M−H]⁻ 577.2112; Found 577.2116.

### Results

A series of exemplary AGB derivatives incorporating sulfone or ketone/sulfone groups were prepared and characterized as described above. Further examination on NCI-H460 large lung cancer cells using these AGB derivatives revealed that some of these derivatives exhibited cytotoxicity comparable to that of the original AGB (Table 1). These results demonstrated that the sulfone-incorporated AGB derivatives are useful in cancer treatment.

TABLE 1
IC₅₀ values of AGB, AGB derivatives, and
Sulfone/Ketone-Sulfone A ring Intermediates

| Structure | NCI-H460 (μM)* |
|---|---|
| 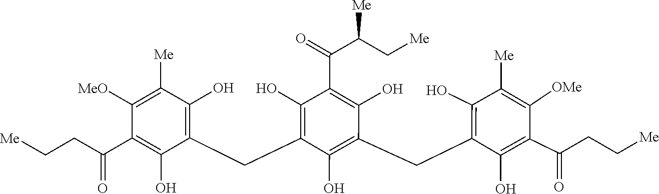 [SMILES: CCCC(=O)c1c(O)c(Cc2c(O)c(Cc3c(O)c(C)c(OC)c(C(=O)CCC)c3O)c(O)c(C(=O)[C@@H](C)CC)c2O)c(O)c(C)c1OC] | 1.30 ± 0.30 |
| Agrimol B (AGB) | |
| 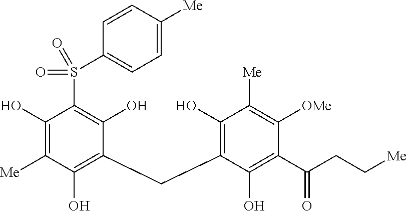 [SMILES: CCCC(=O)c1c(O)c(Cc2c(O)c(C)c(O)c(S(=O)(=O)c3ccc(C)cc3)c2O)c(O)c(C)c1OC] | 2.05 |
| 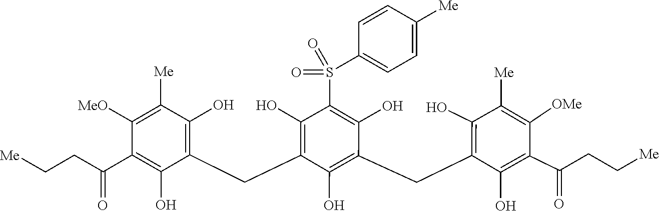 [SMILES: CCCC(=O)c1c(O)c(Cc2c(O)c(Cc3c(O)c(C)c(OC)c(C(=O)CCC)c3O)c(O)c(S(=O)(=O)c3ccc(C)cc3)c2O)c(O)c(C)c1OC] | 1.70 |
| 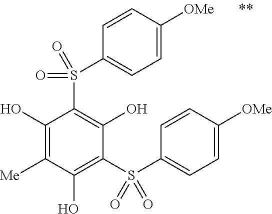 [SMILES: COc1ccc(S(=O)(=O)c2c(O)c(C)c(O)c(S(=O)(=O)c3ccc(OC)cc3)c2O)cc1] | 47.7 |
| 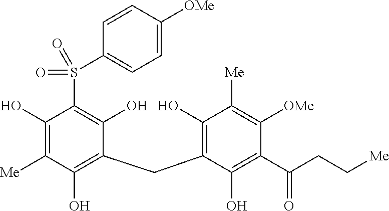 [SMILES: CCCC(=O)c1c(O)c(Cc2c(O)c(C)c(O)c(S(=O)(=O)c3ccc(OC)cc3)c2O)c(O)c(C)c1OC] | 3.44 |
| 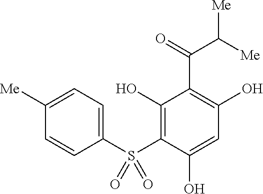 [SMILES: Cc1ccc(S(=O)(=O)c2c(O)cc(O)c(C(=O)C(C)C)c2O)cc1] | 40.9 |
| 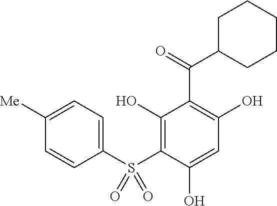 [SMILES: Cc1ccc(S(=O)(=O)c2c(O)cc(O)c(C(=O)C3CCCCC3)c2O)cc1] | 50.4 |
| 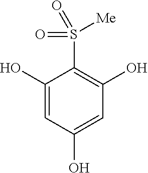 [SMILES: CS(=O)(=O)c1c(O)cc(O)cc1O] | >100 |
| 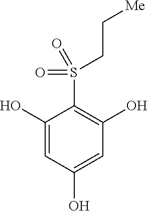 [SMILES: CCCS(=O)(=O)c1c(O)cc(O)cc1O] | >100 |
| 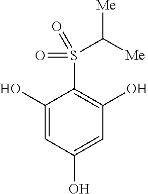 [SMILES: CC(C)S(=O)(=O)c1c(O)cc(O)cc1O] | >100 |
| 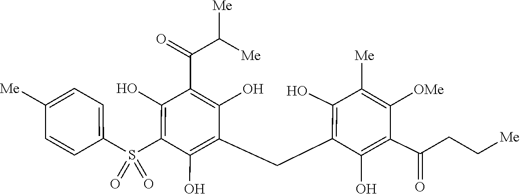 [SMILES: CCCC(=O)c1c(O)c(Cc2c(O)c(C(=O)C(C)C)c(O)c(S(=O)(=O)c3ccc(C)cc3)c2O)c(O)c(C)c1OC] | 1.70 |
| 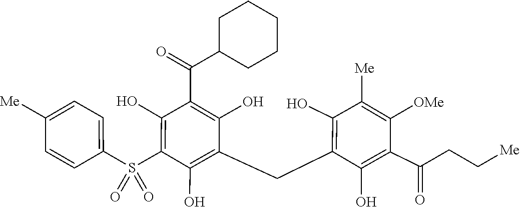 [SMILES: CCCC(=O)c1c(O)c(Cc2c(O)c(C(=O)C3CCCCC3)c(O)c(S(=O)(=O)c3ccc(C)cc3)c2O)c(O)c(C)c1OC] | 1.94 |
| 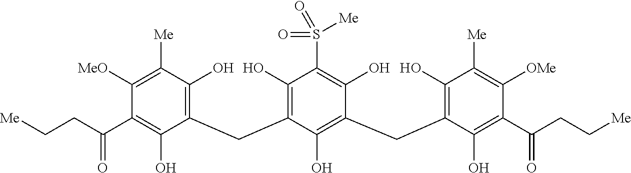 [SMILES: CCCC(=O)c1c(O)c(Cc2c(O)c(Cc3c(O)c(C)c(OC)c(C(=O)CCC)c3O)c(O)c(S(C)(=O)=O)c2O)c(O)c(C)c1OC] | 6.12 |
| 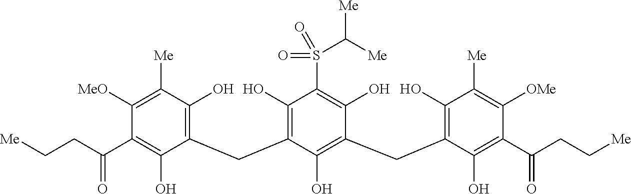 [SMILES: CCCC(=O)c1c(O)c(Cc2c(O)c(Cc3c(O)c(C)c(OC)c(C(=O)CCC)c3O)c(O)c(S(=O)(=O)C(C)C)c2O)c(O)c(C)c1OC] | 3.50 |
| 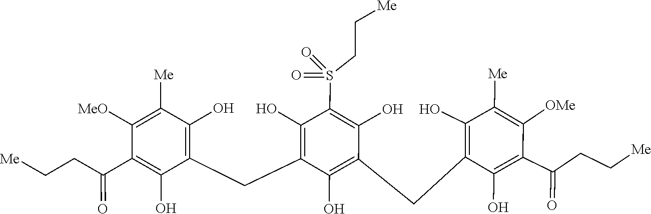 [SMILES: CCCC(=O)c1c(O)c(Cc2c(O)c(Cc3c(O)c(C)c(OC)c(C(=O)CCC)c3O)c(O)c(S(=O)(=O)CCC)c2O)c(O)c(C)c1OC] | 2.70 |
| 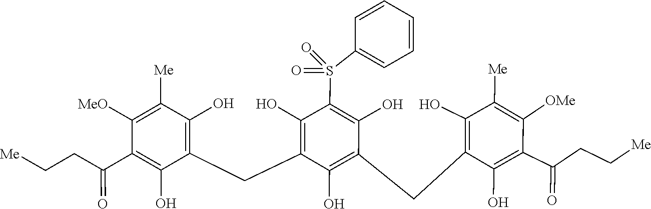 [SMILES: CCCC(=O)c1c(O)c(Cc2c(O)c(Cc3c(O)c(C)c(OC)c(C(=O)CCC)c3O)c(O)c(S(=O)(=O)c3ccccc3)c2O)c(O)c(C)c1OC] | 2.66 |
| 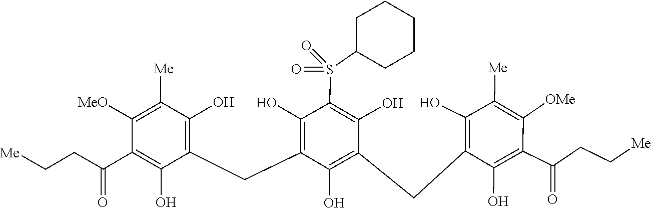 [SMILES: CCCC(=O)c1c(O)c(Cc2c(O)c(Cc3c(O)c(C)c(OC)c(C(=O)CCC)c3O)c(O)c(S(=O)(=O)C3CCCCC3)c2O)c(O)c(C)c1OC] | 2.69 |
| 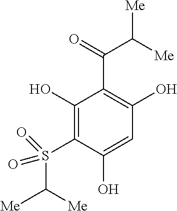 [SMILES: CC(C)C(=O)c1c(O)cc(O)c(S(=O)(=O)C(C)C)c1O] | 55.3 |
| 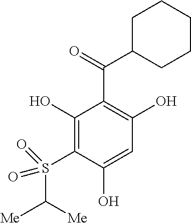 [SMILES: CC(C)S(=O)(=O)c1c(O)cc(O)c(C(=O)C2CCCCC2)c1O] | 46.6 |
| 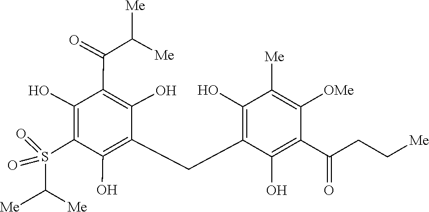 [SMILES: CCCC(=O)c1c(O)c(Cc2c(O)c(C(=O)C(C)C)c(O)c(S(=O)(=O)C(C)C)c2O)c(O)c(C)c1OC] | 1.52 |
| 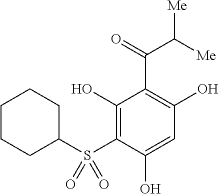 [SMILES: CC(C)C(=O)c1c(O)cc(O)c(S(=O)(=O)C2CCCCC2)c1O] | 33.2 |
| 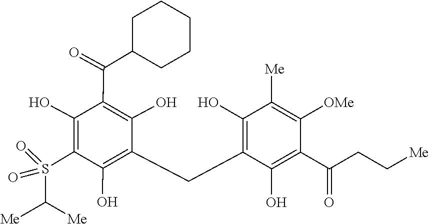 [SMILES: CCCC(=O)c1c(O)c(Cc2c(O)c(C(=O)C3CCCCC3)c(O)c(S(=O)(=O)C(C)C)c2O)c(O)c(C)c1OC] | 2.13 |
| 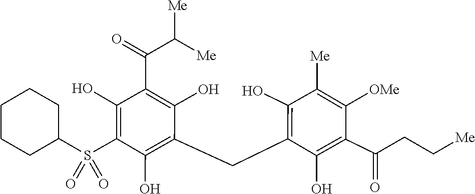 [SMILES: CCCC(=O)c1c(O)c(Cc2c(O)c(C(=O)C(C)C)c(O)c(S(=O)(=O)C3CCCCC3)c2O)c(O)c(C)c1OC] | 3.12 |
| 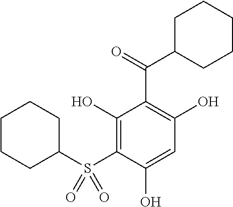 [SMILES: O=C(c1c(O)cc(O)c(S(=O)(=O)C2CCCCC2)c1O)C1CCCCC1] | 21.9 |
| 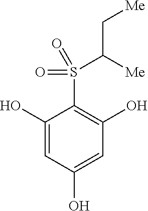 [SMILES: CCC(C)S(=O)(=O)c1c(O)cc(O)cc1O] | >100 |
| 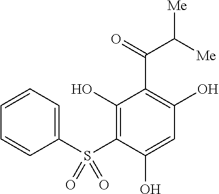 [SMILES: CC(C)C(=O)c1c(O)cc(O)c(S(=O)(=O)c2ccccc2)c1O] | 58.2 |
| 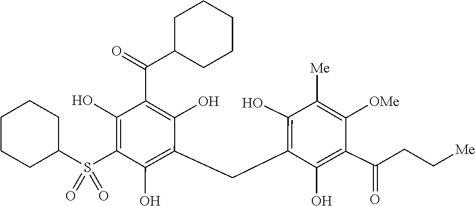 [SMILES: CCCC(=O)c1c(O)c(Cc2c(O)c(C(=O)C3CCCCC3)c(O)c(S(=O)(=O)C3CCCCC3)c2O)c(O)c(C)c1OC] | 1.60 |
| 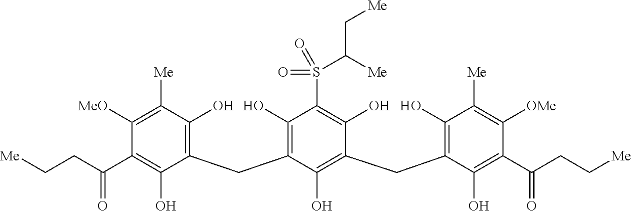 [SMILES: CCCC(=O)c1c(O)c(Cc2c(O)c(Cc3c(O)c(C)c(OC)c(C(=O)CCC)c3O)c(O)c(S(=O)(=O)C(C)CC)c2O)c(O)c(C)c1OC] | 3.15 |
| 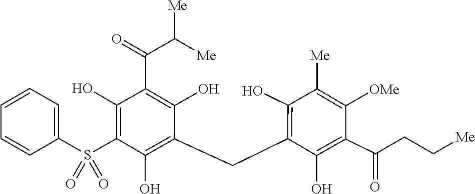 [SMILES: CCCC(=O)c1c(O)c(Cc2c(O)c(C(=O)C(C)C)c(O)c(S(=O)(=O)c3ccccc3)c2O)c(O)c(C)c1OC] | 1.61 |

*As determined by MTT method (48 h, μL).
**This is a side product formed during the preparation of A ring fragment.

### REFERENCES

Huang, J.; Shi, Y.; Tan, S.; Li, M. Preparation method of phloroglucinol derivative and intermediate. CN108264454A, 2018.

We claim:

1. A compound having the structure of: 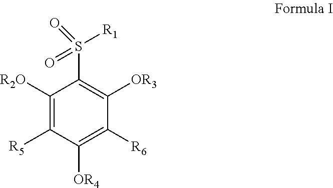
wherein:
(i) R₁ is an alkyl (e.g., an unsubstituted C₁-C₈ or C₁-C₆ alkyl, such as methyl, ethyl, n-propyl, isopropyl, n-butyl, isobutyl, tert-butyl, cyclopentyl, or cyclohexyl) or 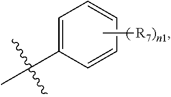
wherein
n1 is an integer from 1 to 5, and R₇ is hydrogen, an alkyl, —O-alkyl, hydroxyl, or —NR′₁R′₂, wherein R′₁ and R′₂ are independently hydrogen or an alkyl;
(ii) R₂, R₃, and R₄ are independently hydrogen or an alkyl; and 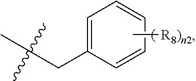
(iii) R₅ and R₆ are independently an alkyl, —C(O)—R₁₂, or, wherein
n2 is an integer from 1 to 5,
R₈ is hydrogen, hydroxyl, an alkyl, —O-alkyl, or —C(O)—R⁹,
R₉ and R₁₂ are independently hydrogen, hydroxyl, an alkyl, or —O-alkyl, and
at least one of R₅ and R₆ is

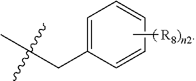

2. The compound of claim 1, wherein the 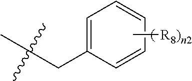
is 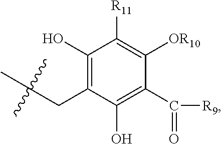
R₁₀ is hydrogen or an alkyl, and R₉ and R₁₁ are independently hydrogen, hydroxyl, or an alkyl.

3. The compound of claim 2, wherein R₉-R₁₁ are independently an alkyl (e.g., methyl, ethyl, n-propyl, isopropyl, n-butyl, isobutyl, tert-butyl, cyclopentyl, or cyclohexyl).

4. The compound of claim 1, wherein the 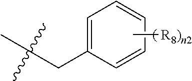
is

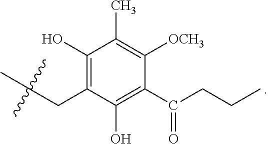 [SMILES: *Cc1c(O)c(C)c(OC)c(C(=O)CCC)c1O]

5. The compound of claim 1, wherein the 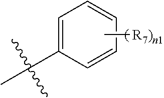
is 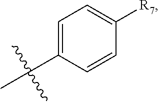
and R₇ is hydrogen, an alkyl, —O-alkyl, hydroxyl, or —NR′₁R′₂, wherein R′₁ and R′₂ are independently hydrogen or an alkyl, optionally wherein R₇ is hydrogen, an alkyl (e.g., methyl, ethyl, n-propyl, isopropyl, n-butyl, isobutyl, tert-butyl, cyclopentyl, or cyclohexyl), or —O-alkyl (e.g., methoxy or ethoxy).

6. The compound of claim 1, wherein R₂-R₄ are hydrogen.

7. The compound of claim 1, wherein R₁₂ is an alkyl (e.g., an unsubstituted C₁-C₈ or C₁-C₆ alkyl, such as methyl, ethyl, n-propyl, isopropyl, n-butyl, isobutyl, tert-butyl, cyclopentyl, or cyclohexyl).

8. The compound of claim 1, having any one of the structures of:

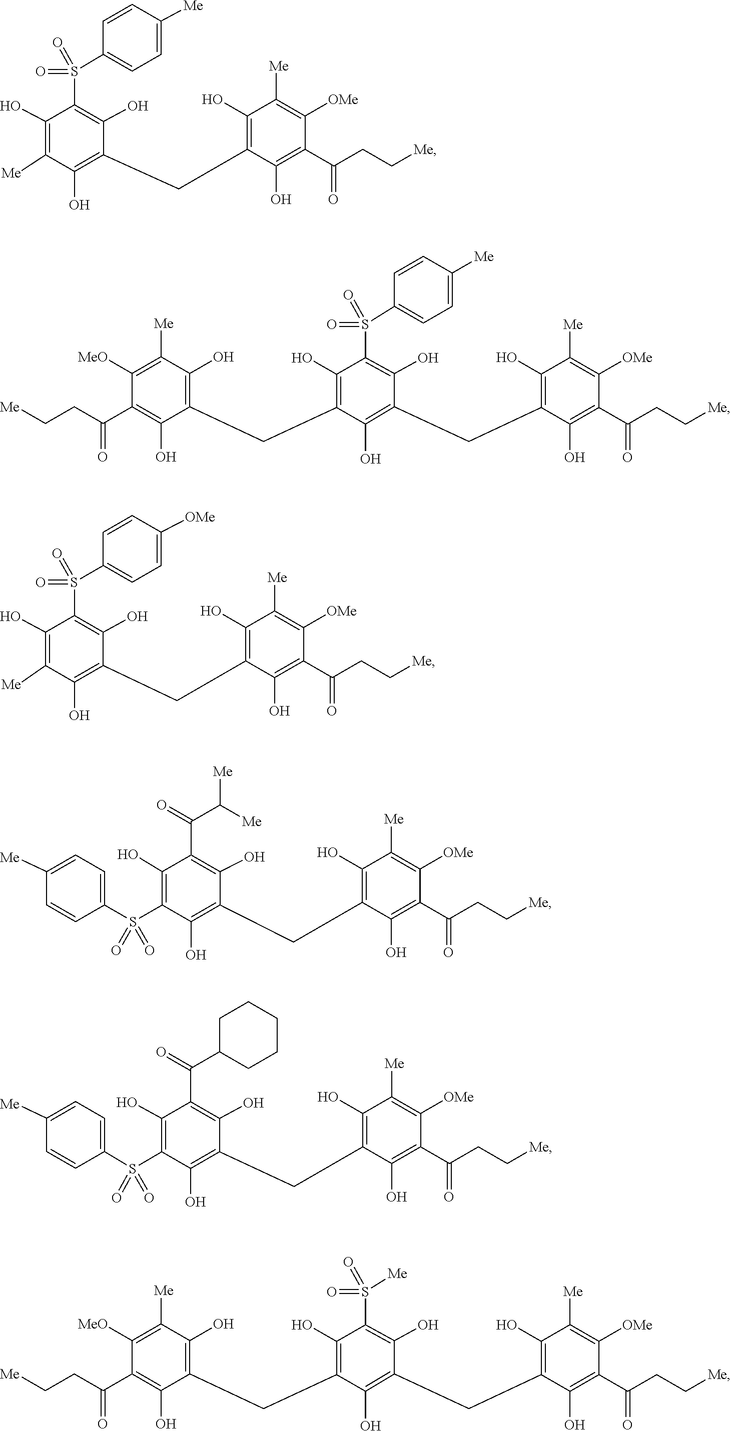

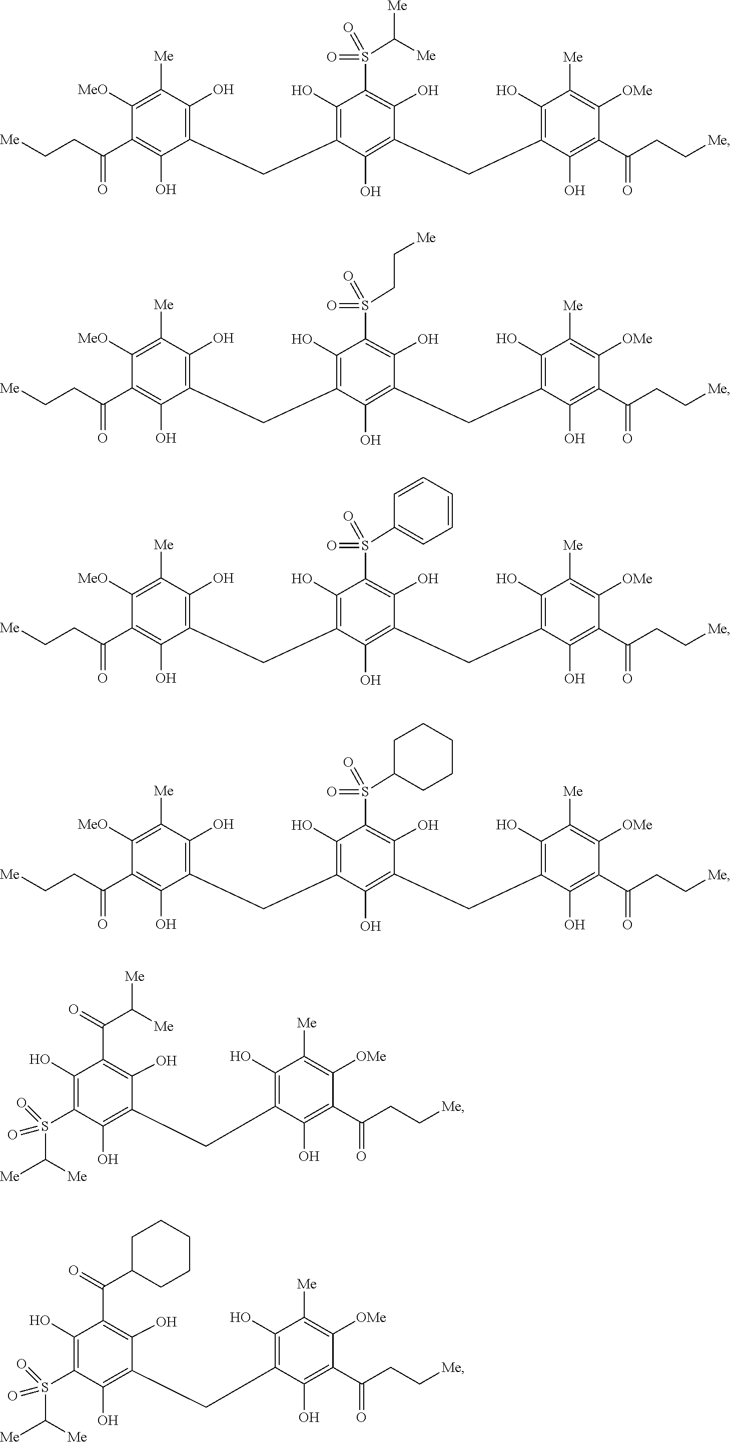

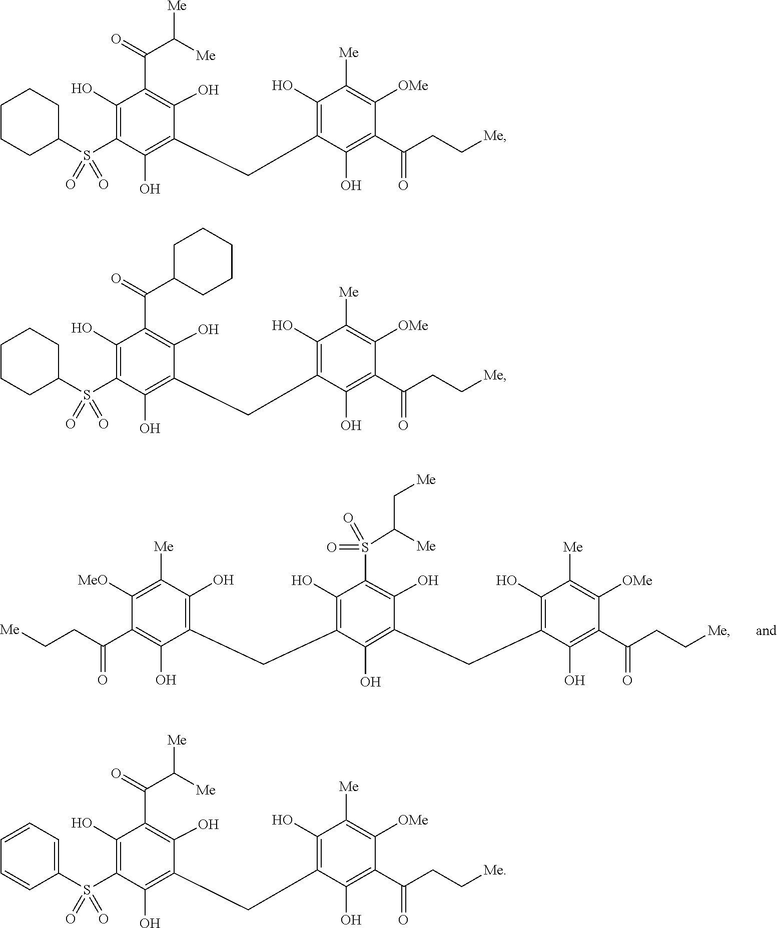 [SMILES: CCCC(=O)c1c(O)c(Cc2c(O)c(C(=O)C(C)C)c(O)c(S(=O)(=O)C3CCCCC3)c2O)c(O)c(C)c1OC ; CCCC(=O)c1c(O)c(Cc2c(O)c(C(=O)C3CCCCC3)c(O)c(S(=O)(=O)C3CCCCC3)c2O)c(O)c(C)c1OC ; CCCC(=O)c1c(O)c(Cc2c(O)c(Cc3c(O)c(C)c(OC)c(C(=O)CCC)c3O)c(O)c(S(=O)(=O)C(C)CC)c2O)c(O)c(C)c1OC ; CCCC(=O)c1c(O)c(Cc2c(O)c(C(=O)C(C)C)c(O)c(S(=O)(=O)c3ccccc3)c2O)c(O)c(C)c1OC]

9. A pharmaceutical composition comprising one or more the compound(s) of claim 1, and a pharmaceutically acceptable carrier and/or excipient.

10. The pharmaceutical composition of claim 9, wherein the one or more compounds are in an effective amount to ameliorate one or more symptoms associated with a cancer in a subject.

11. A method for synthesizing the compound of claim 1, comprising:
(1) converting a starting reagent to a sulfone intermediate or a ketone/sulfone intermediate; and
(2) converting the sulfone intermediate or ketone/sulfone intermediate to the compound,
wherein the starting reagent has the structure of: 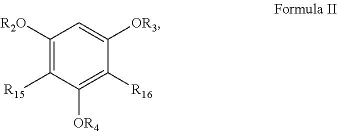
wherein the sulfone intermediate has the structure of: 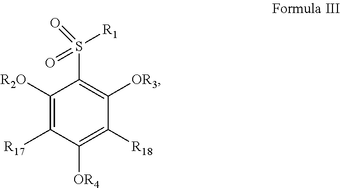
wherein the ketone/sulfone intermediate has the structure of: 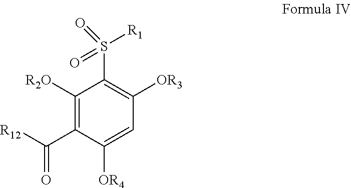
wherein:
(i) R₁ is an alkyl or 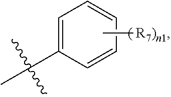
wherein n1 is an integer from 1 to 5, and R₇ is hydrogen, an alkyl, —O-alkyl, hydroxyl, or —NR′₁R′₂, wherein R′₁ and R′₂ are independently hydrogen or an alkyl;
(ii) R₂, R₃, and R₄ are independently hydrogen or an alkyl;
(iii) R₁₅ and R₁₆ are independently hydrogen or an alkyl, wherein at least one of R₁₅ and R₁₆ is hydrogen;
(iv) R₁₇ and R₁₈ are independently hydrogen, an alkyl, or 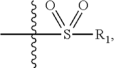
wherein at least one of R₁₇ and R₁₈ is hydrogen; and
(v) R₁₂ is hydrogen, hydroxyl, an alkyl, or —O-alkyl.

12. The method of claim 11, wherein when R₁ is 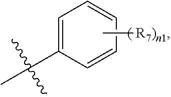
n1 is an integer from 1 to 5, R₇ is hydrogen, an alkyl, —O-alkyl, hydroxyl, or —NR′₁R′₂, and R′₁ and R′₂ are independently hydrogen or an alkyl, step (1) comprises: (a) heating a first reaction mixture at a suitable temperature for a suitable period of time to form the sulfone intermediate, wherein the first reaction mixture comprises the starting reagent and a sulfonyl halide having the structure of: 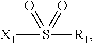
wherein X₁ is a halide (e.g., chloride, bromide, iodide, or fluoride).

13. The method of claim 11, wherein when R₁ is an alkyl, step (1) comprises:
(a) converting the starting reagent to a lithium sulfinate intermediate; and
(b) converting the lithium sulfinate intermediate to the sulfone intermediate,
wherein the lithium sulfinate intermediate has the structure of 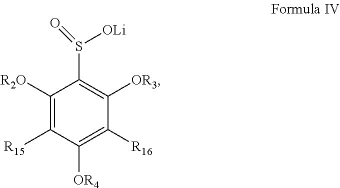
and
wherein:
(i) R₂, R₃, and R₄ are independently hydrogen or an alkyl;
(ii) R₁₅ and R₁₆ are independently hydrogen or an alkyl, wherein at least one of R₁₅ and R₁₆ is hydrogen.

14. The method of claim 13, wherein step (a) comprises mixing the starting reagent, an organolithium reagent, and a sulfur dioxide surrogate reagent to form the lithium sulfinate intermediate.

15. The method of claim 14, wherein the organolithium reagent is an alkyl-lithium (e.g., n-butyllithium), and/or the sulfur dioxide surrogate has the structure of: 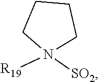
R₁₉ is an alkyl (e.g., methyl).

16. The method of claim 13, wherein step (b) comprises mixing the lithium sulfinate intermediate with an alkyl electrophile to form the sulfone intermediate, optionally wherein the alkyl electrophile is alkyl-X₂, X₂ is a halide (e.g., e.g., chloride, bromide, iodide, or fluoride).

17. The method of claim 12, wherein step (1) further comprises (c) heating a second reaction mixture at a suitable temperature for a suitable period of time to form the ketone/sulfone intermediate, wherein the second reaction mixture comprises the sulfone intermediate and a ketone reagent, and optionally wherein the ketone reagent is R₁₂—C(O)—X₃, R₁₂ is hydrogen, hydroxyl, an alkyl, or —O-alkyl, and X₃ is a halide (e.g., e.g., chloride, bromide, iodide, or fluoride).

18. The method of claim 11, wherein step (2) comprises (d) heating a third reaction mixture at a suitable temperature for a suitable period of time to form the compound, wherein the third reaction mixture comprises the sulfone intermediate or ketone/sulfone intermediate, and an aryl reagent having the structure of 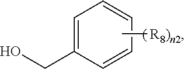
n2 is an integer from 1 to 5, R₈ is hydrogen, hydroxyl, an alkyl, —O-alkyl, or —C(O)—R₉, and R₉ is hydrogen, hydroxyl, an alkyl, or —O-alkyl.

19. A method for treating a cancer in a subject comprising:
(i) administering to the subject the pharmaceutical composition of claim 9, wherein step (i) occurs one or more times.

20. The method of claim 19, wherein the cancer is large cell lung cancer.
